SEQUENCE LISTING (24 sequences)

SEQ ID NO: 1 - protein, 153 aa, Homo sapiens
  REGION 1..153: note = human IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]

SEQ ID NO: 3 - protein, 162 aa, Homo sapiens
  REGION 1..162: note = human IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 114 aa, Homo sapiens
  REGION 1..114: note = mature human IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 267 aa, Homo sapiens
  REGION 1..267: note = human IL-15Ralpha
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 6 - protein, 61 aa, Homo sapiens
  REGION 1..61: note = human sushi domain of IL-15Ralpha
CPPPMSVEHADIWVKSYSLYSRERYICNSGFKRKAGTSSLTECVLNKATNVAHWTTPSLKC

SEQ ID NO: 7 - protein, 77 aa, Homo sapiens
  REGION 1..77: note = sushi+ fragment of IL-15Ralpha
ITCPPPMSVEHADIWVKSYSLYSRERYICNSGFKRKAGTSSLTECVLNKATNVAHWTTPSLKCIRDPALVHQRPAPP

SEQ ID NO: 8 - protein, 20 aa, synthetic construct
  REGION 1..20: note = linker
SGGSGGGGSGGGSGGGGSGG

SEQ ID NO: 9 - protein, 211 aa, synthetic construct
  REGION 1..211: note = RLI2
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 132 aa, synthetic construct
  REGION 1..132: note = IL2v
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRITFAQSIISTLT

SEQ ID NO: 11 - protein, 20 aa, synthetic construct
  REGION 1..20: note = Leader peptide of (IL-15N72D)2:IL-15Ralpha sushi-Fc:
METDTLLLWVLLLWVPGSTG

SEQ ID NO: 12 - protein, 297 aa, synthetic construct
  REGION 1..297: note = IL-15Ralpha sushi (65aa)-Fc (IgG1 CH2-CH3):
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 114 aa, synthetic construct
  REGION 1..114: note = IL-15N72D
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 14 - protein, 5 aa, synthetic construct
  REGION 1..5: note = hCl1a HCDR1
DYAMH

SEQ ID NO: 15 - protein, 17 aa, synthetic construct
  REGION 1..17: note = hCl1a HCDR2
WINTYTGKPTYAQKFQG

SEQ ID NO: 16 - protein, 10 aa, synthetic construct
  REGION 1..10: note = hCl1a HCDR3
AVFYGYTMDA

SEQ ID NO: 17 - protein, 11 aa, synthetic construct
  REGION 1..11: note = hCl1a LCDR1
RASEDIYSNLA

SEQ ID NO: 18 - protein, 7 aa, synthetic construct
  REGION 1..7: note = hCl1a LCDR2
SVKRLQD

SEQ ID NO: 19 - protein, 9 aa, synthetic construct
  REGION 1..9: note = hCl1a LCDR3
LQGSNFPLT

SEQ ID NO: 20 - protein, 449 aa, synthetic construct
  REGION 1..449: note = hCl1a HC
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 214 aa, synthetic construct
  REGION 1..214: note = hCl1a LC
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 449 aa, synthetic construct
  REGION 1..449: note = hCl1a HC LALA
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 225 aa, synthetic construct
  REGION 1..225: note = hCl1a LC-sortase tag
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSLPQTGG

SEQ ID NO: 24 - protein, 11 aa, synthetic construct
  REGION 1..11: note = Sortase Linker
GGGGSLPQTGG

(19) United States
(12) Patent Application Publication
(10) Pub. No.: US 2024/0350589 A1
(43) Pub. Date: Oct. 24, 2024

(54) IL-2/IL-15RBETAGAMMA AGONIST COMBINATION WITH ANTIBODY-DRUG CONJUGATES FOR TREATING CANCER

(71) Applicant: Cytune Pharma, Nantes (FR)

(72) Inventors: Lenka Kyrych Sadilkova, Horomerice (CZ); Ulrich Moebius, Gauting-Unterbrunn (DE); David Béchard, Saint-Etienne de Montluc (FR); Irena Adkins, Prezletice (CZ)

(21) Appl. No.: 18/683,180

(22) PCT Filed: Aug. 16, 2022
(86) PCT No.: PCT/EP2022/072845

§ 371 Date: Feb. 12, 2024

(30) Foreign Application Priority Data
Aug. 13, 2021 (EP) 21191347.0

(51) Int. Cl. A61K 38/20; A61K 9/00; A61K 38/17; A61K 45/06; A61K 47/68; A61P 35/00; C07K 16/28

(52) U.S. Cl. CPC A61K 38/2086; A61K 9/0019; A61K 38/1793; A61K 45/06; A61K 47/6855; A61P 35/00; C07K 16/2818; C07K 16/2827

(57) ABSTRACT

The invention relates to an interleukin-2/interleukin-receptor βγ(IL-2/IL-15Rβγ) agonist for use in treating cancer in a patient, wherein said IL-2/IL-15Rβγ agonist is administered in combination with a cytotoxic compound capable of inducing immunogenic cell death (TCD) or in combination with an application of a modality capable of inducing TCD.

### BACKGROUND OF THE INVENTION

The past decade has seen significant advances in new cancer treatments through the development of highly selective small molecules that target a specific genetic abnormality responsible for the disease (Weinstein 2005, McDermott and Settleman 2009). Although this approach has seen great success in application to malignancies with a single, well-defined oncolytic driver, resistance is commonly observed in more complex cancer settings (Rosenzweig 2012, Giroux 2013). Traditional cytotoxic agents are another approach to treating cancer; however, unlike target-specific approaches, they suffer from adverse effects stemming from nonspecific killing of both healthy and cancer cells. A strategy that combines the powerful cell-killing ability of potent cytotoxic agents with target specificity would represent a potentially new paradigm in cancer treatment. Antibody-drug-conjugates (ADCs) are such an approach, wherein the antibody component provides specificity for a tumor target antigen and the drug confers the cytotoxicity. Recent progress in ADC technology together with further development of modalities for antibody-mediated targeting, such as immunotoxins, immunoliposomes and radionuclide conjugates represents the next wave of cancer therapeutics.

On the contrary, lack of therapeutic potential or safety considerations resulted in the fact that currently only four ADCs received the US Food and Drug Administration (FDA) approval to be used in the treatment of cancer with only one being approved for the treatment of solid tumors. Ado-trastuzumab emtansine (T-DM1, Kadcyla®), a HER2 targeting ADC combining the humanized antibody trastuzumab with a potent anti-microtubule cytotoxic agent emtansine, a derivative of maytansine (DM1), was approved for the treatment of patients with HER2-positive breast cancer (LoRusso, Weiss et al. 2011, Verma, Miles et al. 2012). It has been shown though that the therapeutic effect of T-DM1 is fully dependent on the HER2 expression and eligible for the treatment are only patients with tumor positive at a level of 3+ immunohistochemistry (IHC) by Dako Herceptest™ or FISH amplification ratio ≥2.0 by Dako HER2 FISH PharmDx™ test kit. There are almost 30 ADCs in advanced stages of clinical development, some of them already indicating higher therapeutic potential than T-DM1. On the one hand, due to safety considerations many of these ADCs under development are based on toxins with a comparably low potency, which in turn can lead to a significantly decreased antitumor efficacy especially in tumors with low to intermediate target expression. On the other hand, there are several ADCs in development which are loaded with highly potent toxins that are expected be highly efficacious, but having—despite targeting—also high off-target toxicities resulting in a limited therapeutic window. In order to overcome this dilemma and broaden the therapeutic potential of this promising class of drugs, novel concepts are needed to increase their therapeutic window and/or decreasing number and severity of severe adverse events.

### SUMMARY OF THE INVENTION

The inventors have surprisingly found, that the combination of a certain class of ADCs, capable of inducing immunogenic cell death, in combination with the emerging class of interleukin-2/interleukin-15 receptor βγ (IL-2/IL-15Rβγ) agonists results in improved antitumor efficacy. Accordingly, the present invention provides an IL-2/IL-15Rβγ agonist for use in treating cancer in patient, wherein said IL-2/IL-15Rβγ agonist, (a) is administered simultaneously with or sequentially to a cytotoxic compound capable of inducing immunogenic cell death (ICD), (b) is administered simultaneously with or sequentially to applying a modality capable of inducing ICD, (c) is administered simultaneously with a cytotoxic compound capable of inducing ICD and simultaneously with a modality capable of inducing ICD, (d) is administered simultaneously with a cytotoxic compound capable of inducing ICD and sequentially to a modality capable of inducing ICD, (e) is administered sequentially to a cytotoxic compound capable of inducing ICD and simultaneously with a modality capable of inducing ICD, or (f) is administered sequentially to a cytotoxic compound capable of inducing ICD and sequentially to a modality capable of inducing ICD.

The concept of immunogenic cell death (ICD), its induction and related therapeutic benefits provide a rationale for the development of various therapeutic agents and modalities. ICD is a specific cell death modality occurring in a defined temporal sequence, stimulating an immune response against dead-cell antigens (Kroemer. Galluzzi et al. 2013) characterized by the early surface exposure of chaperones including calreticulin (CRT) and heat shock proteins (HSPs, e.g. HSP70 and HSP90). This affects dendritic cell maturation, the uptake and presentation of tumor antigens as well as the late release of soluble mediators like HMGB1, which, through TLR4, augments the presentation of antigens from dying tumor cells to dendritic cells (Fucikova, Kralikova et al. 2011). Such signals operate on a series of receptors expressed by dendritic cells. ICD is believed to be a prominent pathway for the activation of the immune system against cancer, and the understanding of its underlying mechanisms may facilitate the design of highly efficient anticancer treatments, whereas suboptimal regimens (failing to induce TCD), selective alterations in cancer cells (preventing the emission of immunogenic signals during ICD), or defects in immune effectors (abolishing the perception of ICD by the immune system) can all contribute to therapeutic failure (Kroemer, Galluzzi et al. 2013).

On the other hand, immunotherapies, i.e., treatments that make use of the body's own immune system to help fighting the disease, aim at harnessing the power of the immune system to kill malignant tumor cells or infected cells, while leaving healthy tissues intact. Whereas the immune system has an inherent ability to find and eliminate malignancies, tumors and persistent infections have developed mechanisms to escape immune surveillance (Robinson and Schluns 2017). The potential reasons for immune tolerance include failed innate immune activation, the involvement of dense stroma as a physical barrier, and a possible contribution of immune suppressive oncogene pathways (Gajewski, Woo et al. 2013). One group of immunotherapies with some clinical success are cytokine treatments, more specifically interleukin 2 (IL-2), commercially available as aldesleukin/PROLEUKIN® (Prometheus Laboratories Inc.) and interleukin 15 (IL-15) therapies known to activate both the innate immune response through NK cells and the adaptive immune response through CD8+ T cells (Steel, Waldmann et al. 2012, Conlon, Miljkovic et al. 2019). While impressive tumor regression was observed with IL-2 therapy, responses are limited to small percentages of patients and carry with it a high level of even life-threatening toxicity. Further, IL-2 displayed not only immune-enhancing but also immune-suppressive activities through the induction of activation-induced cell death of T cells and the expansion of immunosuppressive regulatory T cells (T_{regs}) (Robinson and Schluns 2017).

Both IL-2 and IL-15 act through heterotrimeric receptors having α, β and γ subunits, whereas they share the common gamma-chain receptor (γ_{c} or γ, CD132)—also shared with IL-4, IL-7, IL-9 and IL-21—and the IL-2/IL-15RP (also known as IL-2RP, CD122). As a third subunit, the heterotrimeric receptors contain a specific subunit for IL-2 or IL-15, i.e., the IL-2Rα (CD25) or the IL-15Rα (CD215). Downstream, IL-2 and IL-15 heterotrimeric receptors share JAK1 (Janus kinase 1), JAK 3 and STAT3/5 (signal transducer and activator of transcription 3 and 5) molecules for intracellular signaling leading to similar functions, but both cytokines also have distinct roles as reviewed in Waldmann (2015, see e.g. table 1) and Conlon (2019). Accordingly, the activation of different heterotrimeric receptors by binding of IL-2, IL-15 or derivatives thereof potentially leads to a specific modulation of the immune system and potential side effects. Recently, novel compounds were designed aiming at specifically targeting the activation of NK cells and CD8⁺ T cells.

These are compounds targeting the mid-affinity IL-2/IL-15Rβγ, i.e., the receptor consisting of the IL-2/IL-15Rβ and the γ_{c} subunits, which is expressed on NK cells, CD8⁺ T cells, NKT cells and γδ T cells. This is critical for safe and potent immune stimulation mediated by IL-15 trans-presentation, whereas the designed compounds RLI-15, ALT-803 and hetIL-15 already contain (part of) the IL-15Rα subunit and therefore simulate trans-presentation of the α subunit by antigen presenting cells. RLI-15 binds to the mid-affinity IL-15Rβγ only, as it comprises the covalently attached sushi+ domain of IL-15Rα. In turn, RLI-15 binds neither to IL-15Rα nor to IL-2Rα. Similarly, ALT-803 and hetIL-15 (NIZ985) carry an IL-15Rα sushi domain or the soluble IL-15Rα, respectively, and therefore bind to the mid-affinity IL-15Rβγ receptor. However, due to their non-covalent binding there is a chance that the complex dissociates in vivo and thereby the dissociated fraction of the applied complex further exerts other binding (see below). Probability for dissociation is likely higher for ALT-803 vs. hetIL-15, as ALT-803 only comprises the sushi domain of IL-15Rα, which is known to mediate only partial binding to IL-15, whereas the sushi+ domain is required for full binding (Wei, Orchardson et al. 2001). Other examples for complexes of IL-15 and IL-15Rα in various formats are XmAb24306 (WO2014/145806A2), P-22339 (U.S. Pat. No. 10,206,980), CUG105 (WO2019/246379A1).

Another example of targeting mid-affinity IL-2/IL-15Rβγ receptors is PEGylated IL-2, with the example NKTR-214, whose hydrolyzation to its most active 1-PEG-IL-2 state generates a species whose location of PEG chains at the IL-2/IL-2Rα interface interferes with binding to the high-affinity IL-2Rα, while leaving binding to the mid-affinity IL-2/IL-15Rβ unperturbed (Charych, Hoch et al. 2016). Further, THOR-707 is a site-directed, singly PEGylated form of IL-2 with reduced/lacking IL2Rα chain engagement while retaining binding to the intermediate affinity IL-2Rβγ signaling complex (Joseph, Ma et al. 2019) (WO2019/028419A1). Also, the IL-2/IL-2Rα fusion protein ALKS 4230 comprising a circularly permutated (to avoid interaction of the linker with the β and γ receptor chains) IL-2 with the extracellular domain of IL-2Rα selectively targets the βγ receptor as the α-binding side is already occupied by the IL-2Rα fusion component (Lopes, Fisher et al. 2020). Further pegylated IL-2-based therapeutics specific for the IL-2/IL-25Rβγ are TransCon IL-2 (Rosen, Kvarnhammar et al. 2022) (WO2019/7185705 and WO 2021/7245130) and ARX102 (WO2020/056066, WO2021183832).

Further, the IL-2 mutant IL2v with abolished binding to the IL-2Rα subunit is an example of this class of compounds (Klein, Inja et al. 2013, Bacac, Fauti et al. 2016), as well as NL-201, which mimics IL-2 to bind to the IL-2 receptor βγ_{c} heterodimer (IL-2Rβγ_{c}) but has no binding site for IL-2Rα or IL-15Rα (Silva, Yu et al. 2019). Other IL-2/IL-25Rβγ selective IL-2 muteins are STK-012 (Sockolosky, Trotta et al. 2018, Mendoza, Escalante et al. 2019) (WO2019/113221) and MDNA11 (Merchant, Galligan et al. 2022) (WO2018/234862).

In addition, conditionally activated IL-2 derivatives have been developed, e.g., WTX-124 (Silva 2022) (WO2020/232305) and XTX202 (O'Neil, Guzman et al. 2021, abstract and poster) (WO2020/069398).

Another strategy to target the IL-2/IL-15Rβ receptors is the use of IL-15 muteins, which have a decreased or no binding to the IL-15Rα (WO 2019/166946A1), thereby reducing or avoiding completely the activation of the high affinity IL-15Rαβγ receptor. Similarly, IL-15 is PEGylated in order to reduce the binding to the IL-15Rα while retaining the binding to the IL-2/IL-15βγ receptor, e.g., NKRT-255 (WO2018/213341A1) and THOR-924, -908, -918 (WO2019/165453A1). In WO2016/060996A2 PEGylation for half-life extension is combined with mutating IL-15.

This class of compounds, by targeting of the mid-affinity IL-2/IL-15Rβγ receptors, avoids liabilities associated with targeting the high-affinity IL-2 and IL-15 receptors such as T_{reg} activation induced by IL-2 or vascular leakage syndrome which can be induced by high concentrations of soluble IL-2 or IL-15. This is due to the fact that the IL-2Rαβγ high affinity receptor is additionally expressed on CD4⁺ T_{regs} and vascular endothelium, and is activated by IL-2 cis-presentation. Therefore, compounds targeting (also) the high-affinity IL-2Rαβγ potentially lead to T_{reg} expansion and vascular leak syndrome (VLS), as observed for native IL-2 or soluble IL-15 (Conlon, Miljkovic et al. 2019). Potentially VLS can be also caused by the de-PEGylated NKTR-214. De-PEGylated NKT2-214 has however a short half-life and it needs to be seen in the clinical development whether at all or to which extent this side-effect plays a role.

The high-affinity IL-15Rαβγ receptors activated by IL-15 cis-presentation are constitutively expressed in T cell leukemia and upregulated on inflammatory NK cells, inflammatory CD8⁺ T cells and Fibroblast-like synoviocytes (Kurowska, Rudnicka et al. 2002, Perdreau, Mortier et al. 2010), i.e. these cells also express the IL-15Rα subunit. Such activation should be avoided because of the IL-15 cis-presentation on these cells is involved in the development of T cell leukemia and exacerbation of the immune response, potentially triggering autoimmune diseases. Similarly, the high-affinity IL-15Rαβγ receptor is expressed on vascular endothelium and soluble IL-15 can also induce VLS. IL-15/IL-15Rα complexes, and similarly other compounds targeting the IL-2/IL-15Rβγ receptors described above, do not bind to this high-affinity receptor as they already carry at least the sushi domain of the IL-15Rα, which sterically hinders the binding to the heterotrimeric IL-15Rαβγ receptor, or binding to the IL-15Rα is reduced/abolished by mutation, or sterically hindered by fusion to other moieties such as PEG, albumin. These side effects triggered via engagement of high affinity IL-15Rαβγ receptors are triggered by native IL-15, but also by non-covalent IL-15/IL-15Rα complexes such as ALT-803 and hetIL-15, if disintegration of the complexes occurs in vivo.

Finally, the high-affinity IL-15Rα is constitutively expressed on myeloid cells, macrophages, B cells and neutrophils (Chenoweth, Mian et al. 2012) and may be activated by native IL-15 and again by non-covalent IL-15/IL-15Rα complexes such as ALT-803 and hetIL-15, if disintegration of the complexes occurs in vivo.

In analogy, also the above described IL-2 based compound targeting the joined IL-2/IL-15Rβγ function through reducing/abolishing binding by mutation (STK-012, MDNA11), sterically hindering the binding to IL-2Rα by fusion to the soluble IL-2Rα (ALKS4230) or to other moieties such as PEG (NKTR-214, SAR245) to avoid the life-threatening side effects of IL-2.

In summary, IL-15 has similar immune enhancing properties as wildtype IL-2, but it is believed to not share the immune-suppressive activities like activation of T_{reg} cells and does not cause VLS in the clinic (Robinson and Schluns 2017), whereas drawbacks of IL-15 treatment include its short in vivo half-life and its reliance on trans-presentation by other cell types (Robinson and Schluns 2017). Both the IL-15 therapies and the improved IL-2 therapies target the same, mid-affinity IL-2/IL-15Rβγ and at the same time detargeting from the respective α-chains, thereby forming a group of similar acting compounds, the IL-2/IL-15Rβγ agonists.

In the recent years, these findings led to a growing number of engineered IL-2/IL-15Rβγ agonists (some of them mentioned above), and some of them recently entered clinical development. This list of IL-2/IL-15Rβγ agonists includes RLI-15 (SOT101, SO-C101), ALT-803 (N803, Anktiva), hetIL-15 (NIZ985), XmAb24306, P-22339, CUG105, NKTR-214, SAR245 (THOR-707), Nemvaleukin alpha (ALKS4230), NL-201, NKRT-255, THOR-924, TransCon IL-2, ARX102, STK-012, MDNA11, WTX-124, XTX202, NKRT-255 and THOR-924, -908, -918.

As shown by the examples below, the stimulation of the immune system by the IL-2/IL-15Rβγ agonist RLI-15 in combination the ADC T-DM1 lead to synergistic tumor cell killing in vivo and in combination with SOT102 (with PNU as a toxin) in vitro. Allegedly, without being bound to such mechanism, T-DM1 and PNU induce ICD thereby priming dendritic cells against the dying tumor cells and/or upregulating NK-cell receptors on the tumor cells. However, it required the additional stimulation of immune cells like NK cells and CD8⁺ cells by RLI-15 (or another IL-2/IL-15Rβγ agonist) to result in a superior/synergistic tumor cell killing.

### Definitions, Abbreviations and Acronyms

“Antibodies” or “antibody”, also called “immunoglobulins” (Ig), generally comprise four polypeptide chains, two heavy (H) chains and two light (L) chains, and are therefore multimeric proteins, or comprise an equivalent Ig homologue thereof (e.g., a camelid antibody comprising only a heavy chain, single-domain antibodies (sdAb) or nanobodies which can either be derived from a heavy or a light chain). The term “antibodies” includes antibody-based binding proteins, modified antibody formats retaining their target binding capacity. The term “antibodies” also includes full length functional mutants, variants, or derivatives thereof (including, but not limited to, murine, chimeric, humanized and fully human antibodies) which retain the essential epitope binding features of an Ig molecule, and includes dual specific, bispecific, multispecific, and dual variable domain Igs. Ig molecules can be of any class (e.g., IgG, IgE, IgM, IgD, IgA, and IgY), or subclass (e.g., IgG1, IgG2, IgG3, IgG4, IgA1, and IgA2) and allotype. Ig molecules may also be mutated e.g. to enhance or reduce affinity for Fcγ receptors or the neonatal Fc receptor (FcRn) or other known reason.

An “antibody fragment” or “antibody binding fragment”, as used herein, relates to a molecule comprising at least one polypeptide chain derived from an antibody that is not full length and exhibits target binding, including, but not limited to (i) a Fab fragment, which is a monovalent fragment consisting of the variable light (VL), variable heavy (VH), constant light (CL) and constant heavy 1 (CH1) domains; (ii) a F(ab′)₂ fragment, which is a bivalent fragment comprising two Fab fragments linked by a disulfide bridge at the hinge region (reduction of a F(ab′)₂ fragment result in two Fab′ fragment with a free sulfhydryl group); (iii) a heavy chain portion of a Fab (Fa) fragment, which consists of the VH and CH1 domains; (iv) a variable fragment (Fv) fragment, which consists of the VL and VH domains of a single arm of an antibody; (v) a domain antibody (dAb) fragment, which comprises a single variable domain; (vi) an isolated complementarity determining region (CDR); (vii) a single chain Fv fragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fv segments (VH-CH1-VH-CH1) which, together with complementarity light chain polypeptides, form a pair of antigen binding regions; (x) Dual-Variable Domain Immunoglobulin (xi) other non-full length portions of immunoglobulin heavy and/or light chains, or mutants, variants, or derivatives thereof, alone or in any combination. Engineered antibody variants are reviewed in Holliger and Hudson, and Friedman (Holliger and Hudson 2005, Friedman and Stahl 2009). An antibody fragment retains at least some of the binding specificity of the parental antibody, typically at least 10% of the parental binding activity when that activity is expressed on a molar basis. Given the high affinity/avidity of antibodies, even 10% of the parental binding activity is typically sufficient to exert its action and/or such reduction of binding activity could easily be compensated by higher dosing. Preferably, an antibody fragment retains at least 20%, 50%, 70%, 80%, 90%, 95% or 100% or more, especially at least 90%, of the parental antibody's binding affinity for the target.

The term “modified antibody format”, as used herein, encompasses polyalkylene oxide-modified scFv, monobodies, diabodies, camelid antibodies, domain antibodies, bi- or trispecific antibodies, IgA, or two IgG structures joined by a J chain and a secretory component, shark antibodies, new world primate framework and non-new world primate CDR, IgG4 antibodies with hinge region removed, IgG with two additional binding sites engineered into the CH3 domains, antibodies with altered Fc region to enhance or reduce affinity for Fc gamma receptors, dimerized constructs comprising CH3, VL, and VH, and the like. Bispecific antibody formats are for example reviewed in Godar et al. (2018).

The Kabat numbering scheme Martin and Allemn (2014) has been applied to the disclosed antibodies.

“Antibody-drug conjugate” or “ADC”, as used herein, refers to an antibody (or antibody fragments) to which a pharmaceutically active ingredient (API), or payload, has been covalently coupled, such that the API is targeted by the antibody to the target of the antibody to exhibit its pharmaceutical function primarily in cells expressing the target of the antibody. Typically, the API is a cytotoxic drug or toxin able to effectively kill cells expressing the target. The covalent coupling of the API can be performed in a non-site specific manner using standard chemical linkers that couple the API to lysine or cysteine residues of the antibody, or preferably in a site specific manner by mechanisms e.g. reviewed in Panowski, Bhakta et al. (2014), whereas using sortase mediated transpeptidation is preferred (as described in WO 2014/140317A2). Used linkers are required to have discrete properties as reviewed by Jain, Smith et al. (2015), such as being stable in plasma, but liberating the API upon internalization by the (target) cell, and at the same time increase the solubility to avoid aggregation of the typically hydrophobic APIs used for ADCs and having no or low immunogenicity. Linkers may be cleavable upon binding to the target or in the microtumor environment in order to increase the by-stander effect, or non-cleavable linkers to ensure liberation of the API as much as possible to the interior of the (target) cell. One important feature of ADCs is the averaged ration of covalently linked API (drug) to antibody, the so-called drug-antibody-ratio (DAR), where typically a low variability for a medicinal product is preferred and a DAR 2 to 4 (i.e. 2 to 4 APIs coupled to one antibody) is targeted.

“Immunogenic cell death” or “ICD”, as used herein, refers to a cell death modality that stimulates an immune response against dead-cell antigens (e.g. cancer cells) showing distinct biochemical properties (“ICD markers”) including the exposure of the so-called DAMPs (Danger-Associated Molecular Patterns) represented mainly by cell surface exposure of calreticulin (CRT), Heat-shock protein 70 and 90, secretion of ATP, and release of nonhistone chromatin protein high-mobility group box 1 (HMGB1) (Kroemer, Galluzzi et al. 2013). These markers of ICD can easily be determined as described in the examples, especially as in Example.

“Cytotoxic compound capable of inducing immunogenic cell death (ICD)” or “ICD inducing compounds” are generally compounds or agents which, upon incubation, induce ICD as measurable in vitro by induction of expression of ICD markers on cell lines, especially tumor cell lines, by apoptotic, annexin V-positive/DAPI-negative cells, preferably to a similar extent as doxorubicin or idarubicin as described by Fucikova et al. (2011, 2014), whereas cytotoxic compounds preferably are small molecules of a size below about 1000 Dalton that can easily enter cells due to their low molecular weight being cytotoxic, i.e. being toxic to cells.

“Modality capable of inducing ICD” are generally treatment modalities which, upon subjecting tumor cell lines to such modality, induce ICD as measurable in vitro by induction of expression of ICD markers on cell lines, especially tumor cell lines, by apoptotic, annexin V-positive/DAPI-negative cells, preferably to a similar extent as doxorubicin or idarubicin as described by Fucikova et al. (Fucikova, Kralikova et al. 2011, 2014).

“SOT102” is an antibody-drug-conjugate based on the anti-CLDN18.2 antibody hCl1a (SEQ ID NO: 20 (heavy chain), SEQ ID NO: 21 (light chain)) having the ADCC inactivating heavy chain substitutions LALA (L234A|L235A) with the anthracycline PNU-159682 (PNU) linked to the C-terminus of the light chains by the non-cleavable linker GGGGSLPQTGG (SEQ ID NO: 24)-ethylenediamine (hCl1a-LC-G2-PNU). The preparation of SOT102 is described in Example 7 of WO 2022/136642. SEQ ID NO: 22 and SEQ ID NO: 23 involve the LALA mutation and the non-cleavable liker.

“Treating” in connection with a disease means providing medical care to a patient including curative, palliative or prophylactic treatment.

“Low to intermediate HER2 expression” means HER2 expression as measured by HercepTest™ having a HER2 protein expression score of 0 to 2+, preferably 0 to 1+ in surgical specimens or biopsy specimens, i.e. comparable to expression levels comparable to MDA-231 to MDA-175 control slides of the HercepTest™, which is a semi-quantitative immunohistochemical assay to determine HER2 protein overexpression in breast cancer tissues routinely processed for histological evaluation comparing to included control slides representing different levels of HER2 protein expression: MDA-231 (0), MDA-175 (1+) and SK-BR-3 (3+). HER2 3+ refers to high HER2 expression.

“Interleukin-2”, “IL-2” or “IL2” refers to the human cytokine as described by NCBI Reference Sequence AAB46883.1 or UniProt ID P60568 (SEQ ID NO: 1). Its precursor protein has 153 amino acids, having a 20-aa peptide leader and resulting in a 133-aa mature protein. Its mRNA is described by NCBI GenBank Reference S82692.1.

“IL-2 derivative” refers to a protein having a percentage of identity of at least 92%, preferably of at least 96%, more preferably of at least 98%, and most preferably of at least 99% with the amino acid sequence of the mature human IL-2 (SEQ ID NO: 2). Preferably, an IL-2 derivative has at least about 0.1% of the activity of human IL-2, preferably at least 1%, more preferably at least 10%, more preferably at least 25%, even more preferably at least 50%, and most preferably at least 80%, as determined by a lymphocyte proliferation bioassay. As interleukins are extremely potent molecules, even such low activities as 0.1% of human IL-2 may still be sufficiently potent, especially if dosed higher or if an extended half-life compensates for the loss of activity. Its activity is expresses in International Units as established by the World Health Organization 1^{st} International Standard for Interleukin-2 (human), replaced by the 2^{nd} International Standard (Gearing and Thorpe 1988, Wadhwa, Bird et al. 2013). The relationship between potency and protein mass is as follows: 18 million IU PROLEUKIN=1.1 mg protein. As described above, mutations (substitutions) may be introduced in order to specifically link PEG to IL-2 for extending the half-life as done for THOR-707 (Joseph, Ma et al. 2019) (WO2019/028419A1) or for modifying the binding properties of the molecule, e.g. reduce the binding to the IL-2a receptor as done for IL2v (Klein, Inja et al. 2013, Bacac, Fauti et al. 2016) (WO2012/107417A1) by mutation of L72, F42 and/or Y45, especially F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, preferably mutations F42A, Y45A and L72G. Various other mutations of IL-2 have been described: R38W for reducing toxicity (Hu, Mizokami et al. 2003) due to reduction of the vasopermeability activity (US 2003/0124678); N88R for enhancing selectivity for T cells over NK cells (Shanafelt, Lin et al. 2000); R38A and F42K for reducing the secretion of proinflammatory cytokines from NK cells ((Heaton, Ju et al. 1993) (U.S. Pat. No. 5,229,109); D20T, N88R and Q126D for reducing VLS (US 2007/0036752); R38W and F42K for reducing interaction with CD25 and activation of T_{reg} cells for enhancing efficacy (WO2008/003473); and additional mutations may be introduced such as T3A for avoiding aggregation and C125A for abolishing O-glycosylation (Klein, Waldhauer et al. 2017). Other mutations or combinations of the above may be generated by genetic engineering methods and are well known in the art. Amino acid numbers refer to the mature IL-2 sequence of 133 amino acids (SEQ ID NO: 2).

“Interleukin-15”, “IL-15” or “IL15” refers to the human cytokine as described by NCBI Reference Sequence NP_000576.1 or UniProt ID P40933 (SEQ ID NO: 3). Its precursor protein has 162 amino acids, having a long 48-aa peptide leader and resulting in a 114-aa mature protein (SEQ ID NO: 4). Its mRNA, complete coding sequence is described by NCBI GenBank Reference U14407.1.

“IL-15 derivative” or “derivative of IL-15” refers to a protein having a percentage of identity of at least 92%, preferably of at least 96%, more preferably of at least 98%, and most preferably of at least 99% with the amino acid sequence of the mature human IL-15 (114 aa) (SEQ ID NO: 4). Preferably, an IL-15 derivative has at least 0.1% of the activity of human IL-15, preferably 1%, more preferably at least 10%, more preferably at least 25%, even more preferably at least 50%, and most preferably at least 80%. As for IL-2 described above, interleukins are extremely potent molecules, even such low activities as 0.1% of human IL-15 may still be sufficiently potent, especially if dosed higher or if an extended half-life compensates for the loss of activity. Also for IL-15, a plethora of mutations has been described in order to achieve various defined changes to the molecule: D8N, D8A, D61A, N65D, N65A, Q108R for reducing binding to the IL-15Rβγβγ_{c} receptors (WO 2008/143794A1); N72D as an activating mutation (in ALT-803); N1D, N4D, D8N, D30N, D61N, E64Q, N65D, and Q108E to reduce the proliferative activity (US 2018/0118805); L44D, E46K, L47D, V49D, I50D, L66D, L66E, I67D, and 167E for reducing binding to the IL-15Rα (WO 2016/142314A1); N65K and L69R for abrogating the binding of IL-15Rb (WO 2014/207173A1); Q101D and Q108D for inhibiting the function of IL-15 (WO 2006/020849A2); S7Y, S7A, K10A, K11A for reducing IL-15Rβ binding (Ring, Lin et al. 2012); L45, S51, L52 substituted by D, E, K or R and E64, I68, L69 and N65 replaced by D, E, R or K for increasing the binding to the IL-15Rα (WO 2005/085282A1); N71 is replaced by S, A or N, N72 by S, A or N, N77 by Q, S, K, A or E and N78 by S, A or G for reducing deamidation (WO 2009/135031A1); WO 2016/060996A2 defines specific regions of IL-15 as being suitable for substitutions (see para. 0020, 0035, 00120 and 00130) and specifically provides guidance how to identify potential substitutions for providing an anchor for a PEG or other modifications (see para. 0021); Q108D with increased affinity for CD122 and impaired recruitment of CD132 for inhibiting IL-2 and IL-15 effector functions and N65K for abrogating CD122 affinity (WO 2017/046200A1); N1D, N4D, D8N, D30N, D61N, E64Q, N65D, and Q108E for gradually reducing the activity of the respective IL-15/IL-15Rα complex regarding activating of NK cells and CD8 T cells (see FIG. 51, WO 2018/071918A1, WO 2018/071919A1). Additionally or alternatively, the artisan can easily make conservative amino acid substitutions. IL-15 derivatives may further be generated by chemical modification as known in the art, e.g. by PEGylation or other posttranslational modifications (see WO 2016/060996A2, WO 2017/112528A2, WO 2009/135031A1).

The activity of both IL-2 and IL-15 can be determined by induction of proliferation of kit225 cells as described by Hori et al. (1987). Preferably, methods such as colorimetry or fluorescence are used to determine proliferation activation due to IL-2 or IL-15 stimulation, as for example described by Soman et al. using CTLL-2 cells (Soman, Yang et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

“IL-2Rα” refers to the human IL-2 receptor α or CD25.

“IL-15Rα” refers to the human IL-15 receptor α or CD215 as described by NCBI Reference Sequence AAI21142.1 or UniProt ID Q13261 (SEQ ID NO: 5). Its precursor protein has 267 amino acids, having a 30-aa peptide leader and resulting in a 231-aa mature protein. Its mRNA is described by NCBI GenBank Reference HQ401283.1. The IL-15Rα sushi domain (or IL-15Rαₛᵤₛₕᵢ, SEQ ID NO: 6) is the domain of IL-15Rα which is essential for binding to IL-15 (Wei, Orchardson et al. 2001). The sushi+ fragment (SEQ ID NO: 7) comprising the sushi domain and part of the hinge region, defined as the fourteen amino acids which are located after the sushi domain of this IL-15Rα, in a C-terminal position relative to said sushi domain, i.e., said IL-15Rα hinge region begins at the first amino acid after said (C4) cysteine residue, and ends at the fourteenth amino acid (counting in the standard “from N-terminal to C-terminal” orientation). The sushi+ fragment reconstitutes full binding activity to IL-15 (WO 2007/046006).

“IL-15Rα derivative” refers to a polypeptide comprising an amino acid sequence having a percentage of identity of at least 92%, preferably of at least 96%, more preferably of at least 98%, and even more preferably of at least 99%, and most preferably 100% identical with the amino acid sequence of the sushi domain of human IL-15Rα (SEQ ID NO: 6) and, preferably of the sushi+ domain of human IL-15Rα (SEQ ID NO: 7). Preferably, the IL-15Rα derivative is a N- and C-terminally truncated polypeptide, whereas the signal peptide (amino acids 1-30 of SEQ ID NO: 5) is deleted and the transmembrane domain and the intracytoplasmic part of IL-15Rα is deleted (amino acids 210 to 267 of SEQ ID NO: 5). Accordingly, preferred IL-15Rα derivatives comprise at least the sushi domain (aa 33-93 but do not extend beyond the extracellular part of the mature IL-15Rα being amino acids 31-209 of SEQ ID NO: 5. Specific preferred IL-15Rα derivatives are the sushi domain of IL-15Rα (SEQ ID NO: 6), the sushi+ domain of IL-15Rα (SEQ ID NO: 7) and a soluble form of IL-15Rα (from amino acids 31 to either of amino acids 172, 197, 198, 199, 200, 201, 202, 203, 204 or 205 of SEQ ID NO: 5, see WO 2014/066527, (Giron-Michel, Giuliani et al. 2005)). Within the limits provided by this definition, the IL-15Rα derivative may include natural occurring or introduced mutations. Natural variants and alternative sequences are e.g. described in the UniProtKB entry Q13261 (www.uniprot.org/uniprot/Q13261). Further, the artisan can easily identify less conserved amino acids between mammalian IL-15Rα homologs or even primate IL-15Rα homologs in order to generate derivatives which are still functional. Respective sequences of mammalian IL-15Rα homologs are described in WO 2007/046006, page 18 and 19. Additionally or alternatively, the artisan can easily make conservative amino acid substitutions.

Preferably, an IL-15Rα derivative has at least 10% of the binding activity of the human sushi domain to human IL-15, e.g. as determined in Wei, Orchardson et al. (2001), more preferably at least 25%, even more preferably at least 50%, and most preferably at least 80%.

“IL-2Rβ” refers to the human IL-Rβ or CD122.

“IL-2Rγ” refers to the common human cytokine receptor γ or γ_{c} or CD132, shared by IL-4, IL-7, IL-9, IL-15 and IL-21.

An IL-15/IL-15Rα complex refers to a covalent or non-covalent complex comprising a human IL-15 or an IL-15 derivative and a human IL-15Rα or an IL-15Rα derivative. Preferably, the complex comprises human IL-15 and the sushi domain of IL-15Rα (SEQ ID NO: 6), the sushi+ domain of IL-15Rα (SEQ ID NO: 7) or a soluble form of IL-15Rα (from amino acids 31 to either of amino acids 172, 197, 198, 199, 200, 201, 202, 203, 204 or 205 of SEQ ID NO: 5, see WO 2014/066527, (Giron-Michel, Giuliani et al. 2005)).

“RLI-15” refers to an IL-15/IL-15Rα complex being a receptor-linker-interleukin (from N- to C-terminus; “RLI”) fusion protein of the human IL-15Rα sushi+ fragment with the human IL-15. Suitable linkers are flexible with low immunogenicity; examples are described in WO 2007/046006 and WO 2012/175222. The sushi domain or fragment of human IL-15Rα has the sequence as described by SEQ ID NO: 6 from the first to the fourth conserved cysteine, optionally extended N-terminally by T or IT and C-terminally by I. The sushi+ fragment of human IL-15Rα has the sequence as described by SEQ ID NO: 7, which additionally comprises part of the hinge region and exerts.

“RLI2” or “SO-C101” or “SOT101” refer to an IL-15/IL-15Rα complex being a receptor-linker-interleukin fusion protein of the human IL-15Rα sushi+ fragment with the human IL-15. “RLI2” or “SO-C101” or “SOT101” are represented by SEQ ID NO: 9. The linker used in “RLI2” or “SO-C101” or “SOT101” has the sequence of SEQ ID NO: 8.

“ALT-803” refers to an IL-15/IL-15Rα complex of Altor BioScience Corp., which is a complex containing 2 molecules of an optimized amino acid-substituted (N72D) human IL-15 “superagonist”, 2 molecules of the human IL-15a receptor “sushi” domain fused to a dimeric human IgG1 Fc that confers stability and prolongs the half-life of the IL-15_{N72D}:IL-15Rαₛᵤₛₕᵢ-Fc complex (see for example US 2017/0088597).

“Heterodimeric IL-15:IL-Ra”, “hetIL-15” or “NIZ985” refer to an IL-15/IL-15Rα complex of Novartis which resembles the IL-15, which circulates as a stable molecular complex with the soluble IL-15Rα, which is a recombinantly co-expressed, non-covalent complex of human IL-15 and the soluble human IL-15Rα (sIL-15Rα), i.e. 170 amino acids of IL-15Rα without the signal peptide and the transmembrane and cytoplasmic domain (Thaysen-Andersen, Chertova et al. 2016, see e.g. table 1).

“IL-2/IL-15Rβγ agonists” refers to molecules or complexes which primarily bind to the mid-affinity IL-2/IL-15Rβγ receptor without binding/having widely reduced binding to the IL-2Rα and/or IL-15Rα receptor, thereby lacking/avoiding a stimulation of T_{regs}. “widely reduced binding” in this context means that binding is reduced by at least 50%, preferably at least 75%, especially by at least 90%. Examples are IL-15 bound to at least the sushi domain of the IL-15Rα having the advantage of not being dependent on trans-presentation or cell-cell interaction, and of a longer in vivo half-life due to the increased size of the molecule, which have been shown to be significantly more potent that native IL-15 in vitro and in vivo (Robinson and Schluns 2017). Besides IL-15/IL-15Rα based complexes, this can be achieved by mutated or chemically modified IL-2, which have a markedly reduced or timely delayed binding to the IL-2α receptor without affecting the binding to the IL-2/15Rβ and γ_{C} receptor or IL-15 muteins, as outlined above.

“NKTR-214” refers to an IL-2/IL-15Rβγ agonist based on IL-2, being a biologic prodrug consisting of IL-2 bound by 6 releasable polyethylene glycol (PEG) chains (WO 2012/065086A1). The presence of multiple PEG chains creates an inactive prodrug, which prevents rapid systemic immune activation upon administration. Use of releasable linkers allows PEG chains to slowly hydrolyze continuously forming active conjugated IL-2 bound by 2-PEGs or 1-PEG. The location of PEG chains at the IL-2/IL-2Rα interface interferes with binding to high-affinity IL-2Rα, while leaving binding to low-affinity IL-2RP unperturbed, favoring immune activation over suppression in the tumor (Charych, Hoch et al. 2016, Charych, Khalili et al. 2017).

THOR-707 refers to an IL-2/IL-15Rβγ agonist based on a site-directed, singly PEGylated form of IL-2 with reduced/lacking IL2Rα chain engagement while retaining binding to the intermediate affinity IL-2Rβγ signaling complex (Joseph, Ma et al. 2019) (WO 2019/028419A1).

ALKS 4230 refers to a circularly permutated (to avoid interaction of the linker with the β and γ receptor chains) IL-2 with the extracellular domain of IL-2Rα selectively targets the βγ receptor as the α-binding side is already occupied by the IL-2Rα fusion component (Lopes, Fisher et al. 2020).

NL-201 refers to IL-2/IL-15Rβγ agonists, which is are computationally designed protein that mimics IL-2 to bind to the IL-2 receptor βγ_{c} heterodimer (IL-2Rβγ_{c}) but has no binding site for IL-2Rα or IL-15Rα (Silva, Yu et al. 2019).

NKRT-255 refers to an IL-2/IL-15Rβγ agonist based on a PEG-conjugated human IL-15 that retains binding affinity to the IL-15Rα and exhibits reduced clearance to provide a sustained pharmacodynamic response (WO 2018/213341A1).

THOR-924, -908, -918 refer to IL-2/IL-15Rβγ agonists based on PEG-conjugated IL-15 with reduced binding to the IL-15Rα with a unnatural amino acid used for site-specific PEGylation (WO 2019/165453A1)

“IL2v” refers to an IL-2/IL-15Rβγ agonist based on IL-2, being an IL-2 variant with abolished binding to the IL-2Rα subunit with the SEQ ID NO: 10. IL2v is used for example in fusion proteins, fused to the C-terminus of an antibody. IL2v was designed by disrupting the binding capability to IL-2Rα through amino acid substitutions F42A, Y45A and L72G (conserved between human, mouse and non-human primates) as well as by abolishing O-glycosylation through amino acid substitution T3A and by avoidance of aggregation by a C125A mutation like in aldesleukin (numbering based on UniProt ID P60568 excluding the signal peptide) (Klein, Waldhauer et al. 2017). IL2v is used as a fusion partner with antibodies, e.g. with untargeted IgG (IgG-IL2v) in order to increase its half-life (Bacac, Colombetti et al. 2017). In RG7813 (or cergutuzumab amunaleukin, RO-6895882, CEA-IL2v) IL2v is fused to an antibody targeting carcinoembryonic antigen (CEA) with a heterodimeric Fc devoid of FcγR and C1q binding (Klein 2014, Bacac, Fauti et al. 2016, Klein, Waldhauer et al. 2017). And, in RG7461 (or R06874281 or FAP-IL2v) IL2v is fused to the tumor specific antibody targeting fibroblast activation protein-alpha (FAP) (Klein 2014).

“Immune check point inhibitor”, or in short “check point inhibitors”, refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2 (definition of the National Cancer Institute at the National Institute of Health, see www.cancer.gov/publications/dictionaries/cancer-terms/def/immune-checkpoint-inhibitor), as for example reviewed by Darvin et al. (2018). Examples of such check point inhibitors are anti-PD-L1 antibodies, anti-PD-1 antibodies, anti-CTLA-4 antibodies, but also antibodies against LAG-3 or TIM-3, or blocker of BTLA currently being tested in the clinic (De Sousa Linhares, Leitner et al. 2018). Further promising check point inhibitors are anti-TIGIT antibodies (Solomon and Garrido-Laguna 2018).

“anti-PD-L1 antibody” refers to an antibody, or an antibody fragment thereof, binding to PD-L1. Examples are avelumab, atezolizumab, durvalumab, KN035, MGD013 (bispecific for PD-1 and LAG-3).

“anti-PD-1 antibody” refers to an antibody, or an antibody fragment thereof, binding to PD-1. Examples are pembrolizumab, nivolumab, cemiplimab (REGN2810), BMS-936558, SHR1210, IBI308, PDR001, BGB-A317, BCD-100, JS001.

“anti-PD-L2 antibody” refers to an antibody, or an antibody fragment thereof, binding to anti-PD-L2. An example is sHIgM12.

“anti-CTLA4 antibody” refers to an antibody, or an antibody fragment thereof, binding to CTLA-4. Examples are ipilimumab and tremelimumab (ticilimumab).

“anti-LAG-3” antibody refers to an antibody, or an antibody fragment thereof, binding to LAG-3. Examples of anti-LAG-3 antibodies are relatlimab (BMS 986016), Sym022, REGN3767, TSR-033, GSK2831781, MGD013 (bispecific for PD-1 and LAG-3), LAG525 (IMP701).

“anti-TIM-3 antibody” refers to an antibody, or an antibody fragment thereof, binding to TIM-3. Examples are TSR-022 and Sym023.

“anti-TIGIT antibody” refers to an antibody, or an antibody fragment thereof, binding to TIGIT. Examples are tiragolumab (MTIG7192A, RG6058) and etigilimab (WO 2018/102536).

“Percentage of identity” or “% identical” between two amino acids sequences means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by Smith and Waterman (1981), by using the local homology algorithm developed by Needleman and Wunsch (1970), by using the method of similarities developed by Pearson and Lipman (1988), by using computer software using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar 2004), or by using CLUSTAL (Goujon, McWilliam et al. 2010). To get the best local alignment, one can preferably use the BLAST software with the BLOSUM 62 matrix. The identity percentage between two sequences of amino acids is determined by comparing these two sequences optimally aligned, the amino acids sequences being able to encompass additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

Conservative amino acid substitutions refers to a substation of an amino acid, where an aliphatic amino acid (i.e. Glycine, Alanine, Valine, Leucine, Isoleucine) is substituted by another aliphatic amino acid, a hydroxyl or sulfur/selenium-containing amino acid (i.e. Serine, Cysteine, Selenocysteine, Threonine, Methionine) is substituted by another hydroxyl or sulfur/selenium-containing amino acid, an aromatic amino acid (i.e. Phenylalanine, Tyrosine, Tryptophan) is substituted by another aromatic amino acid, a basic amino acid (i.e. Histidine, Lysine, Arginine) is substituted by another basic amino acid, or an acidic amino acid or its amide (Aspartate, Glutamate, Asparagine, Glutamine) is replaced by another acidic amino acid or its amide.

When it is stated “administered in combination” this typically does not mean that the two agents are co-formulated and co-administered, but rather one agent has a label that specifies its use in combination with the other. So, for example the IL-2/IL-15Rβγ agonist is for use wherein the use in treating or managing cancer or infectious diseases, comprising simultaneously, separately, or sequentially administering of the IL-2/IL-15Rβγ agonist and a further therapeutic agent, or vice e versa. But nothing in this application should exclude that the two combined agents are provided as a bundle or kit, or even are co-formulated and administered together where dosing schedules match. So, “administered in combination” includes (i) that the drugs are administered together in a joint infusion, in a joint injection or alike, (ii) that the drugs are administered separately but in parallel according to the given way of administration of each drug, and (iii) that the drugs are administered separately and sequentially.

Parallel administration in this context preferably means that both treatments are initiated together, e.g. the first administration of each drug within the treatment regimen are administered on the same day. Given potential different treatment schedules it is clear that during following days/weeks/months administrations may not always occur on the same day. In general, parallel administration aims for both drugs being present in the body at the same time at the beginning of each treatment cycle. Sequential administration in this context preferably means that both treatments are started sequentially, i.e. the first administration of the first drug occurs at least one day, preferably a few days or one week, earlier than the first administration of the second drug in order to allow a pharmacodynamic response of the body to the first drug before the second drug becomes active. Treatment schedules may then be overlapping or intermittent, or directly following each other.

“about”, when used together with a value, means the value plus/minus 10%, preferably 5% and especially 1% of its value.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

The term “at least one” such as in “at least one chemotherapeutic agent” may thus mean that one or more chemotherapeutic agents are meant. The term “combinations thereof” in the same context refers to a combination comprising more than one chemotherapeutic agents.

“wt” is used for wild type.

“qxw”, from Latin quaque/each, every for every x week, e.g. q2w for every second week.

“s.c.” for subcutaneously.

“i.v.” for intravenously.

“i.p.” for intraperitoneally.

“SoC” for standard of care.

Technical terms are used by their common sense. If a specific meaning is conveyed to certain terms, definitions of terms will be given in the following in the context of which the terms are used.

### DESCRIPTION OF THE INVENTION

In a first aspect, the present invention relates to an interleukin-2/interleukin-15 receptor βγ (IL-2/IL-15Rβγ) agonist for use in treating cancer in a patient, wherein said IL-2/IL-15Rβγ agonist,
a. is administered simultaneously with or sequentially to a cytotoxic compound capable of inducing immunogenic cell death (ICD),
b. is administered simultaneously with or sequentially to applying a modality capable of inducing ICD,
c. is administered simultaneously with a cytotoxic compound capable of inducing ICD and simultaneously with a modality capable of inducing ICD,
d. is administered simultaneously with a cytotoxic compound capable of inducing ICD and sequentially to a modality capable of inducing ICD,
e. is administered sequentially to a cytotoxic compound capable of inducing ICD and simultaneously with a modality capable of inducing ICD, or
f. is administered sequentially to a cytotoxic compound capable of inducing ICD and sequentially to a modality capable of inducing ICD.

Disclosed herein are combination therapies that enhance the antitumor effect of IL-2/IL-15Rβγ agonists targeting primarily the mid-affinity IL-2/IL-15Rβγ receptor and of cytotoxic compound capable of inducing ICD and/or modalities capable of inducing ICD. Such enhancement of the antitumor effect may lead to an improved efficacy of the combined treatment compared to each single treatment, as for example measurable in an increased response rates, overall survival or progression-free survival, and/or may lead to applying lower doses of/less intense treatment with the cytotoxic compounds/modalities inducing ICD—thereby reducing their toxicities/side effects—without hampering antitumor effect compared to the monotherapy. Lowering the dose of highly toxic compounds/modalities by combination with the claimed IL-2/IL-15Rβγ agonists may lead to increasing the patient population eligible for such toxic compounds/modalities, as patients in an earlier stage of a given treatment may accept such combined treatment based on a more acceptable side effect profile, or tumor indications where practitioners previously were hesitant to use a toxic compound/modality due to side effects may now, in combination with IL-2/IL-15Rβγ agonists, become treatable for such combination. More specifically, the combination of the IL-2/IL-15Rβγ agonists with the cytotoxic compound capable of inducing ICD or the modality capable of inducing ICD lead to a synergistic enhancement or the antitumor activity of the combined treatment compared to the individual treatments.

The inventors have observed in vitro that the activation of NK cells from human PBMC by a an IL-2/IL-15Rβγ agonist (here SOT101/SO-C101/RLI-15) as a measure for mounting a strong innate antitumor response was strikingly stronger, if dying tumor cells were expressing the ICD markers Hsp70, Hsp90 and CRT as well as increasing expression of NK cell ligands CD112, CD155, ULBP3 and ULBP2/5/6, here induced by incubation with trastuzumab emtansine/Kadcyla®. Trastuzumab emtansine is an antibody-drug conjugate consisting of the humanized monoclonal antibody trastuzumab/Herceptin® directed to the tumor target HER2 covalently linked to the cytotoxic compound mertansine/DM1. Due to the fact that Kadcyla had been washed away prior to the incubation with the activated PBMC, a direct interaction of Kadcyla with the immune cells can be excluded and the inventors conclude that the early apoptotic state/ICD of the cell population largely contributes to this effect, and therefore other cytotoxic compounds capable of inducing ICD or modalities capable of inducing ICD will have a very similar synergistic effect.

Similarly, the combination of SOT102, a CLDN18.2-targeted ADC with the anthracycline PNU-159682 as a toxin, synergized with SOT101 in an NK-cell based cytotoxicity assay in vitro, such effect being caused or contributed by the induction of ICD. ADCs with PNU as the toxin have been previously described to induce ICD (D'Amico, Menzel et al. 2019). The activation of danger signals by toxins/chemotherapies but also radiotherapy were reported to produce for example an augmentation of Hsp70 cell-surface expression on tumor cells promoting NK cell mediated cytotoxicity in vitro and in vivo (Zingoni, Fionda et al. 2017). Recently, the externalized CRT, a hallmark of ICD, has been identified as the activating ligand of the NKp46 receptor of NK cells, and its binding triggers NKp46 signaling, whereas inhibition of this interaction inhibits NKp46-mediated killing (Santara, Crespo et al. 2021).

Accordingly, the inventors conclude that there is a direct mechanistical link between the induction of ICD of tumor cells by cytotoxic compounds and/or modalities inducing ICD as described herein, making them more susceptible to the cytotoxic activity of NK cells, which in turn can be potentiated by the described IL-2/IL-15Rβγ agonists, e.g., SOT101, which are potent activators of NK cells.

NK cell activation is considered to have predictive value of an in vivo antitumor efficacy, and indeed a similar synergistic effect was observed in a murine orthotopic breast cancer model in vivo.

Generally, the observed synergistic effect of the combination of the ICD inducing cytotoxic compound or modality with the IL-2/IL-15Rβγ agonist may be used to (i) reduce the dosage of the cytotoxic compound or intensity of the modality (e.g., non-ablative/low-dose radiation therapy) in order to reduce side effects induced by the cytotoxic compound or modality resulting—due to the combined action—at least at the same treatment benefit for the patient, (ii) avoid relapses of the tumor disease due to the strong ICD-induced immune surveillance in the combination treatment, and/or (iii)—in case of antibody-drug-conjugates—broaden the patient population as also patients having a lower (compared to the target level of the label of the respective ADC) target expression would benefit from the combined treatment.

In one embodiment the IL-2/IL-15Rβγ agonist is administered sequentially prior to and/or subsequent to said cytotoxic compound capable of inducing ICD, or prior to and/or subsequent to said modality capable of inducing ICD. Given the different dosing/treatment schedules of such cytotoxic compounds and IL-2/IL-15Rβγ agonists or such treatment modalities and IL-2/IL-15Rβγ agonists it is quite typical that the IL-2/IL-15Rβγ agonists are not administered at the very same moment as such cytotoxic compounds or modalities.

In a preferred embodiment in the case of sequential administration, the IL-2/IL-15Rβγ agonist is administered subsequently to said cytotoxic compound capable of ICD or subsequent to said modality capable of inducing ICD. As the induction of ICD by such cytotoxic compound or such modality takes some time, it is believed to be beneficial that the IL-2/IL-15Rβγ agonist is administered subsequently, so that sufficient time is provided that the changes to the cell surface as well as the release of the soluble mediators of ICD has taken place before the NK and CD8 cells are being activated to boost the immune system against such tumor cells undergoing ICD. Preferably, the time difference between the last administration/treatment of the ICD inducing cytotoxic compound or modality and the administration of the IL-2/IL-15Rβγ agonist is between about 6 hours and about 2 weeks, more preferably between about 1 day and about 7 days, especially between about 1 day and about 4 days. The timing may differ depending on the nature of the cytotoxic compound. A free drug may induce ICD quicker than for example an ADC, due to the required processing including relatively long in vivo half-life, surface binding, internalization, trafficking through the endosomal/lysosomal pathway, construct degradation, release of the cytotoxic payload from the lysosome, and activation of cell death pathways, which may further vary from cell type and by target antigen (Bauzon, Drake et al. 2019).

In another embodiment the IL-2/IL-15Rβγ agonist and said cytotoxic compound capable of inducing ICD are provided as components of the same pharmaceutical compositions or as components of separate pharmaceutical compositions are administered simultaneously. In order to minimize the efforts for the patient to go the hospital or the medical doctor for the administration of the drugs, simultaneous treatment is preferred. It may further be feasible in certain combinations that the IL-2/IL-15Rβγ agonist and such cytotoxic compound can be co-formulated as a single pharmaceutical composition in order to simply the administration.

In one embodiment, the cytotoxic compound capable of inducing ICD is selected from the group consisting of an anthracycline; a microtubule-destabilizing agent including a vinca alkaloid, a taxane, an epothilone, eribulin, an auristatin (e.g. MMAE or MMAF), maytansine or a maytansinoid and tubulysin; bleomycin; a proteasomal inhibitor including bortezomib; topoisomerase I inhibitors including topotecan, exatecan and exatecan derivatives such as DS-8201a, DX-8951/DXd (Kitai, Kawasaki et al. 2017, Iwata, Ishii et al. 2018, Haratani, Yonesaka et al. 2020); an alkylating agent including cyclophosphamide, a platinum complex including oxaliplatin, and a pyrrolo-benzodiazepines (PBD) (Rios-Doria, Harper et al. 2017); and nucleoside analogs including gemcitabine (preferably in combination with inhibitory damage-associated molecular patterns (DAMP) blockade) (Hayashi, Nikolos et al. 2021). Cytotoxic compounds capable of inducing ICD have been repeatedly reviewed (Pol, Vacchelli et al. 2015, Diederich 2019, Zhou, Wang et al. 2019). SN38 is preferably excluded as other topoisomerase I inhibitors such as DS-8201a have higher potency and induce more immunogenic cell death (Iwata, Ishii et al. 2018).

Anthracyclines (and derivatives) are a class of cytotoxic compounds of bacterial origin applied in many tumor indications including leukemias, lymphomas, breast cancer, gastric cancer, ovarian cancer, bladder cancer and lung cancer and act mainly by intercalating into the DNA and thereby interfering with the DNA replication and transcription, e.g. by interfering with the topoisomerase II. Members of this class are daunorubicin, doxorubicin, epirubicin, idarubicin, valrubicine, nemorubicin and PNU-159682 ((3′-deamino-3″,4′-anhydro-[2″(S)-methox-y-3″(R)-oxy-4″-morpholinyl]), (briefly “PNU”)—a metabolite of nemorubicin (Quintieri, Geroni et al. 2005)—and have been proven to induce ICD (Fucikova, Kralikova et al. 2011).

Microtubule-destabilizing agents (“MDAs”) is another class of compounds which induce ICD (Diederich 2019), which is a diverse class of compounds grouped together due to their mode of action with microtubules as the target thereby impacting proliferation, trafficking, signaling and migration of cells (Dumontet and Jordan 2010). This class includes vinca alkaloids (vinblastin, vincristine, vinflunine, cevipabulin), taxanes (paclitaxel, docetaxel and others, see Table 2 of (Dumontet and Jordan 2010), whereas docetaxel is also reported to be negative for ICD despite induction of calreticulin), eribulin, epothilones including epothilone A to F, 7A7 and patupilone, auristatins including monomethyl auristatin E (MMAE) and monomethyl auristatin F (MMAF), maytansine and maytansinoids such as mertansine/emtansine (DM1), ansamitocin and ravtansine/soravtansine (DM4), tubulysin, colchicine and others (see e.g. Fig. 1 of Dumontet and Jordan (2010)), reviewed by Diederich (2019), Dudek et al. (2013), Dumontet and Jordan (2010) and Gerber et al. (2016).

Further cytotoxic compounds inducing ICD are bleomycin; proteasomal inhibitors like bortezomib and Shikonin; alkylating agents like cyclophosphamide, mitoxantrone, platinum complexes including oxaliplatin, cardiac glycosides (Dudek, Garg et al. 2013, Pol, Vacchelli et al. 2015, Gerber, Sapra et al. 2016), and pyrrolo-benzodiazepines (PBD) (Zhou, Wang et al. 2019), preferably its prodrug pro-PBD (Vlahov, Qi et al. 2017). Shikonin, a bioactive phytochemical inhibiting the 20S subunit of the proteasome (being a proteasome inhibitor like bortezomib), has been shown to induce ICD in cancer cells, characterized by induction of expression of HSP70, calreticulin and GRP78, and induce functional maturation of DCs. Further, calicheamicins, a class of enediyne antitumor antibiotics derived from Micromonospora echinospora, have been reported to induce immunogenic cell death (Tan, Lam et al. 2018). Calicheamicin γ₁^{I} (LL-E33288) is the most renown member, further calicheamicin derivatives are described in WO 2019/110725.

Topotecan and DX-8951/DXd also has been described to induce immunogenic cell death (Kitai, Kawasaki et al. 2017, Iwata, Ishii et al. 2018, Haratani, Yonesaka et al. 2020), as it a upregulated the expression of DC maturation and activation markers both in vitro and in vivo and increased the intratumoral DC population in vivo (Iwata, Ishii et al. 2018) and observed release of HMGB-1 from DXd-treated cancer cells (Haratani, Yonesaka et al. 2020).

Several of the ICD inducing cytotoxic compounds are highly interesting as payloads for ADCs, including anthracyclines (Minotti, Menna et al. 2004), (WO2016/102679A1), MMAE, DM1 (Diederich 2019), PBD (Rios-Doria, Harper et al. 2017, Zhou, Wang et al. 2019) and tubulysin (Rios-Doria, Harper et al. 2017). Accordingly, it is a preferred embodiment of the present invention that such cytotoxic compound capable of inducing ICD is covalently linked to an antibody forming an antibody-drug conjugate (ADC). ADCs is a rapidly growing class of anticancer drugs which targets the cytotoxic compound to a molecular target typically expressed on the surface of a target cell by chemical linkage to an anti-cancer antibody thereby reducing systemic exposure and toxicity. Various design strategies are presently employed including target selection, design of the antibody moiety, the covalent linker between antibody and the cytotoxic compound, and the selection of the cytotoxic compound or, in this context often referred to, the payload. Currently, four ADC products are marketed (gemtuzumab ozogamicin/Mylotarg®, brentuximab vedotin/Adcetris®, trastuzumab emtansine/Kadcyla® and inotuzumab ozogamicin/Besponsa®) and more than 60 ADCs are presently clinically developed (Khongorzul, Ling et al. 2020), with 3 additional approvals in 2019 (Trastuzumab deruxtecan/Enhertu®, enfortumab vedotin/Padcev® and polatuzumab vedotin (Polivy®). In 2020, Sacituzumab govitecan (Trodelvy®) and Belantamab mafodotin-blmf (Blenrep®) were approved by FDA, followed by Loncastuximab tesirine-lpvl (Zynlonta®) and Tisotumab vedotin-tftv (Tivdak®) in 2021.

In a preferred embodiment, the cytotoxic compound capable of inducing ICD is an anthracycline, a maytansine or maytansinoid, a topoisomerase I inhibitor or a calicheamicin derivative. Specifically, ADCs with anthracyclines as payloads and ADCs with maytansine or maytansinoids have been described to induce ICD. D'Amico et al. (2019) describe that an ADC composed of trastuzumab linked to PNU (an anthracycline, T-PNU) lead to ICD in a human HER2-expressing syngeneic breast cancer model resistant to trastuzumab and ado-trastuzumab emtansine in a CD8⁺ T cell dependent manner, thus confirming the PNU mediated anti-tumor immune response also in the context of an ADC. Further, the T-PNU promoted the generation of immunological memory protecting the treated animals from tumor re-challenge. Bauzon et al. (2019) showed that maytansine and maytansine-based ADCs induced three major hallmarks of ICD in vitro and conclude that maytansine, MMAE, tubulysin and PBD appear to have a similar immunostimulatory activity in vivo. Accordingly, brentuximab vedotin, an MMAE-conjugated anti-CD30 antibody, increased the number of tumor-infiltrating CD8⁺ T cells and efficacy was proven in patients expressing little or no target antigen suggesting an indirect, potentially immune mediated mechanism (summarized in Bauzon, Drake et al. 2019).

Preferably, said anthracycline is selected from the group consisting of daunorubicin, doxorubicin, epirubicin, idarubicin, mitoxantrone and PNU-159682 (PNU), and said maytansine or maytansinoid is selected from maytansine, mertansine/emtansine (DM1), ansamitocin and ravtansine/soravtansine (DM4).

In another preferred embodiment, the cytotoxic compound capable of inducing ICD is a topoisomerase I inhibitor, preferably topotecan, exatecan and exatecan derivatives such as DX-8951/DXd. Both trastuzumab deruxtecan (DS-8201a) with the anti-HER2 antibody trastuzumab coupled to the exatecan derivative DX-8951/DXd and patritumab deruxtecan (U3-1402) with the anti-HER3 antibody patritumab coupled to DX-8951/DXd are approved/clinical stage ADCs with a topoisomerase I payload shown to induce immunogenic cell death.

In another preferred embodiment, the cytotoxic compound capable of inducing ICD is a calicheamicin derivative, preferably Calicheamicin γ₁^{I} (LL-E33288) or calicheamicin derivatives described in WO 2019/110725.

In a further embodiment, said antibody is an antibody which specifically binds to HER2, preferably trastuzumab, SYD985 or MEDI4276, more preferably trastuzumab; binds to Nectin-4, preferably enfortumab; binds to CD33, preferably gemtuzumab or IMGN779, more preferably gemtuzumab; binds to CD30, preferably brentuximab; binds to CD22, preferably inotuzumab, or CD79B, preferably polatuzumab. Further preferred targets/antibodies are TROP2/sacizuzumab, FOLR1/mirvetuximab, BCMA/GSK2857916, GPNMB/glembatumumab, Mesothelin/anetumab, CEACAM5/labetuzumab or SAR408701, PSMA/antibody of NCT01695044 and NCT02020135 or MEDI3726, CD19/coltuximab, EGFR/depatuxizumab, ENPP3/AGS-16C3F, EFNA4/PF-06647263, HER3/patritumab, CD352A/SGN-CD352A, CD37/AGS67E, FLT3/AGS-62P1, ROR-1/NBE-002 and Claudin18.2/zolbetuximab or a humanized variant thereof (e.g. disclosed in WO2021/111003A1) or humanized antibodies, especially hCl1a, disclosed in table 3 of WO2021/130291A1.

In another preferred embodiment, said ADC is trastuzumab emtansine/Kadcyla®, trastuzumab deruxtecan/Enhertu®, gemtuzumab ozogamicin/Mylotarg®, inotuzumab ozogamicin/Besponsa®, brentuximab vedotin/Adcetris®, enfortumab vedotin/Padcev® and polatuzumab vedotin/Polivy®. Especially preferred is trastuzumab emtansine (also referred to as ado-trastuzumab emtansine). Further especially preferred is enfortumab vedotin. A further preferred ADC is Sacituzumab govitecan. A further preferred ADC is Belantamab mafodotin-blmf. A further preferred ADC is Loncastuximab tesirine-lpyl. A further preferred ADC is Tisotumab vedotin-tftv.

In another preferred embodiment, the IL-2/IL-15Rβγ agonist is for use in a patient suffering from tumors expressing HER2, preferably wherein the patient has been diagnosed with having a tumor with low to intermediate HER2 expression. The inventors have shown synergy with trastuzumab emtansine (Kadcyla®), which is approved for the treatment of patients with HER2-positive tumors, specifically HER2-positive metastatic breast cancer who previously received trastuzumab and a taxane separately or in combination. HER2-positive according to the Kadcyla® label means patients with breast cancer having HER2 overexpression defined as 3+ IHC by Dako HercepTest™ or defined as FISH amplification ratio ≥2.0 by Dako HER2 FISH PharmDx™ test kit, accordingly a high expression of HER2. In one embodiment, selection of HER2 patients would be done according to the label of trastuzumab emtansine, i.e., patients would be selected for having high HER2 expression, e.g. being HercepTest™ 3+. For the combination treatment with the IL-2/IL-15Rβγ agonist in patients with a high HER2 expression the inventors expect that the typically observed high rate of relapses for the Kadcyla treatment is markedly reduced, as it was observed in the orthotopic huHER2/EMT-6 breast cancer model (see Example). Alternatively or additionally, one may for such combination treatment reduce the dose of the Kadcyla® (or the ADC in general), in order to reach in the combination with the IL-2/IL-15Rβγ agonist at least the same efficacy but at reduced side effects.

In another embodiment, also low to intermediate HER2-expressing patients are selected for the combination therapy of Kadcyla® with the IL-2/IL-15Rβγ agonist, preferably SOT101. Given the synergistic enhancement of the treatment compared to the single treatments the inventors reason that a lower expression would be sufficient to obtain a treatment benefit for the patients.

Other HER2 over-expressing tumors are ovarian, stomach, adenocarcinoma of the lung, uterine cancer (e.g., uterine serous endometrial carcinoma), salivary duct carcinoma, renal, endometrial, colorectal, head and neck, urothelial, breast and cervical carcinoma, which makes them together with breast cancer preferred tumor indications for the treatment with the IL-2/IL-15Rβγ agonist in combination with trastuzumab emtansine, preferably with confirmed status of HER2 overexpression.

In another preferred embodiment, the IL-2/IL-15Rβγ agonist is for use in a patient suffering from tumors expressing Nectin-4, preferably wherein the patient has been diagnosed with having a locally advanced or metastatic urothelial cancer who have previously received a programmed death receptor-1 (PD-1) or programmed death-ligand 1 (PD-L1) inhibitor, and a platinum-containing chemotherapy in the neoadjuvant/adjuvant, locally advanced or metastatic setting. Enfortumab vedotin (also referred to as enfortumab vedotin-ejfv) has been approved for this indication and given the known induction of ICD by its MMAE payload, synergy with IL-2/IL-15βγ receptors is expected by the inventors based on the findings of the invention. Nectin-4 is an adhesion protein located on the surface of cells and was detected in all patients tested in the clinical trial leading to approval. Accordingly, no test for patient stratification is required. Administration of enfortumab vedotin would preferably be pursued according to its label. Other Nectin-4 positive tumors are bladder cancer in general, ovarian cancer, lung cancer, prostate cancer, esophageal cancer, breast cancer, pancreatic cancer, head and neck cancer, cervical cancer, which makes the together with urothelial cancer preferred indications for the treatment with the IL-2/IL-15Rβγ agonist in combination with enfortumab vedotin. Similarly, recently approved Tisotumab vedotin-tftv is using MMAE as a payload, here targeted to Tissue factor for the indication cervical cancer. Accordingly, the combination of Tisotumab vedotin-tftv with an IL-2/IL-15Rβγ agonist, preferably SOT101, is a further embodiment of the invention.

In another preferred embodiment, the IL-2/IL-15Rβγ agonist is for use in a patient suffering from tumors expressing CLDN18.2 (or Claudin 18.2), preferably wherein the patient has been diagnosed with having gastric or pancreatic cancer, for example by using the antibody Zolbetuximab (IMAB362) disclosed in WO 2007/059997 and WO 2016/165762. WO 2016/166122 discloses anti-CLDN18.2 monoclonal antibodies that can be efficiently internalized upon CLDN18.2 binding and therefore, are suitable for ADC development. Other antibodies suitable for ADC development are human variants of Zolbetuximab, e.g., disclosed in WO2021/111003A1, or humanized antibodies, especially hCl1a, disclosed in table 3 of WO2021/130291A1. Suitable ADCs targeting CLDN18.2 are described in WO2022/136642A1, including SOT102 as described in example 7 therein, making especially the combination of SOT102 with an IL-2/IL-15Rβγ agonist, preferably SOT101, another embodiment of the invention.

In another embodiment the modality capable of inducing ICD is selected from high hydrostatic pressure (HHP), photodynamic therapy, UV radiation, radiotherapy, gamma radiation and thermotherapy. HHP refers to the treatment of tumor cells with high hydrostatic pressure as described in for example by WO 2013/004708, WO 2015/097037, WO 2019/145469, WO 2019/145471, Fucikova et al. (2014), Obeid et al. (2007) and Adkins et al. (2018). In one embodiment, such HHP modality is a dendritic cell vaccine, wherein whole tumor cells were driven into ICD by high hydrostatic pressure (HHP) as described in WO 2013/004708 and WO 2015/097037 (see for example examples 1 to 4 of WO 2013/004708 and examples 2 and 3 of WO 2015/097037). In brief, whole tumor cells from cell lines or from the patient are treated by HHP between 200 and 300 MPa for 10 min to 2 hours. Such a treatment will induce ICD in the treated tumor cells which may be characterized by expression of immunogenic molecules on the cell surface such as HSP70, HSP90 and calreticulin and the release of late apoptotic markers HMGB1 and ATP and thus increase the uptake of these cells by dendritic cells (DC), resulting in loaded DCs presenting multiple tumor antigens. Prior to being loaded on DCs, the apoptotic tumor cells may be cryopreserved. The whole tumor cells loaded upon the DC vaccine are preferably allogeneic to the patient, e.g., tumor cell lines, which have an overlap of expressed tumor antigens with the typical tumor antigens of the tumor disease to be treated. Whereas autologous tumor cells purportedly have a better match with the patient's tumor antigens, in practice it is highly complicated to manufacture a DC vaccine from autologous tumor biopsies. In turn, the DCs may be derived from monocytes that are autologous to the patient being treated. As used herein, the term “monocytes” refers to leukocytes circulating in the blood characterized by a bean-shaped nucleus and by the absence of granules. Monocytes can give rise to dendritic cells. The monocytes can be isolated from a patient's blood by any technique known to one of skill in the art, the preferred method being leukapheresis. Leukapheresis allows to collect monocytes that are autologous to the patient being treated, to be used for the preparation of the DC vaccine. Leukapheresis may be performed by any technique known to one of skill in the art.

Other treatment modalities inducing ICDs have been described in the art and include photodynamic therapy, preferably with hypericin; UV radiation, preferably UVC radiation; radiotherapy including brachytherapy; oncolytic virus therapy; and thermotherapy, all of which have also been described to induce ICD (Dudek, Garg et al. 2013, Adkins, Sadilkova et al. 2017, Zhou, Wang et al. 2019) and therefore are preferred modalities to induce ICD. Briefly summarized, short-wavelength ultraviolet radiation (UVC) has been described to induce an inflammatory response in the skin and can induce ICD determinants such as calreticulin, HMGB1 and HSP70.

Similarly, radiotherapy can induce besides direct cell killing the so-called “abscopal effect”, i.e. the T-cell mediated growth delay of tumors located far from the irradiated area, which is explained by radiotherapy's ability to reproducibly induce ICD again characterized by exposure of calreticulin, HSP70 and release of HMGB1. The exposure/release of DAMPs from the irradiated cells are believed to stimulate DCs in vivo (similar to the above described DC vaccination with tumor cells undergoing ICD ex vivo). Preferably, local high-dose radiotherapy is applied to induce ICD as it has been shown to increase the number of tumor-infiltrating active DCs. Also, lower dose, non-ablative or sub-ablative radiotherapy may have advantages for the claimed combinations, as it has been described to also reprogram macrophages towards the beneficial M1 phenotype. Suitable doses and fractionation of radiotherapy is summarized in Golden and Apetoh (2015).

Also photodynamic therapy based on the photosensitizer hypericin was shown to induce ICD in cancer cells and established on the level of phagocytosis and maturation a highly productive interface with DCs again by inducing the immunological signatures of ICD in cancer cells, e.g. calreticulin, HSP70 and others. Nano pulse stimulation with ultrashort electrical pulses in the nanosecond range has been described to induce ICD as well, like the treatment with oncolytic viruses, which trigger during oncolytic virus-mediated oncolysis of cancer cells the calreticulin surface exposure, ATP release and ER stress, again hallmarks of ICD. Further specific treatments described to be capable of inducing ICD are near-infrared photoimmunotherapy, oxygen-boosted photodynamic therapy, nanosized drug carriers or thermotherapy (Dudek, Garg et al. 2013, Adkins, Sadilkova et al. 2017, Zhou, Wang et al. 2019). Accordingly, there is a growing field of treatment modalities unified by the specific features of inducing ICD in tumor cells and thereby triggering a specific anti-immune response likely mediated by DCs. Accordingly, all these treatment modalities are preferred for combination with the treatment with IL-2/IL-15Rβγ agonists.

In one embodiment the IL-2/IL-15Rβγ agonist is an IL-15/IL-15Rα complex. Whereas IL-2 and IL-15 share the β and the γ receptor and accordingly have an overlapping downstream intracellular signaling, wtIL-2 complexes bear the disadvantage that they activate the IL-2Rαβγ expressed on T_{regs} and lung endothelium, which should be avoided. Different strategies are employed to modify IL-2 to avoid the binding to the IL-2α receptor using IL-2 muteins and/or chemical modifications, which all have certain disadvantages, e.g., reducing the activity (e.g. IL2v, NKTR-255), complicated expression systems (THOR-707) or expensive chemical modifications (e.g. PEGylated complexes). In turn, IL-15 as such still binds to the IL-15Rαβγ again on T_{regs}. Therefore, complexes comprising IL-15 or an IL-15Rα derivative, who simulate the trans-presentation of the IL-15Rα and therefore limit the binding to the IL-2/IL-15βγ receptor are preferred.

Preferably, the IL-15 is the mature wtIL-15 having the sequence of SEQ ID NO: 4. Further, many activating or inactivating mutations have been described in the art in order to achieve various defined changes to the molecule: D8N, D8A, D61A, N65D, N65A, Q108R for reducing binding to the IL-15Rβγβγ_{c} receptors (WO 2008/143794A1); N72D as an activating mutation (in ALT-803); N1D, N4D, D8N, D30N, D61N, E64Q, N65D, and Q108E to reduce the proliferative activity (US 2018/0118805); L44D, E46K, L47D, V49D, I50D, L66D, L66E, I67D, and 167E for reducing binding to the IL-15Rα (WO 2016/142314A1); N65K and L69R for abrogating the binding of IL-15Rβ (WO 2014/207173A1); Q101D and Q108D for inhibiting the function of IL-15 (WO 2006/020849A2); S7Y, S7A, K10A, K11A for reducing IL-15Rβ binding (Ring, Lin et al. 2012); L45, S51, L52 substituted by D, E, K or R and E64, I68, L69 and N65 replaced by D, E, R or K for increasing the binding to the IL-15Rα (WO 2005/085282A1); N71 is replaced by S, A or N, N72 by S, A or N, N77 by Q, S, K, A or E and N78 by S, A or G for reducing deamidation (WO 2009/135031A1); WO 2016/060996A2 defines specific regions of IL-15 as being suitable for substitutions (see para. 0020, 0035, 00120 and 00130) and specifically provides guidance how to identify potential substitutions for providing an anchor for a PEG or other modifications (see para. 0021); Q108D with increased affinity for CD122 and impaired recruitment of CD132 for inhibiting IL-2 and IL-15 effector functions and N65K for abrogating CD122 affinity (WO 2017/046200A1); N1D, N4D, D8N, D30N, D61N, E64Q, N65D, and Q108E for gradually reducing the activity of the respective IL-15/IL-15Rα complex regarding activating of NK cells and CD8 T cells (see FIG. 51, WO 2018/071918A1, WO 2018/071919A1). Mutating K86 (e.g. K86R) has been described to increase the stability, as K86 is a putative site for ubiquitin-dependent degradation and N112 (e.g. N112) has been described to enhance IL-15 activity (see WO 2018/151868). The substitution of L52C has been made to introduce an additional cysteine for a disulfide bond with a mutated IL-15Rα sushi domain (Hu, Ye et al. 2018). Additionally or alternatively, the artisan can easily make conservative amino acid substitutions. Given the high potency of the IL-15 molecule, activity reduction by a factor of even thousand-fold may still be compensated by higher dosing or by the pharmacodynamic effect of molecules with a longer half-life due to a markedly increased molecular weight. Increased molecular weight can be achieved for example by fusion or covalent linkage of the IL-15/IL-15Rα complex to an Fc part of an antibody, to an antibody, to serum albumin or by PEGylation.

Therefore, multiple mutations may easily be combined in a protein without harming its biological/commercial value. Therefore, preferably, the IL-15 derivative has at least 0.1% of the activity of human IL-15, preferably 1%, more preferably at least 10%, more preferably at least 25%, even more preferably at least 50%, and most preferably at least 80%. In one embodiment, the activity is measured as the effect of IL-15 on the proliferation induction of the kit225 cell line (HORI et al., Blood, vol. 70 (4), p: 1069-72, 1987).

Still, it is preferred to limit the numbers of mutations/substitutions as every additional mutation at least theoretically increases the risk of inducing immunogenicity and thereby the potential of generating anti-drug antibodies in the patient, which may limit the activity of the complex with increasing numbers of administrations. Therefore, preferably, the IL-15 derivative has a percentage of identity of at least 92%, preferably of at least 96%, more preferably of at least 98%, and most preferably of at least 99% with the amino acid sequence of the mature human IL-15 (114 aa) (SEQ ID NO: 4).

Still, also for IL-15, chemical modification as known in the art, e.g. by PEGylation or other posttranslational modifications (see WO 2016/060996A2, WO 2017/112528A2, WO 2009/135031A1) and may be preferably employed for the IL-15/IL-15Rα complex of the invention.

IL-15Rα in the IL-15/IL-15Rα complex refers to an IL-15Rα derivative, which preferably comprises at least the sushi domain of wt IL-15Rα, but does not comprise the transmembrane and the intracellular domains of wt IL-15Rα. Further, it preferably does not comprise the 30 aa peptide leader sequence, which is typically cleaved off during expression. The IL-15Rα sushi domain (or IL-15Rαₛᵤₛₕᵢ, SEQ ID NO: 6) is the domain of IL-15Rα which is essential for binding to IL-15 (Wei, Orchardson et al. 2001) and therefore is the minimum fragment of IL-15Rα in the IL-15/IL-15Rα complex. The sushi+ fragment (SEQ ID NO: 7) comprising the sushi domain and part of hinge region, defined as the fourteen amino acids which are located after the sushi domain of this IL-15Rα, in a C-terminal position relative to said sushi domain, i.e., said IL-15Rα hinge region begins at the first amino acid after said (C4) cysteine residue, and ends at the fourteenth amino acid (counting in the standard “from N-terminal to C-terminal” orientation). The sushi+ fragment reconstitutes full binding activity to IL-15 (WO 2007/046006) and accordingly is a preferred. Accordingly, preferred IL-15Rα derivatives comprise at least the sushi domain (aa 33-93) but do not extend beyond the extracellular part of the mature IL-15Rα being amino acids 31-209 of SEQ ID NO: 5. Specifically preferred IL-15Rα derivatives are the sushi domain of IL-15Rα (SEQ ID NO: 6) and the sushi+ domain of IL-15Rα (SEQ ID NO: 7). The IL-15Rα sushi+ can further be C-terminally extended in order to enlarge the molecule and thereby increase its serum half-life, as done for hetIL-15. Accordingly, other preferred IL-15Rα derivatives are the soluble forms of IL-15Rα (from amino acids 31 to either of amino acids 172, 197, 198, 199, 200, 201, 202, 203, 204 or 205 of SEQ ID NO: 5, see WO 2014/066527, (Giron-Michel, Giuliani et al. 2005)).

In another embodiment the IL-15Rα derivative may include natural occurring or introduced mutations. Natural variants and alternative sequences are e.g. described in the UniProtKB entry Q13261 (https://www.uniprot.org/uniprot/Q13261). Further, the artisan can easily identify less conserved amino acids between mammalian IL-15Rα homologs or even primate IL-15Rα homologs in order to generate derivatives which are still functional. The IL-15Rα derivative functions due to its binding to IL-15 and thereby forming a complex that mimics trans-presentation of IL-15 in the immunological synapse by an antigen-presenting (e.g. dendritic) cell to an immune effector cell (e.g. NK or CD8⁺ T-cell). Further, due to its presence it blocks binding to the IL-15αβγ receptor. It is clear to the artisan that especially in a co-valent fusion protein comprising both the IL-15 (or a derivative thereof) and the IL-15Rα derivative, the binding of the IL-15Rα derivative to the IL-15 (or its derivative) can be markedly reduced without losing its activity as the co-valent linkage compensates for the reduced bind and the molecules would still from a stable complex. Further, the substitution S40C of IL-15Rα has been made to introduce an additional cysteine for forming a disulfide bond with a mutated IL-15 (Hu, Ye et al. 2018).

Respective sequences of mammalian IL-15Rα homologs are described in WO 2007/046006, page 18 and 19. Again, the number of mutation compared to the wt sequence should be limited to avoid increased immunogenicity, so the IL-15Rα derivative preferably comprising an amino acid sequence having a percentage of identity of at least 92%, preferably of at least 96%, more preferably of at least 98%, and even more preferably of at least 99%, and most preferably 100% identical with the respective wt sequence of the same length, more preferably with the amino acid sequence of the sushi domain of human IL-15Rα (SEQ ID NO: 6) within the overlapping sequence and, especially with the amino acid sequence of the sushi+ domain of human IL-15Rα (SEQ ID NO: 7) within the overlapping sequence.

Preferably, an IL-15Rα derivative has at least 10% of the binding activity of the human sushi domain to human IL-15, e.g. as determined in (Wei, Orchardson et al. 2001), more preferably at least 25%, even more preferably at least 50%, and most preferably at least 80%.

In one embodiment, the IL-2/IL-15Rβγ agonist is an interleukin 15 (IL-15)/interleukin-15 receptor alpha (IL-15Rα) complex, wherein the complex is a fusion protein comprising the sushi domain of human IL-15Rα or a derivative thereof, a flexible linker and the human IL-15 or a derivative thereof, preferably wherein the human IL-15Rα sushi domain comprises the amino acid sequence of SEQ ID NO: 6, and wherein the human IL-15 comprises the amino acid sequence of SEQ ID NO: 4. Such fusion protein is preferably in the order (from N- to C-terminus) IL-15_Ra-linker-IL-15 (RLI-15). Other examples of fusion proteins are described in WO 2018/071919A1, where the sushi domain of IL-15Rα is fused through a disulfide bond to IL-15 (e.g. XENP22004), through covalent linkage to a heterodimeric Fc (e.g. XENP22013, XENP22357, XENP22639, or with two IL-15Rα(sushi)/IL-15 fusion: e.g. XENP22634). Also WO 2015/103928 discloses alternative formats to build IL-15/IL-15Rα complexes e.g. by forming stable complexes through the interaction of a first and a second Fc variant where one is linked to the IL-15 (or derivative thereof) and the other one to the IL-15Rα derivative. Hu et al. (Hu, Ye et al. 2018) describes the IL-15/IL-15Rα complex P22339, where the IL-15 is covalently linked to the sushi domain of IL-15Rα by introducing a novel disulfide bond between L52C of IL-15 and S40C of IL-15Rα.

An especially preferred IL-2/IL-15Rβγ agonist is the fusion protein designated RLI2 having the sequence of SEQ ID NO: 9. RLI2 (also known as SO-C101 or CYT101) is subject of the clinical trial NCT04234113 and therefore is especially suitable for the development in combination with the ICD inducing cytotoxic compounds and/or the ICD inducing modalities.

In a preferred embodiment the IL-15/IL-15Rα complex is a fusion protein comprising the amino acid sequence of SEQ ID NO: 9, especially consisting of the amino acid sequence of SEQ ID NO: 9, and the ADC comprises an antibody which specifically binds to HER2, preferably wherein the antibody is trastuzumab. As the inventors have shown, SOT101/RLI2 together with trastuzumab emtansine have shown to act synergistically both in vitro and in vivo and therefore make this combination especially preferred. Another preferred combination is SOT101/RLI2 together with SOT102 as described herein. In one embodiment, SOT101 is especially preferred as it provides a number of advantages over other IL-2/IL-15Rβγ agonists. It binds with high affinity to the mid-affinity receptor composed of the β and γ chains, whereas IL-2- and IL-15 based molecules with steric or mutational hinderance of α-chain binding bind with a lower affinity to the mid-affinity receptor. However, trans-presentation of IL-15 with the membrane bound IL-15Rα or soluble IL-15/IL-15Rα complexes are believed to, through stronger and more persistent signaling, result in metabolically more active, larger in size and more proliferative T cells compared to cells stimulated by soluble IL-15 as such, i.e., binding with mid affinity to the mid-affinity receptor leading to more potent phenotypic response (Arneja, Johnson et al. 2014). Further, SOT101 is a fusion protein, which avoids dissociation of non-covalent IL-15/IL-15Rα complexes such as hetIL-15, ALT803 or other IL-15Ra/Fc-fusion non-covalently binding IL-15, which in respective dilution in the blood may dissociate and therefore both lose their specificity and high affinity. And, SOT101 with its relatively short in vivo half-life is a very potent stimulator of NK cells even at low doses (Antosova, Podzimkova et al. 2020), which the inventors have shown to synergize with ICD induction in a model fully dependent of NK cells (in absence of T cells, Example). Accordingly, in one embodiment, the present invention provides SOT101 in combination with a cytotoxic compound capable of inducing ICD or a modality capable of inducing ICD.

Preferably, the IL-2/IL-15Rβγ agonist is administered subcutaneously (s.c.) or intraperitoneally (i.p.), whereas s.c. is even more preferred. The cytotoxic compounds inducing ICD are preferably administered according to their approved label e.g. as approved by the FDA, typically intravenously (i.v.).

In a preferred embodiment, the IL-2/IL-15Rβγ agonist is further combined with an immune checkpoint inhibitor (or in short checkpoint inhibitor). Check point inhibitors or more precisely immune check point inhibitors, refers to a type of drug that blocks certain proteins made by some types of immune system cells, such as T cells, and some cancer cells. These proteins help keeping immune responses in check and can keep T cells from killing cancer cells. When these proteins are blocked, the “brakes” on the immune system are released and T cells are able to kill cancer cells better. Checkpoint inhibitors are accordingly antagonists of immune inhibitory checkpoint molecules or antagonists of agonistic ligands of inhibitory checkpoint molecules. Examples of checkpoint proteins found on T cells or cancer cells include PD-1/PD-L1 and CTLA-4/B7-1/B7-2 (definition of the National Cancer Institute at the National Institute of Health, see hits://www.cancer.gov/publications/dictionaries/cancer-terms/def/immune-checkpoint-inhibitor), as for example reviewed by Darvin et al. (2018). Examples of such check point inhibitors are anti-PD-L1 antibodies, anti-PD-1 antibodies, anti-CTLA-4 antibodies, but also antibodies against LAG-3 or TIM-3, or blocker of BTLA currently being tested in the clinic (De Sousa Linhares, Leitner et al. 2018). Further promising check point inhibitors are anti-TIGIT antibodies (Solomon and Garrido-Laguna 2018). Examples of anti-PD-L1 antibodies are avelumab, atezolizumab, durvalumab, KN035, MGD013 (bispecific for PD-1 and LAG-3), examples of anti-PD-1 antibodies are pembrolizumab, nivolumab, cemiplimab (REGN2810), BMS-936558, SHR1210, IBI308, PDR001, BGB-A317, BCD-100, JS001, an example of an anti-PD-L2 antibody is sHIgM12. Examples of anti-CTLA-4 antibodies are ipilimumab and tremelimumab (ticilimumab), examples of “anti-LAG-3” antibodies are relatlimab (BMS 986016), Sym022, REGN3767, TSR-033, GSK2831781, MGD013 (bispecific for PD-1 and LAG-3), LAG525 (IMP701), examples of anti-TIM-3 antibodies are TSR-022 and Sym023, and examples of anti-TIGIT antibodies are tiragolumab (MTIG7192A, RG6058) and etigilimab (WO 2018/102536). Preferably the checkpoint inhibitor is an anti-PD-1 antibody, an anti-PD-L1 antibody, an anti-PD-L2 antibody, an anti-LAG-3 antibody, an anti-TIM-3 antibody or an anti-CTLA4 antibody, more preferably an anti-PD-L1 antibody or an anti-PD-1 antibody.

The IL-2/IL-15Rβγ agonist is for use in treating cancer, wherein the cancer is a hematological cancer or a solid cancer. As the mode of action of these agonists is an activation of the innate immune response through activation of NK cells and an activation of the adaptive immune response through activation of CD8⁺ T cells, it is generally assumed that these agonists have great potential to treat both (advanced) solid tumors and hematological malignancies as tested already in numerous murine cancer models and a number of clinical trials in various tumor indications (Robinson and Schluns 2017). Accordingly, IL-2/IL-15Rβγ agonists were tested in colorectal cancer, melanoma, renal cell carcinoma, adenocarcinoma, carcinoid tumor, leiomyosarcoma, breast cancer, ocular melanoma, osteosarcoma, thyroid cancer, cholangiocarcinoma, salivary gland cancer, adenoid cystic carcinoma, gastric cancer, head and neck squamous cell carcinoma, ovarian cancer, urothelial cancer (Conlon, Leidner et al. 2019). ALT-803 was tested in AML and MDS as examples for hematological malignancies (Romee, Cooley et al. 2018). Especially advanced tumor diseases such as metastatic tumors patients may preferably profit from such treatment. In this respect ALT-803 has been tested accordingly in metastatic non-small cell lung cancer (Wrangle, Velcheti et al. 2018). The phase 1/1b clinical trial with SO-C101 is being recruited with patients having renal cell carcinoma, non-small cell lung cancer, small-cell lung cancer, bladder cancer, melanoma, Merkel-cell carcinoma, skin squamous-cell carcinoma, microsatellite instability high solid tumors, triple-negative breast cancer, mesothelioma, thyroid cancer, thymic cancer, cervical cancer, biliary track cancer, hepatocellular carcinoma, ovarian cancer, gastric cancer, head and neck squamous-cell carcinoma, and anal cancer. Examples of hematological cancers are leukemias such as acute lymphoblastic leukemia (ALL), acute myelogenous leukemia (AML), chronic lymphocytic leukemia (CLL), Chronic myelogenous leukemia (CML) and acute monocytic leukemia (AMoL), lymphomas such as Hodkin's lymphomas, Non-Hodkin's lymphomas, and myelomas. With respect to a combination with enfortumab vedotin bladder cancer, urothelial cancer, renal cancer, cervical cancer, endometrial cancer, ovarian cancer, pancreatic cancer lung cancer, prostate cancer, head and neck cancer, esophageal cancer and breast cancer are preferred, especially urothelial cancer, lung cancer, head and neck cancer, pancreatic cancer, renal cancer, breast cancer, cervical cancer and endometrial cancer.

Accordingly, renal cell carcinoma, lung cancer (especially non-small cell lung cancer, small-cell lung cancer), bladder cancer (especially urothelial cancer), melanoma, Merkel-cell carcinoma, skin squamous-cell carcinoma, microsatellite instability high solid tumors, breast cancer (especially triple-negative breast cancer), mesothelioma, prostate cancer, thyroid cancer, thymic cancer, cervical cancer, biliary track cancer, hepatocellular carcinoma, ovarian cancer, gastric cancer, esophageal cancer, head and neck squamous-cell carcinoma, and anal cancer, and ALL, AML, CLL, CML, AMoL, Hodgkin's lymphomas, Non-Hodgkin's lymphomas, and myelomas are preferred cancer indications.

In one embodiment, the IL-2/IL-15Rβγ receptor agonist is administered in a cyclical administration regimen that comprises
(a) a first period of up to three weeks during which the cytotoxic compound capable of inducing ICD is administered,
(b) optionally, a second period without administration of the cytotoxic compound and without administration of the IL-2/IL-15Rβγ agonist,
(c) a third period of up to two weeks during which the IL-2/IL-15Rβγ agonist is administered, wherein the first to third period is repeated at least two times, more preferably at least three times, more preferably up to disease progression.

In one embodiment, the first period is two weeks. The administration of the cytotoxic compound capable of inducing ICD may occur according to its label.

In one embodiment, the second period is a time period of at least the in vivo half-life or at least twice the in vivo half-life of cytotoxic compound capable of inducing ICD.

In one embodiment, the third period is one week.

In one embodiment, the IL-2/IL-15Rβγ receptor agonist is administered in a cyclical administration regiment that comprises
(a) a first period of up to three weeks, preferably for two weeks, more preferably for one week during which the cytotoxic compound capable of inducing ICD is administered according to its label during such first period,
(b) optionally, a second period of at least one or two times the in vivo half-life, preferable of one time the in vivo half-life of the cytotoxic compound without administration of the cytotoxic compound,
(c) a third period of up to two weeks, preferably one week of administration of the IL-2/IL-15Rβγ agonist, and
repeating the first to third period at least two times, more preferably at least three times, more preferably up to disease progression.

Optionally, the cyclical administration regimen further comprises a fourth period (d) without administration of the ICD inducing cytotoxic compound and without administration of the IL-2/IL-15Rβγ agonist having at least one week up to one in vivo half-life of the IL-2/IL-15Rβγ agonist, wherein the fourth period is added after each third period prior to restarting the cycle.

For a combined dosing schedule of the IL-2/IL-15Rβγ agonist with an ICD inducing cytotoxic compound, treatment schedules of both compounds should be aligned in order to obtain best treatment results, in easy, preferably weekly, intervals and being best adjusted to instructions according to labels of approved drugs.

Many chemotherapies, such as anthracyclines, microtubule-destabilizing agents including vinca alkaloids, taxanes, epothilones, eribulin, auristatin, maytansine or maytansinoid, tubulysine, bleomycin, proteasomal inhibitors including bortezomib, alkylating agents including cyclophosphamide, platinum complexes including oxaliplatin, pyrrolo-benzodiazepine, calicheamicin derivatives, topoisomerase I inhibitors, and nucleoside analogues, are typically administered daily over a longer period. In order to obtain the combined effect of inducing ICD with the immune activating effect of the IL-2/IL-15Rβγ agonist, the inventors foresee the treatment with such chemotherapy is applied according to their label, but only up to two weeks, preferably for only one week, to allow for intermittent treatment with the IL-2/IL-15Rβγ agonist.

Indeed, the inventors have shown increased antitumor activity for combined administrations of a platinum complex in combination with an IL-2/IL-15Rβγ agonist, in this case oxaliplatin in combination with SOT101 in the MC38 murine colon carcinoma model in vivo.

With respect to ADCs, many of them are administered every three weeks, given their typical half-life between about 2 to about 12 days. In vivo half-life of ADCs are shown in Table 2 of Mahmood et al. (2021). Kadcyla, Adcetris, Enhertu and Trodelvy are administered in a 3 week/21 days cycle, Padcev is administered in a 4 week cycle (see Table 1).

TABLE 1
dosing schedules of approved ADCs

| | |
|---|---|
| Kadcyla | 3.6 mg/kg given as an i.v. every 3 weeks (21-day cycle) |
| Adcetris | 1.2 mg/kg up to a maximum of 120 mg every 2 weeks until a |
| | maximum of 12 doses, or 1.8 mg/kg up to a maximum of |
| | 180 mg every 3 weeks until a maximum of 16 cycles |
| Padcev | 1.25 mg/kg (up to a maximum of 125 mg for patients 100 kg |
| | or greater) IV over 30 minutes on Days 1, 8. and 15 of a |
| | 28-day cycle until disease progression |
| Enhertu | 5.4 mg/kg given i.v. once every 3 weeks (21-day cycle) until |
| | disease progression, or 6.4 mg/kg given i.v. once every |
| | 3 weeks (21-day cycle) until disease progression |
| Trodelvy | 10 mg/kg administered i.v. once weekly on Days 1 and 8 of |
| | 21-day treatment cycles |

Accordingly, ADCs with a 3 week cycle are administered according to their label at day 1 of the first period (e.g. Kadcyla, Adcetris for its 3 week scheme, Enhertu) or day 1 and 8 (Trodelvy). Padcev with its 4 week cycle is preferably administered according to its label on days 1, 8 and 15.

Prior to the immune activation by the administration with the IL-2/IL-15Rβγ agonist, the cytotoxic compound capable of inducing ICD should be absent or only present in residual amounts in the plasma of the patient in order not to interfere with the induced proliferation of immune cells. Therefore, a treatment break of one or two times the in vivo half-life is introduced to clear the compound from circulation. In case of short-lived chemotherapies such treatment break may be as short as one day, but for convenience for the patient also one week. In a continuous treatment regimen the optional treatment period (b) of at least one or two times the in vivo half-life, preferably of one time the in vivo half-life of the cytotoxic compound without administration of the cytotoxic compound and without administration of the IL-2/IL-15Rβγ agonist is preferred.

ADCs according to their labels anyhow are typically cleared from plasma prior to readministration, as e.g. Kadcyla is given every three weeks with an in-vivo half-life of 4 days. Accordingly, no additional period (b) for clearing is required. Therefore, with respect to ADCs having a three week schedule with administration at day 1, the IL-2/IL-15Rβγ agonist is preferably administered after one or two weeks of treatment break, starting at day 8 or day 15, before the ADC is administered again at day 22 (new day 1). For ADCs administered at days 1 and 8 of a three week cycle, the IL-2/IL-15Rβγ agonist is preferably administered starting day 15, as such more frequently administered ADCs have typically have a rather short half-life (e.g. Todelvy with only 16 h) and therefore are cleared from the plasma within a few days. For ADCs with a four week schedule with dosing at days 1, 8 and 15, the IL-2/IL-15Rβγ agonist treatment is preferably started at day 22.

The IL-2/IL-15Rβγ agonist is administered for up to two weeks, preferably for one week, according to its label/its prior use in most advanced clinical trials. Administration frequency again is dependent on its half-life. IL-2/IL-15Rβγ agonists with a short half-life of hours to 1 day are preferably doses within a treatment week at days 1, 2, 3 and 4, preferably at days 1 and 2; in case of a two week treatment period on days 1, 2, 3, 4, 8, 9, 10, 11, preferably at days 1, 2, 8 and 9 of such treatment period. For example, dosing schedules for SO-C101 are disclosed in WO 2020/234387. Optionally, SO-C101 may be intensely dosed by split administrations at day 1, 2, 8, and 9. IL-2/IL-15Rβγ agonists with a longer half-life are preferably administered only once per treatment week on day 1, or day 1 and day 8 in case of a two week treatment period.

Optionally, an additional treatment break of at least one week, preferable one week, is introduced after each cycle (a) to (c) to allow for sufficient time for the activated immune cells to kill tumor cells.

In case required, the beginning of the new treatment period (a) for the ADC according to its label is delayed by increments of one week to match the time requirements of periods (b), (c) and optionally (d).

Exemplifying dosing schedules of Kadcyla with SO-C101
(a) Administration of Kadcyla at day 1, in a dose of 3.6 mg/kg given i.v.
(b) none
(c) Administration of SO-C101 at days 8, 9, 15, 16 (days 1, 2, 8, 9 of its treatment period), in a daily amount of 12 μg/kg given s.c. (as a single dose or split into two doses with 4 to 14 hours time difference)
(d) none
   or
(a) Administration of Kadcyla at day 1, in a dose of 3.6 mg/kg given as an i.v.
(b) none
(c) Administration of SO-C101 at days 15, 16 (days 1, 2 of its treatment period), in a daily amount of 12 μg/kg given s.c. (as a single dose or split into two doses with 4 to 14 hours time difference)
   or
(a) Administration of Kadcyla at day 1, in a dose of 3.6 mg/kg given as an i.v.
(b) none
(c) Administration of SO-C101 at days 15, 16, 22, 23 (days 1, 2, 8, 9 of its treatment period), in a daily amount of 12 μg/kg given s.c. (as a single dose or split into two doses with 4 to 14 hours time difference)
(d) 1 week
   thereby extending the Kadcyla schedule to four weeks starting again at day 29.

Another embodiment is the use of an IL-2/IL-15Rβγ agonist in the manufacture of a kit of parts for the treatment of cancer, wherein the kit of parts comprises:

several doses of the IL-2/IL-15Rβγ agonist of the invention, an instruction for administration of such IL-2/IL-15Rβγ agonist in combination with a cytotoxic compound capable of inducing ICD and/or a modality capable of inducing ICD, and optionally an administration device for the IL-2/IL-15Rβγ agonist. In a preferred embodiment the kit further comprises a checkpoint inhibitor and an instruction for use of the checkpoint inhibitor.

The invention also involves methods of treating cancer involving the above described combined treatments, as well as methods for stimulating NK cells and/or CD8⁺ T cells involving the above described combined treatments.

In one embodiment, the invention relates to an IL-2/IL-15Rβγ agonist for use in treating cancer in a patient, wherein said IL-2/IL-15Rβγ agonist is administered in combination with a cytotoxic compound capable of inducing ICD.

In one embodiment, the invention relates to an IL-2/IL-15Rβγ agonist for use in treating cancer in a patient, wherein said IL-2/IL-15Rβγ agonist is administered in combination with an application of a modality capable of inducing ICD.

The present invention also provides a pharmaceutical combination comprising an IL-2/IL-15Rβγ agonist and a cytotoxic compound capable of inducing ICD.

The present invention further provides a pharmaceutical combination comprising an IL-2/IL-15Rβγ agonist and a modality capable of inducing ICD.

The administration of the IL-2/IL-15Rβγ agonist may occur simultaneously or sequentially to the administration of the cytotoxic compound capable of inducing ICD and/or to the application of a modality capable of inducing ICD.

In one embodiment, the IL-2/IL-15Rβγ agonist is SOT101 and the cytotoxic compound capable of inducing ICD is Kadcyla.

In one embodiment, the IL-2/IL-15Rβγ agonist is SOT101 and the cytotoxic compound capable of inducing ICD is an ADC comprising an anti-CLDN18.2 antibody and an anthracycline.

In one embodiment, the IL-2/IL-15Rβγ agonist is SOT101 and the cytotoxic compound capable of inducing TCD is SOT102.

In one embodiment, the IL-2/IL-15Rβγ agonist is SOT101 and the modality capable of inducing ICD is radiation therapy. In a specially preferred embodiment, the IL-2/IL-15Rβγ agonist is SOT101 and the modality capable of inducing ICD is non-ablative or sub-ablative radiation therapy.

In one embodiment, the IL-2/IL-15Rβγ agonist is SOT101 and the cytotoxic compound capable of inducing ICD is gemtuzumab ozogamicin.

In one embodiment, the IL-2/IL-15Rβγ agonist is SOT101 and the cytotoxic compound capable of inducing ICD is brentuximab vedotin.

In one embodiment, the IL-2/IL-15Rβγ agonist is SOT101 and the cytotoxic compound capable of inducing ICD is trastuzumab emtansine.

In one embodiment, the IL-2/IL-15Rβγ agonist is SOT101 and the cytotoxic compound capable of inducing ICD is inotuzumab ozogamicin.

In one embodiment, the IL-2/IL-15Rβγ agonist is SOT101 and the cytotoxic compound capable of inducing ICD is trastuzumab deruxtecan.

In one embodiment, the IL-2/IL-15Rβγ agonist is SOT101 and the cytotoxic compound capable of is inducing ICD is enfortumab vedotin.

In one embodiment, the IL-2/IL-15Rβγ agonist is SOT101 and the cytotoxic compound capable of inducing ICD is polatuzumab vedotin.

In one embodiment, the IL-2/IL-15Rβγ agonist is SOT101 and the cytotoxic compound capable of inducing ICD is sacituzumab govitecan.

In one embodiment, the IL-2/IL-15Rβγ agonist is SOT101 and the cytotoxic compound capable of inducing ICD is Belantamab mafodotin-blmf.

In one embodiment, the IL-2/IL-15Rβγ agonist is SOT101 and the cytotoxic compound capable of inducing ICD is Loncastuximab tesirine-lpyl.

In one embodiment, the IL-2/IL-15Rβγ agonist is SOT101 and the cytotoxic compound capable of inducing ICD is Tisotumab vedotin-tftv.

In one embodiment, the IL-2/IL-15Rβγ agonist is SOT101 and the cytotoxic compound capable of inducing ICD is an anthracycline, preferably doxorubicin.

In one embodiment, the IL-2/IL-15Rβγ agonist is SOT101 and the cytotoxic compound capable of inducing ICD is a taxan, preferably paclitaxel.

In one embodiment, the IL-2/IL-15Rβγ agonist is SOT101 and the cytotoxic compound capable of inducing ICD is bortezomib.

In one embodiment, the IL-2/IL-15Rβγ agonist is SOT101 and the cytotoxic compound capable of inducing ICD is a platinum complex, preferably oxaliplatin or cisplatin, more preferably oxaliplatin.

In one embodiment, the IL-2/IL-15Rβγ agonist is SOT101 and the cytotoxic compound capable of inducing ICD is a topotecan or exatecan.

In one embodiment, the IL-2/IL-15Rβγ agonist is SOT101 and the cytotoxic compound capable of inducing ICD is gemcitabine.

In one embodiment, the IL-2/IL-15Rβγ agonist is SOT101 and the cytotoxic compound capable of inducing ICD is cyclophosphamide.

In a preferred embodiment the cytotoxic compounds are dosed at a lower dosage and/or less frequently compared to the label for use in cancer treatment.

### FIGURES

FIG. 1: Antitumor efficacy of chemotherapy. Chemotherapeutic agents activate molecular pathways that elicit upregulation and/or release of stress molecules (danger-associated molecular patterns—DAMPs; NK cell ligands . . . ) that promote tumor cell recognition and elimination by NK cells. Moreover, chemotherapy can also downregulate the expression of ligands such as PD-L1 and (MHC)-I of inhibitory receptors.

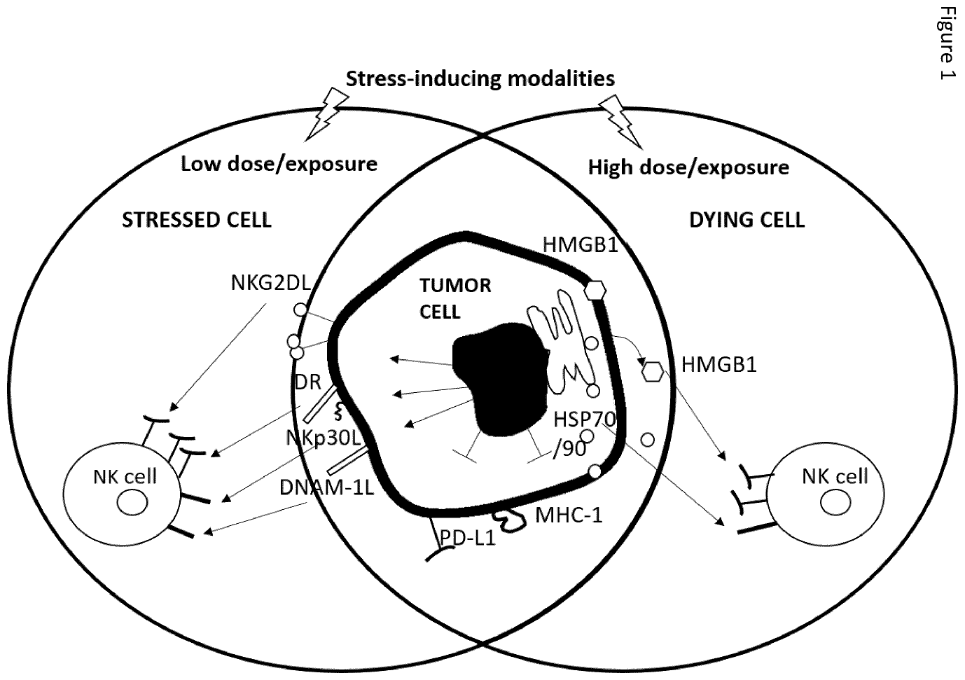

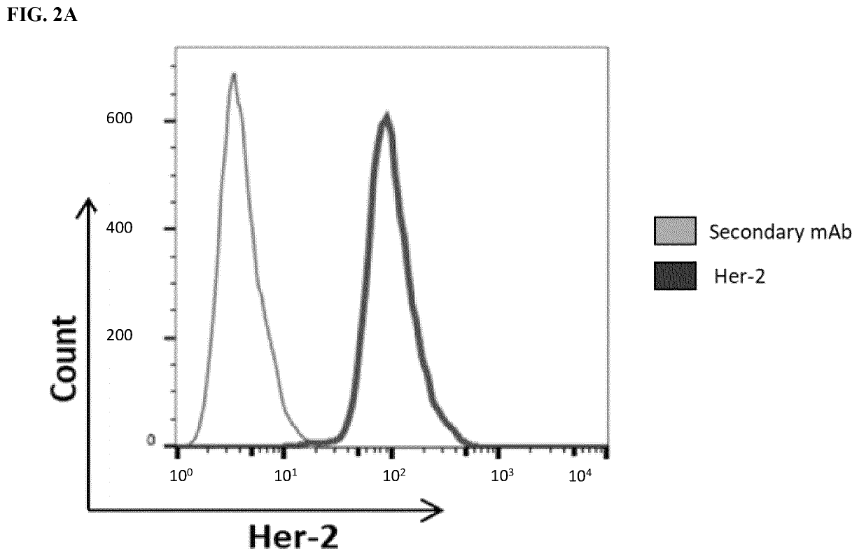

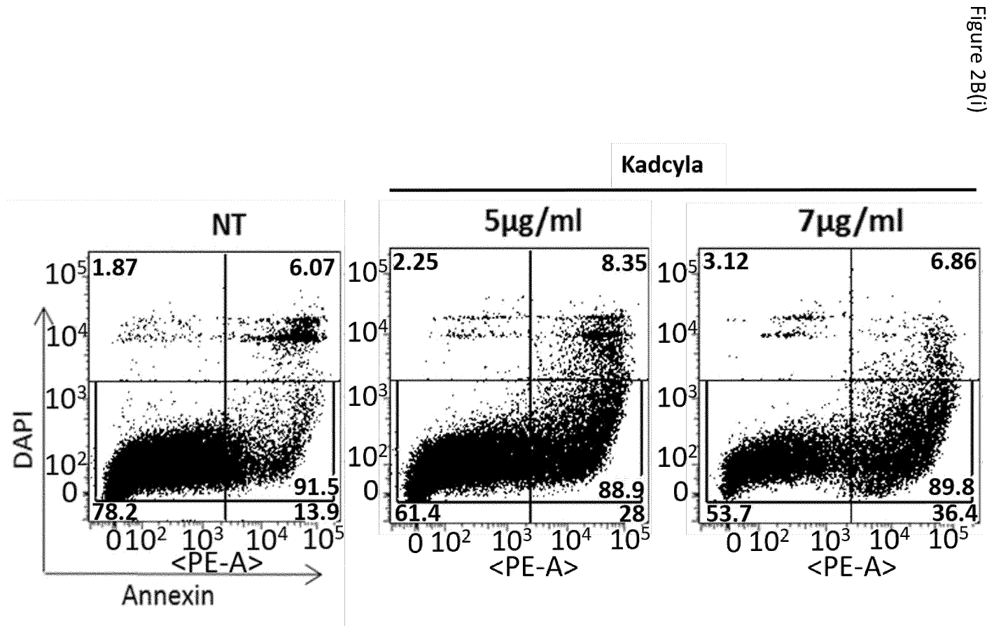

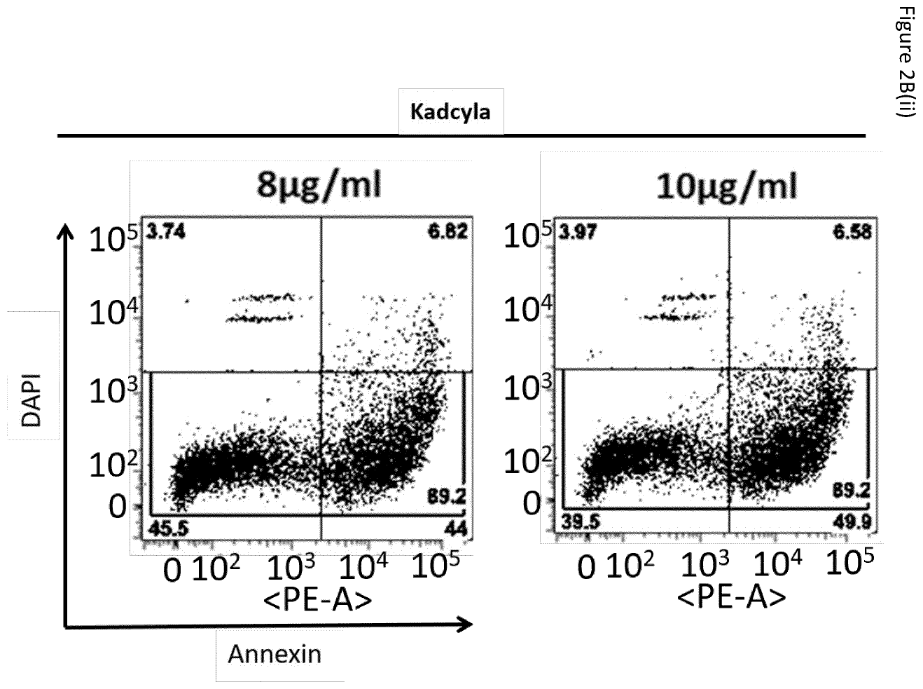

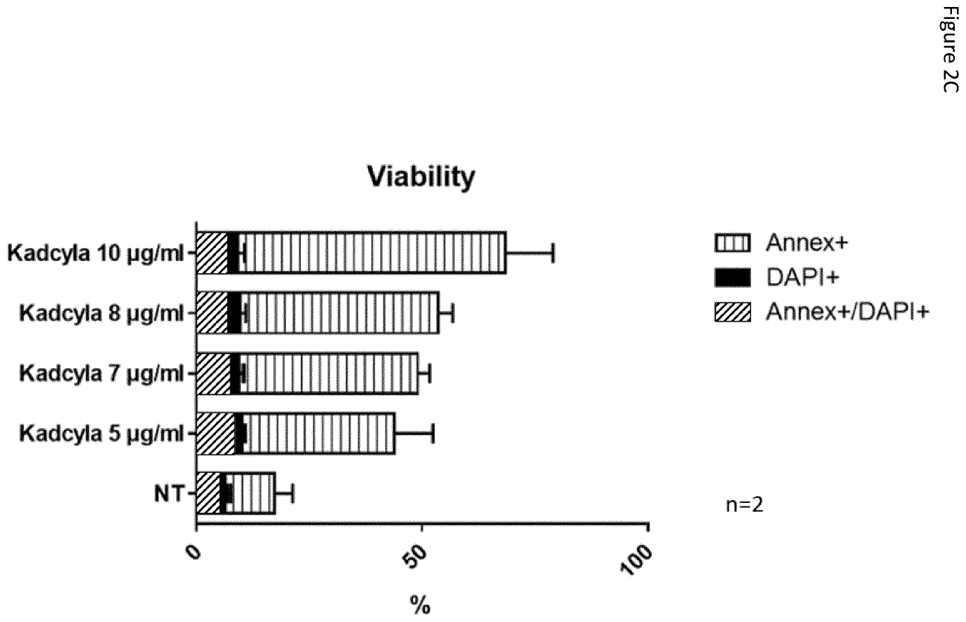

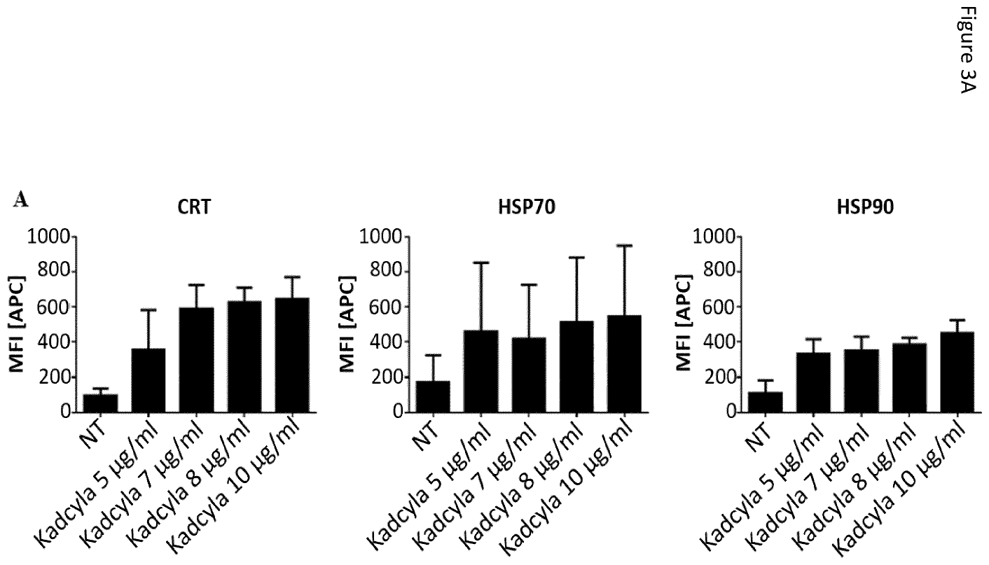

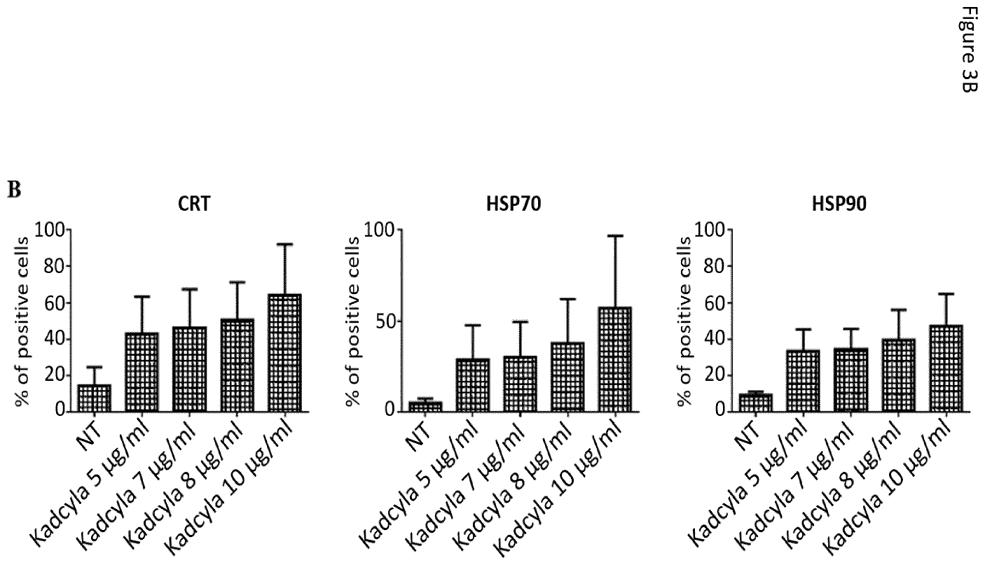

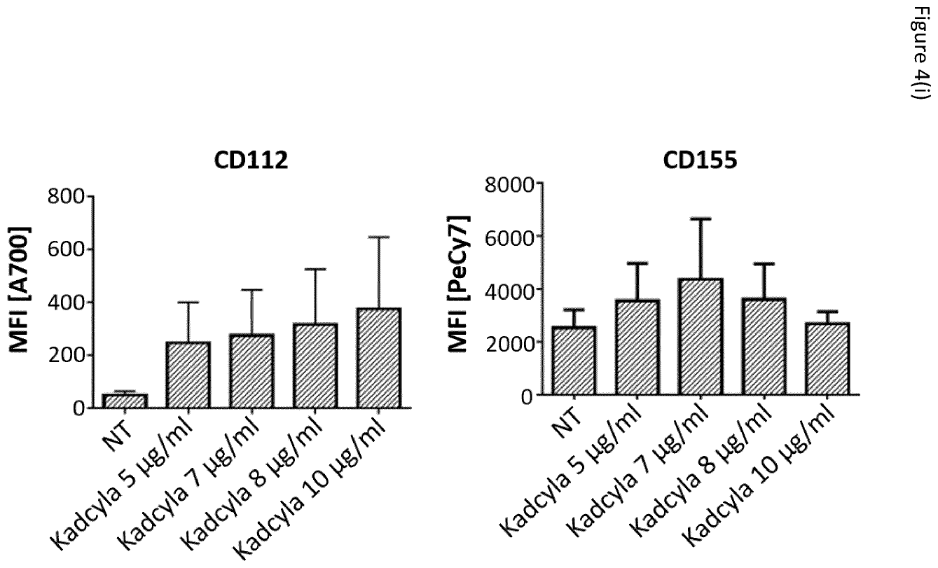

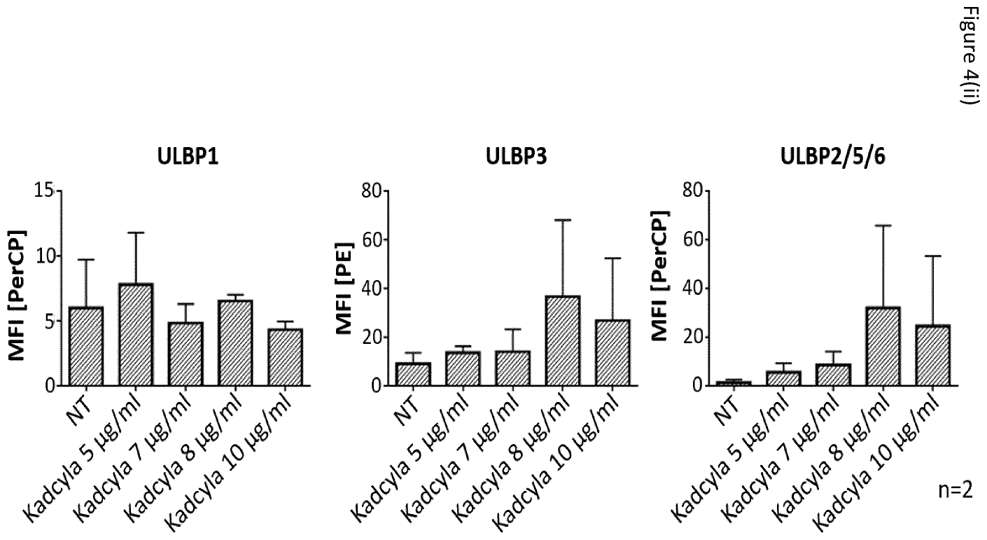

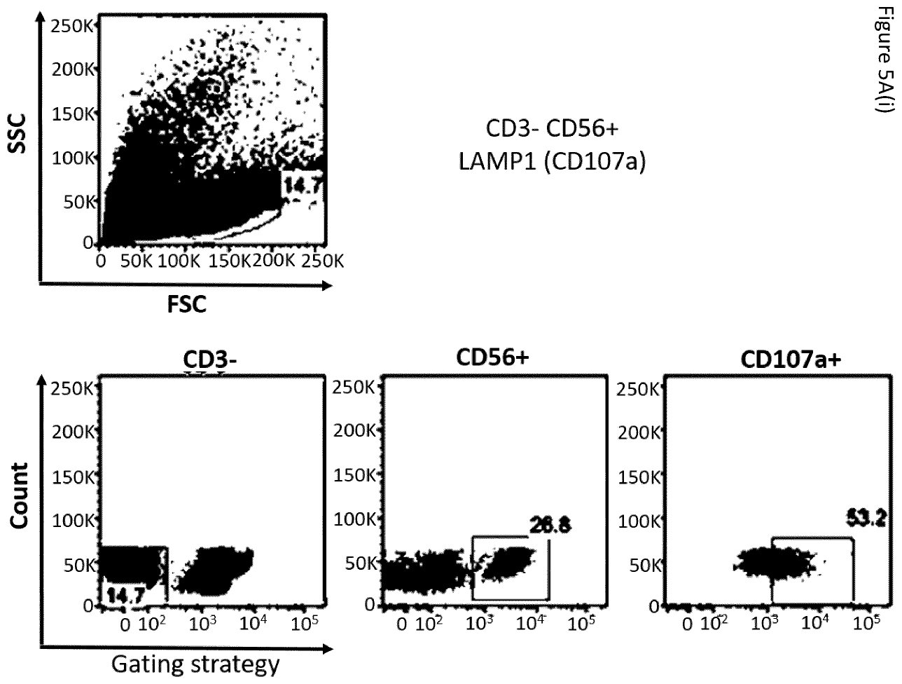

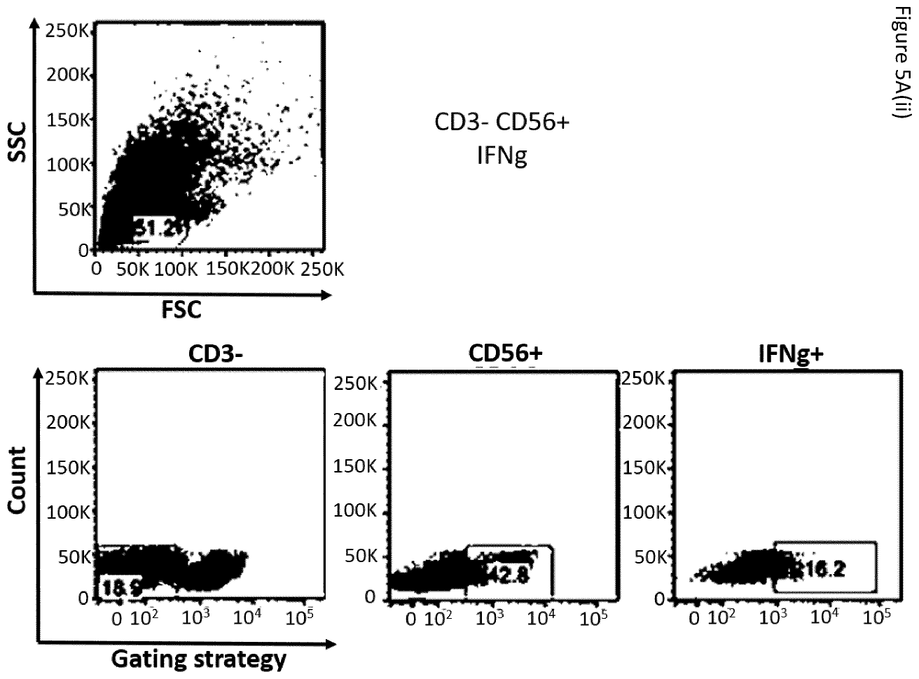

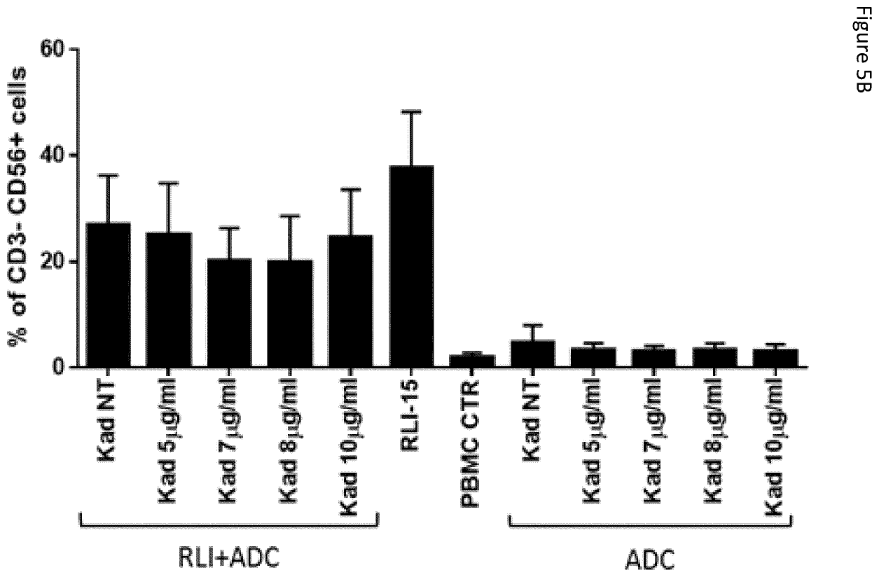

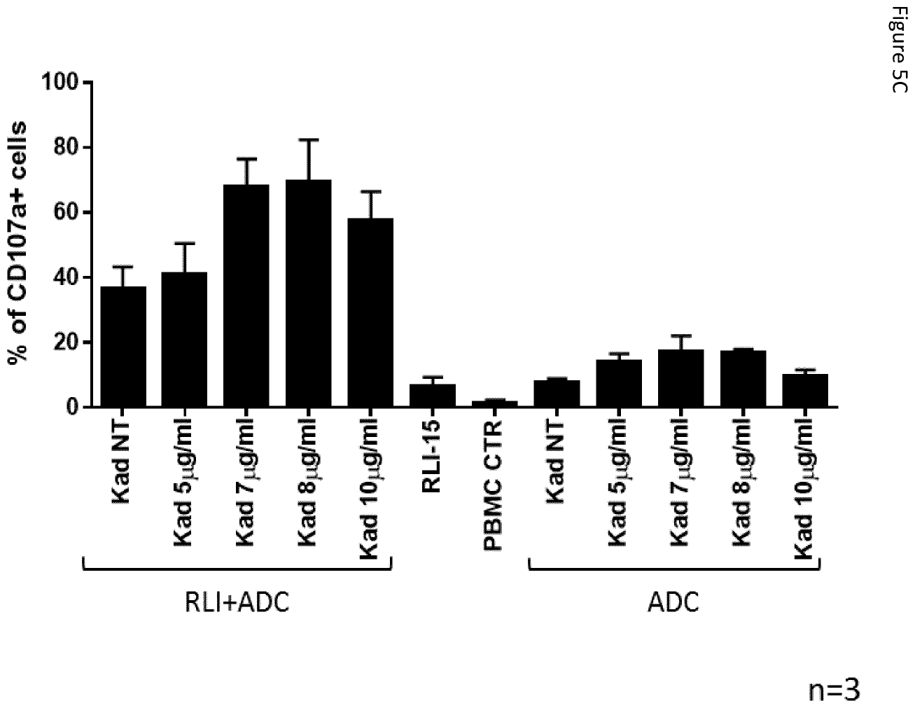

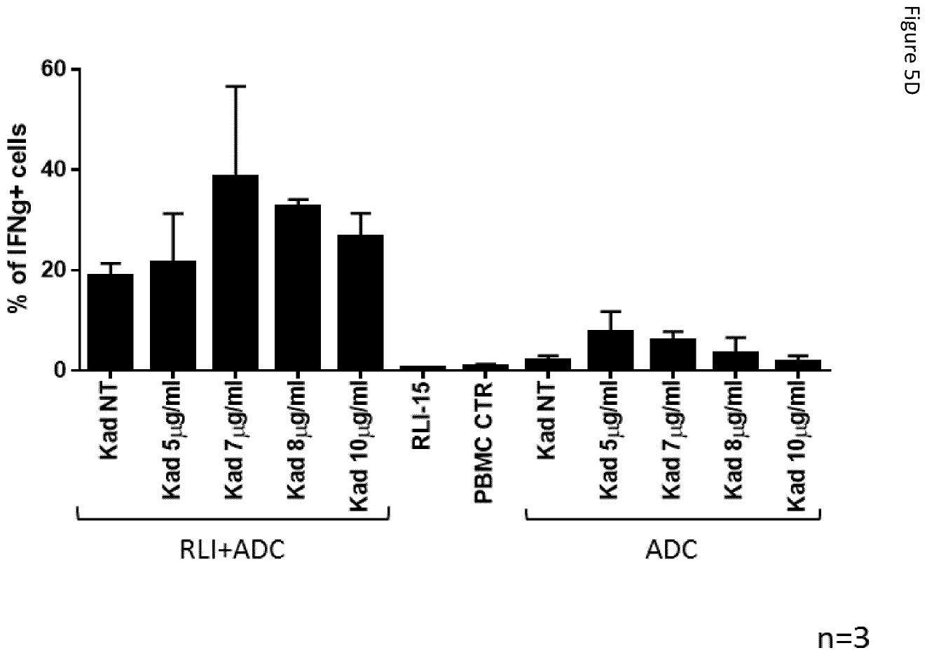

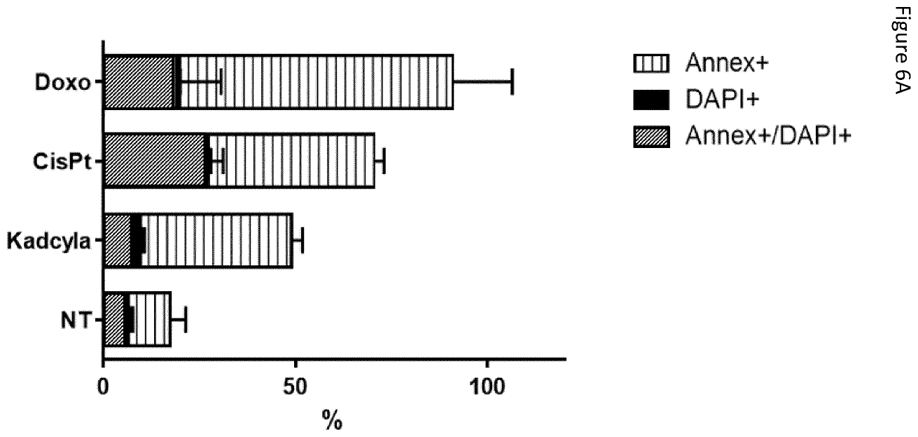

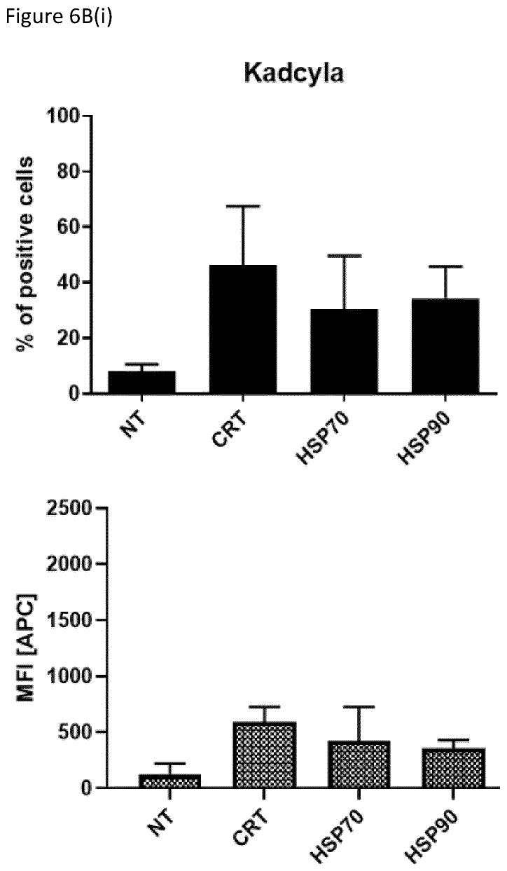

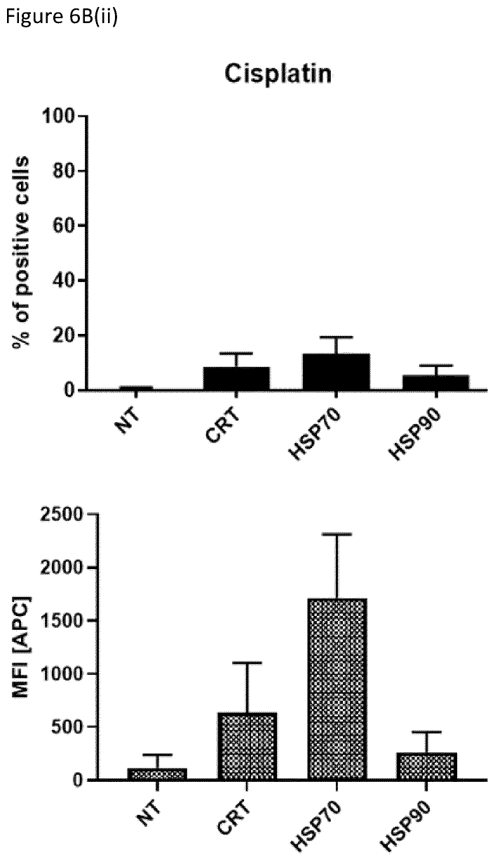

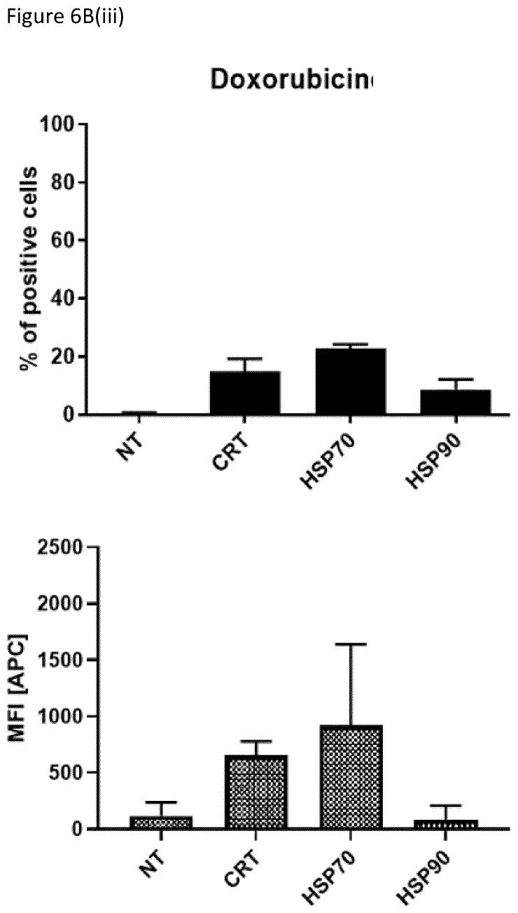

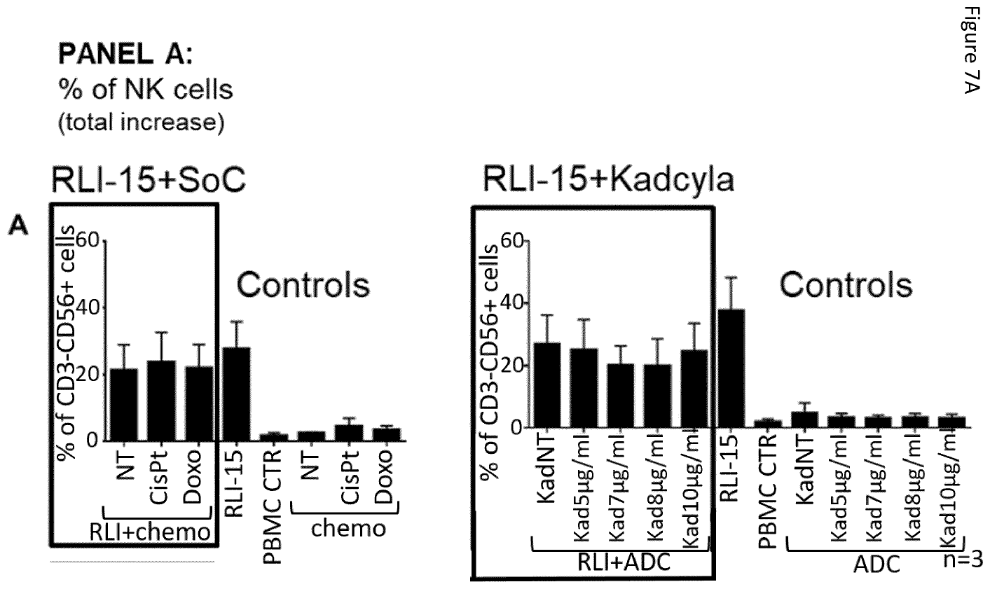

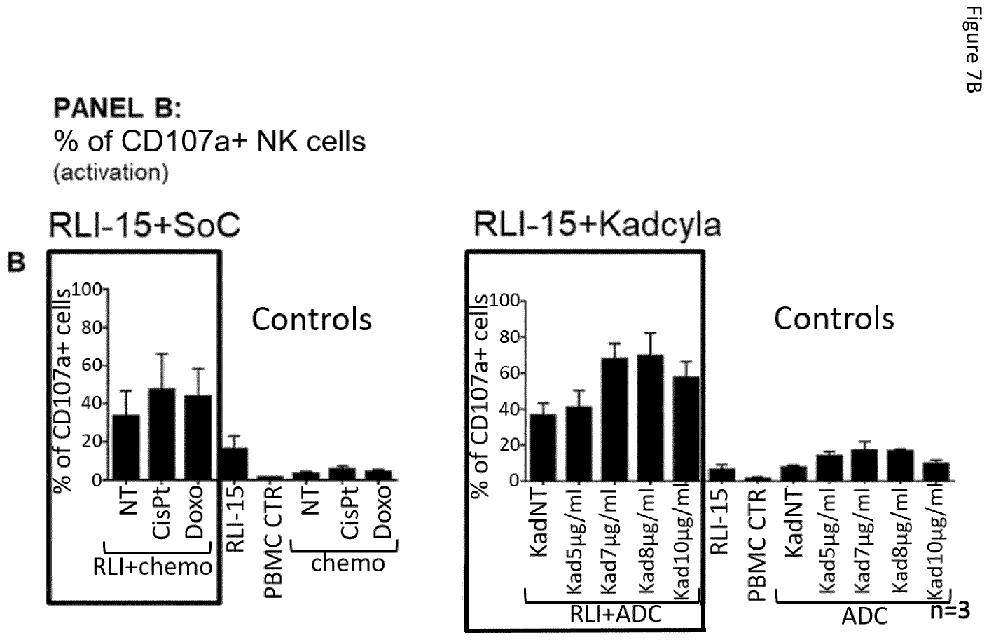

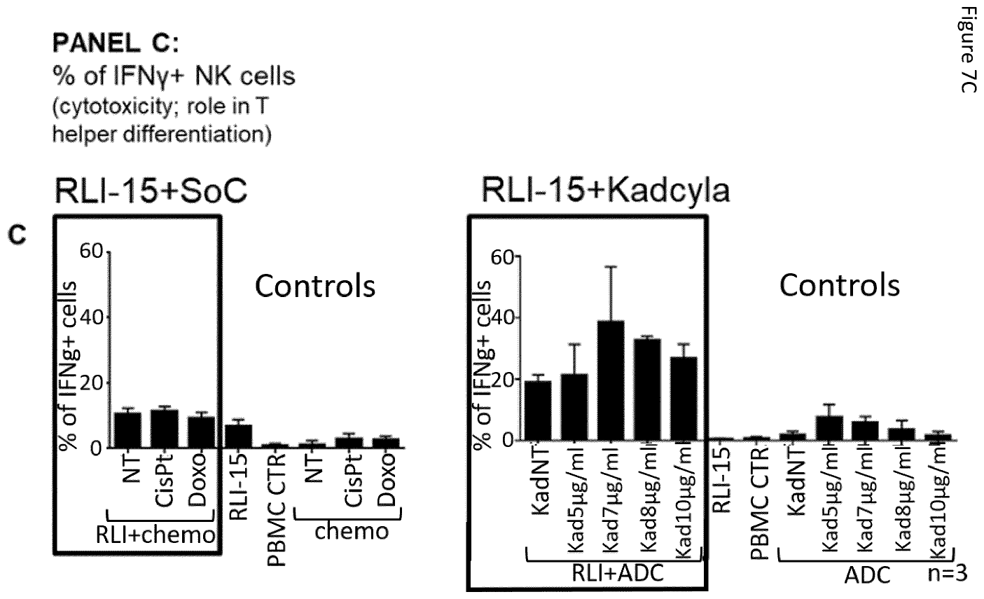

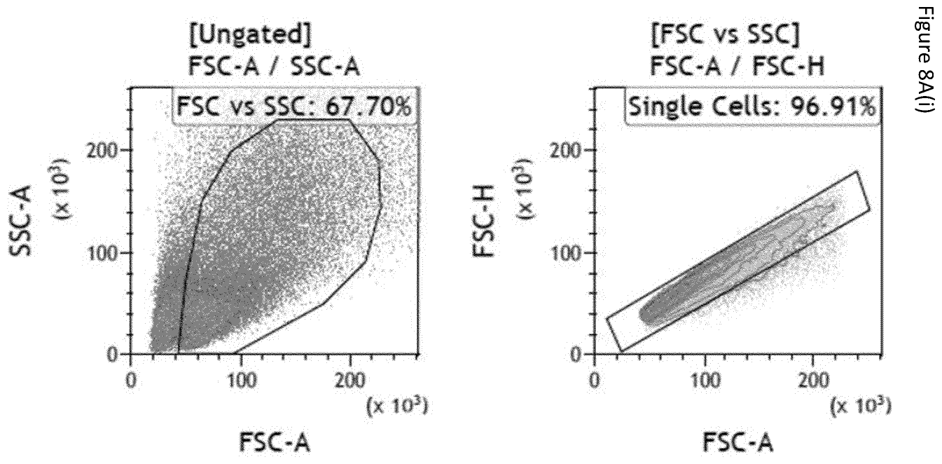

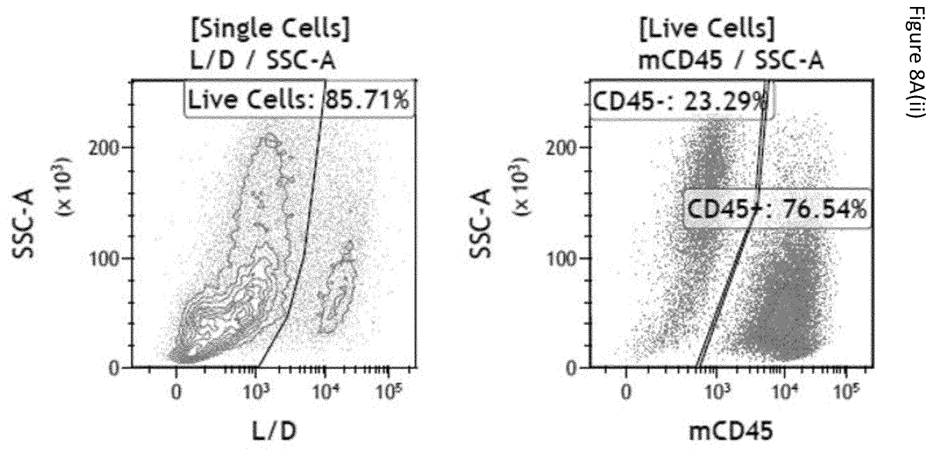

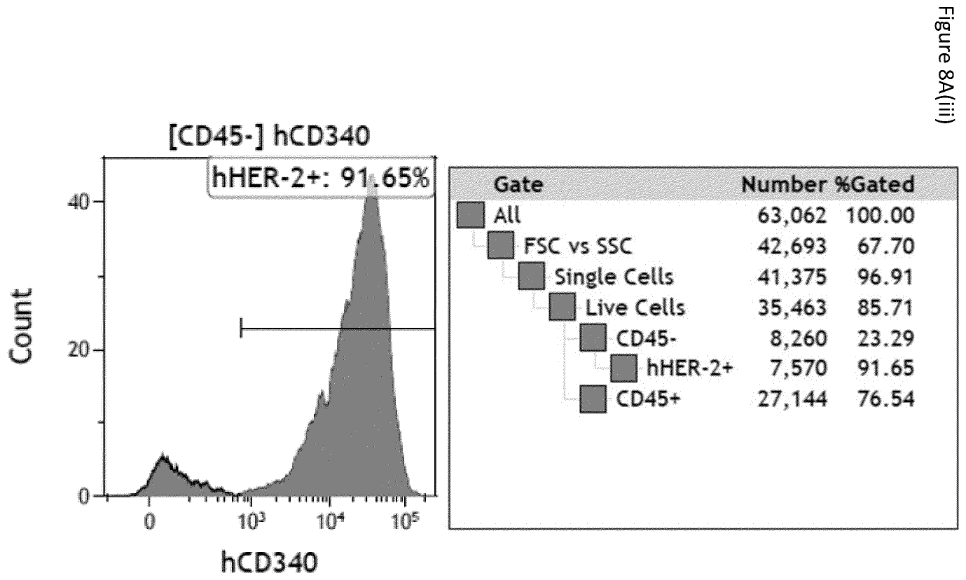

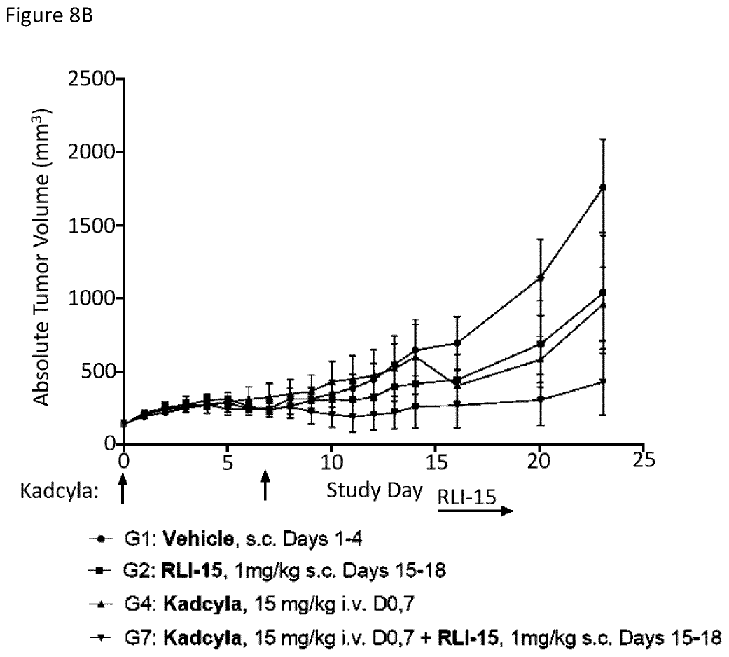

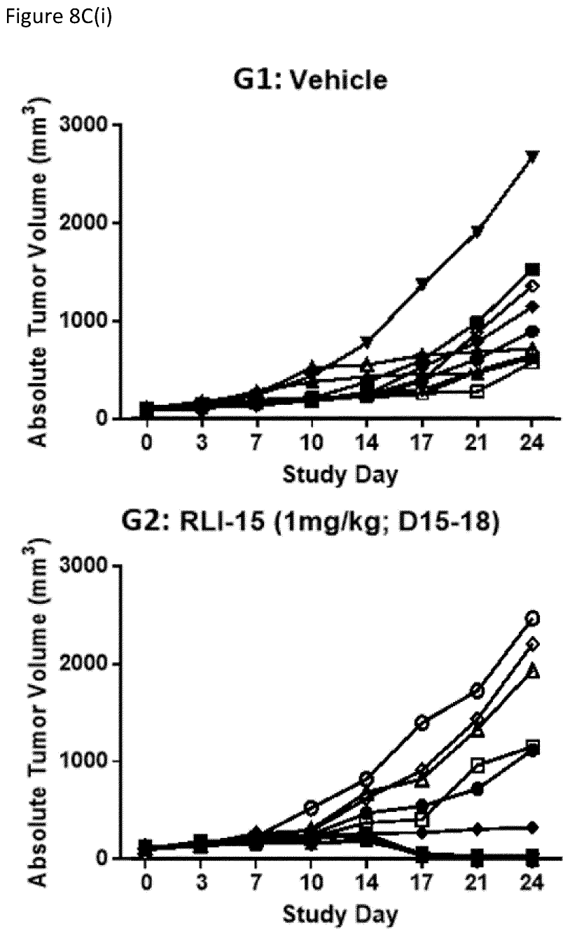

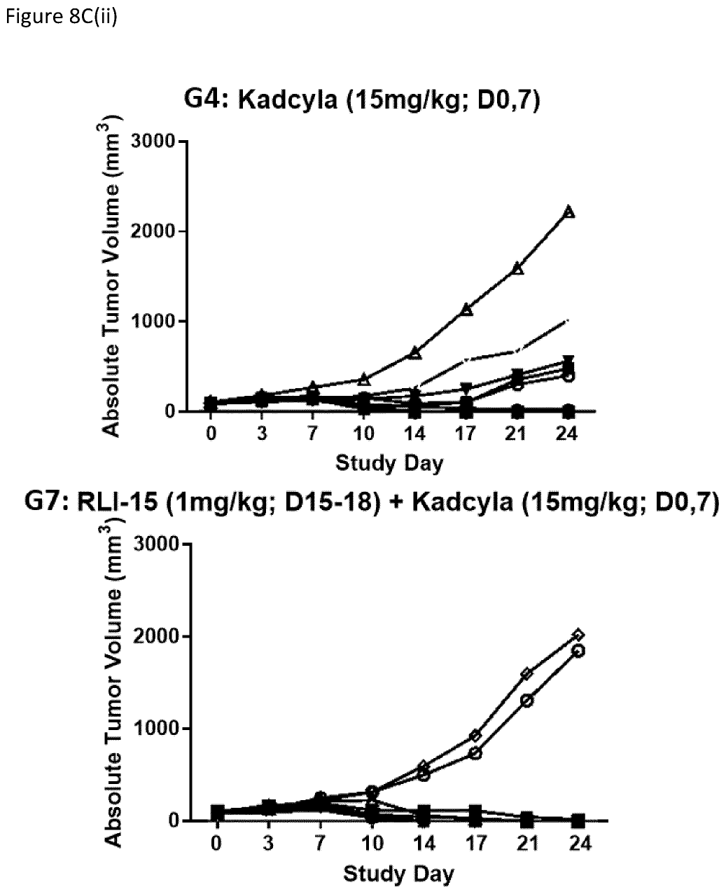

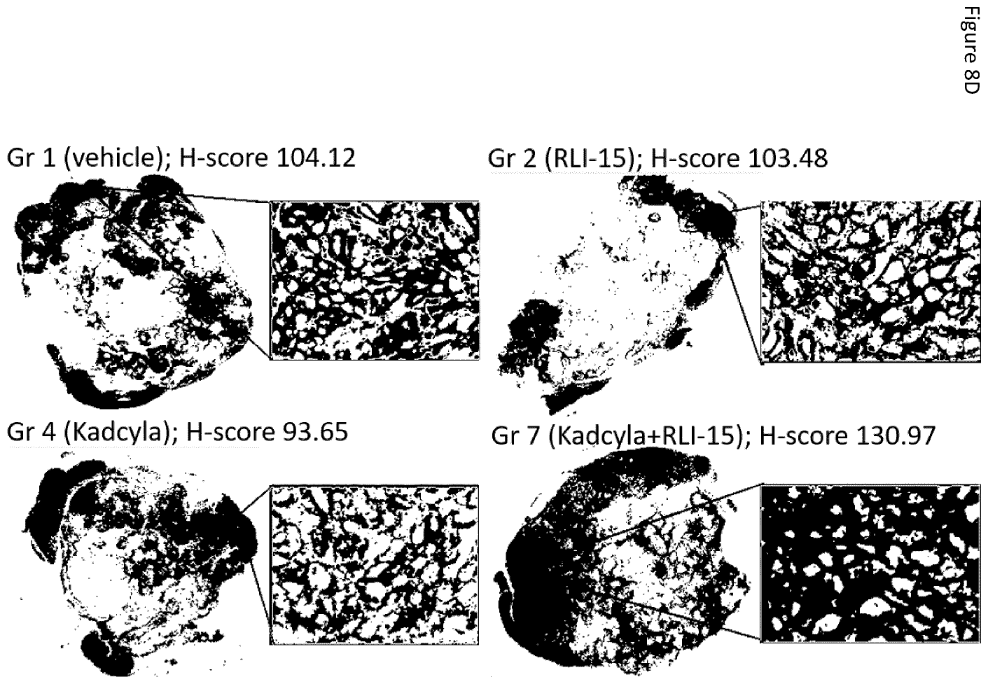

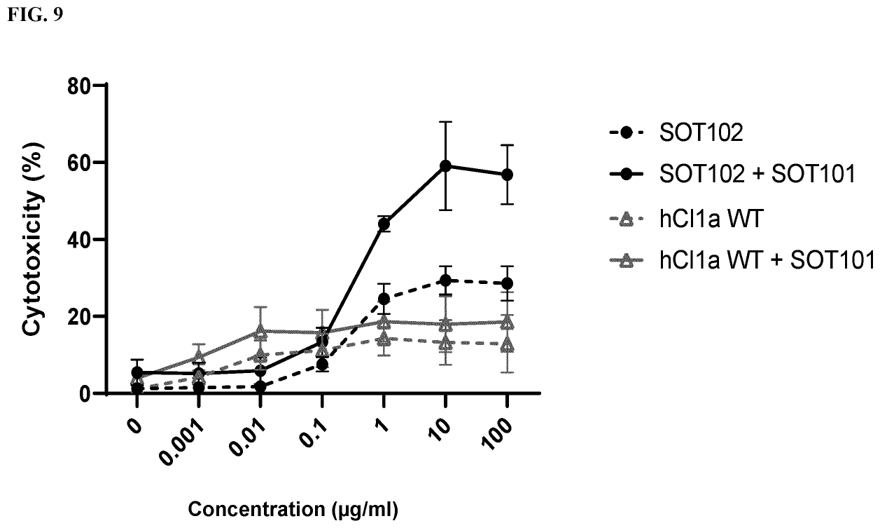

FIG. 2: Dose-dependent cytotoxic effect of Kadcyla in AGS tumor cell line. Gastric adenocarcinoma cell line (AGS) was treated with indicated (5, 7, 8, 10 μg/ml) concentration of Kadcyla for 72 h. Data are representing mean of two independent experiments.
(A) Cell surface expression of Her-2 analyzed by FACS on non-treated AGS human gastric adenocarcinoma cell line (Her-2 IHC1+).
(B) Viability of tumor cells on endpoint was analyzed by FACS analysis using AnnexinV (x axis)/DAPI (y axis) staining. Representative dot plots are shown with NT for not-treated, Kadcyla concentrations of 5 μg/ml, 7 μg/ml, 8 μg/ml and 10 μg/ml.
(C) Individual populations of early apoptotic (Annex⁺/DAPI⁻—hatched), late apoptotic (Annex⁺/DAPI⁺—grey) and necrotic (Annex⁻/DAPI⁺—black) cells at the given concentration. For the analysis of ICD markers and NK ligands only early apoptotic cells (Annex⁺/DAPI⁻ population) were selected.

FIG. 3: Dose-dependent induction of cell surface exposure of CRT, HSP70, HSP90 in AGS tumor cell line by Kadcyla. Population of early apoptotic (Annex⁺/DAPI⁻) from FIG. 2 was analyzed for the expression of CRT, HSP70 and HSP90 on the cell surface. Data are representing mean of two independent experiments.
(A) Mean fluorescence intensity (MFI) of individual markers CRT, HSP70 and HSP90 of antigen presenting cells (APC).
(B) Correlating percentage of binding of individual primary antibodies to individual markers CRT, HSP70 and HSP90.

FIG. 4: Dose-dependent induction of expression of NK cell ligands on the surface of AGS tumor cell line by Kadcyla. Population of early apoptotic (Annex⁺/DAPI⁻) from FIG. 2 was analyzed for the expression of NK activation ligands CD112, CD155 and ULBP1, 2/5/6 and 3 and expressed as a mean fluorescence intensity (MFU). Data are representing mean of two independent experiments.

FIG. 5: Dose-driven in vitro synergy of RLI-15 to Kadcyla on stimulation of CD56-positive cells. Human PBMCs isolated from three different donors were incubated for 72 h in the presence of 2.5 ng/ml of RLI-15. In parallel, AGS tumor cells were treated with indicated (5, 7, 8, 10 μg/ml) concentrations of Kadcyla for 72 h and washed before the two cell cultures were mixed in the ratio of 10 (PBMCs):1 (tumor cell) and incubated for the next 4 h. Subsequently, the whole population was analyzed by flow cytometry for markers CD3, CD56, CD107a and IFNγ.
(A) Representative gating strategy is shown.
(B) % of CD3⁻CD56⁺ cells representing NK cells for increasing concentrations of Kadcyla from 0 (KadNT) to 10 μg/ml Kadcyla, in combination with RLI-15 (RLI+ADC) or only Kadcyla (ADC) with controls of RLI-15 only and untreated PBMCs (PBMC CTR) and
(C) their activation measured by release of CD107a (LAMP1) (C).
(D) their activation measured by release of IFNγ.

The gating strategy is shown in panel A. RLI-15 treatment leads to an increase of total number of CD3−CD56⁺ cells (B). Data are representing mean of three independent experiments.

TABLE 2
Legend to FIG. 5.

| Marked as | Representing | Concentration | |
|---|---|---|---|
| — | PBMC | — | |
| RLI | RLI-15 | 2.5 | ng/ml |

| | | |
|---|---|---|
| Kad | ADC (Kadcyla) | NT |

| | |
|---|---|
| 5 | μg/ml |
| 7 | μg/ml |
| 8 | μg/ml |
| 10 | μg/ml |

| | | |
|---|---|---|
| — | Tumor cells (AGS) | — |

FIG. 6: Direct comparison of the cytotoxic effect caused by selected ICD-inducing SoC (doxorubicin, cisplatin) and Kadcyla in AGS tumor cell line. Gastric tumor cell line (AGS) was treated for 48 h with a defined concentration (titration data not shown) of doxorubicin or cisplatin (Table 3), respectively, or for 72 h with the previously defined concentration of Kadcyla with the highest potential to synergize with RLI-15 in vitro (FIG. 6B-D) (Table 3). Data are representing the mean of two independent experiments.

TABLE 3
Concentrations used for the treatment of the AGS cell
line to compare the synergistic potential of selected
SoC (doxorubicin, cisplatin) and Kadcyla to RLI-15.

| | | |
|---|---|---|
| Treatment | Concentration | Timepoint |

| | | | |
|---|---|---|---|
| Doxorubicin | 1.2 | μM | 48 h |
| Cisplatin | 100 | μM | |
| Kadcyla | 7 | μg/ml | 72 h |

(A) Individual populations of early apoptotic (Annex⁺/DAPI⁻—hatched), late apoptotic (Annex⁺/DAPI⁺—grey) and necrotic (Annex⁻/DAPI⁺—black) cells at given concentrations of SoC and Kadcyla (Table 3).
(B) Induction of cell surface exposure of CRT, HSP70, HSP90 in AGS tumor cell line by SoC and Kadcyla. Population of early apoptotic (Annex⁺/DAPI⁻) from
   FIG. 6A was analyzed for the expression of CRT, HSP70 and HSP90 on the cell surface. Mean fluorescence intensity (MFI) of individual markers CRT, HSP70 and HSP90 of antigen presenting cells (APC) and correlating percentage of binding of individual primary antibodies to individual markers CRT, HSP70 and HSP90. Data are representing mean of two independent experiments.

FIG. 7: Comparison of the in vitro synergy of RLI-15 to SoC (doxorubicin, cisplatin) or Kadcyla, respectively, on stimulation of CD56-positive cells. Human PBMCs isolated from three different donors were incubated for 72 h in the presence of 2.5 ng/ml of RLI-15. In parallel, AGS tumor cells were treated either with a defined concentration of SoC (doxorubicin or cisplatin, respectively) for 48 h or with 5, 7, 8 or 10 μg/ml Kadcyla for 72 h (as before, see Table 2), washed before the two cell cultures were mixed in the ratio of 10 (PBMCs):1 (tumor cell) and incubated for the next 4 h. Subsequently, the whole cell population was analyzed by flow cytometry for markers CD3, CD56, CD107a and IFNγ.
Panel A: % of CD3⁻CD56⁺ cells representing the total NK cell fraction in the samples treated with SoC (chemo: Doxo for doxorubicin, CisPt for cisplatin) or Kadcyla (Kad, ADC) only, or in combination with RLI-15 (RLI-15+chemo; RLI-15+ADC), marked as controls.
Panel B: % of activated NK cells measured by release of CD107a (LAMP1)
Panel C: % of cytotoxic NK cells measured by release of IFNγ.

The gating strategy was similar to that shown in FIG. 5 A. Data are representing mean of three independent experiments.

FIG. 8: Antitumor efficacy of RLI-15 in combination to Kadcyla in mouse orthotopic huHER2 EMT-6 breast cancer model. Groups of n=8 animals (Balb/c AnN, fully immunocompetent mice) were implanted with EMT-6 breast cancer cell line with an engineered human HER2 receptor. Treatment with Kadcyla (15 mg/kg; Day 0, 7) was initiated once the tumors reached mean tumor volume of 140 mm³ in individual groups. Two weeks later, animals were given RLI-15 (1 mg/kg; Day 15-18) and results were evaluated on study Day 23.
(A) Representative example of the ex-vivo Her-2 expression FC analysis in the tumor of one of the study mice (total n=3 mice were euthanized at the randomization stage and subjected to ex vivo analysis of Her-2 tumor expression).
(B) Mean absolute tumor volume (mm³) with SEM dependent on time shown as study days for treatment groups. G1: Vehicle, s.c. administration at days 1-4; G2: RLI-15, s.c. administration of 1 mg/kg on days 15-18; G4: i.v. administration of Kadcyla at days 0 and 7; and G7: i.v. administration of Kadcyla at days 0 and 7, and RLI-15, s.c. administration of 1 mg/kg on days 15-18. Vertical arrows show single i.v. administrations of Kadcyla. The horizontal arrow shows the 4 s.c. administration days of RLI-15.
(C) Individual animal data are shown for the 4 treatment groups G1, G2, G4 and G7 of panel B.
(D) HER2 expression in the huHER2/EMT-6 model in Balb/c mice is at the intermediate level. Paraffin-embedded sections have been prepared from residual tumors of all study mice and stained using HercepTest™. Individual pictures are representatives of staining patterns closest to the mean H-score in individual treatment groups G1, G2, G4 and G7.

FIG. 9: Cell killing assay in vitro of anti-CLDN18.2 ADC in combination with SOT101. A549-CLDN18.2 cells—expressing Claudin 18.2—were incubated with indicated concentrations of the anti-CLDN18.2 antibody hCl1a WT (having an unmodified IgG1 Fc) or SOT102 (an hCl1a-derived ADC with PNU as toxin and LALA Fc IgG1 Fc substitutions) along with freshly isolated human NK cells at an E:T ratio of 10:1. SOT101 was added where indicated to reach 0.1 nM concentration. After 24 h cytotoxicity was measured using lactate dehydrogenase assay (LDH). Data are plotted in % cytotoxicity compared to cells permeabilized with lysis buffer as an average±SEM. n=2.

### EXAMPLES

### Example 1—General Methods

### Flow Cytometric Analysis of HSP70, HSP90 and CRT on the Cell Surface (Fucikova, Moserova et al. 2014):

A total of 1×10⁶ cells are plated in 12-well plates and then treated with the ICD inducing compound or modality for 6, 12 or 24 hr. The cells are collected and washed twice with PBS. The cells are then incubated for 30 min with primary antibody diluted in cold blocking buffer (2% fetal bovine serum in PBS), followed by washing and incubation with an Alexa 648-conjugated monoclonal secondary antibody in blocking solution. Each sample was then analyzed using a FACScan Aria (BD Bioscience). Cell surface expression of HSP70, HSP90 and CRT is analyzed on non-permeabilized annexin V-positive/DAPI-negative cells.

### Detection of HMGB1 Release (Fucikova, Moserova et al. 2014):

After treatment of cells with ICD inducing compounds or modalities, supernatants are collected at different time points (6, 12, 24 and 48 h). Dying tumor cells were removed by centrifugation, and the supernatants were isolated and frozen immediately. Quantification of HMGB1 in the supernatants can be assessed using an enzyme-linked immunosorbent assay according to the manufacturer's instructions (IBL, Hamburg, Germany).

### ATP Detection (Adkins, Sadilkova et al. 2017):

For measurement of extra-cellular ATP release cell culture supernatant is used, and for intracellular ATP detection, cells are centrifuged 2,200 rpm, 2 min and pellet resuspended in cell lysis buffer (eBioscience). ATP content can be determined according to manufacturer's instructions (ATP assay kit, Sigma-Aldrich).

### Example 2—In Vitro Killing of AGS Tumor Cells by Kadcyla

5 MIL cells (seeded in 75 cm² culture flasks) of the gastric AGS tumor cell line (HER2 FC:1-2+) were incubated in the presence of increasing concentrations of Kadcyla (5, 7, 8, 10 μg/ml) for 72 h.

The viability of the tumor cells upon treatment with Kadcyla was analyzed by flow cytometry using AnnexinV (Exbio, Czech Republic) and DAPI dilactate (Thermo-Fisher Scientific, USA) staining for analysis of the amount of living (AnnexinV⁻/DAPI⁻), early apoptotic (AnnexinV⁺/DAPI⁻), late apoptotic (AnnexinV⁺/DAPI⁺) and necrotic cell populations (AnnexinV⁻/DAPI⁺) (see FIGS. 2A and B). Increasing concentrations of Kadcyla led to increasing populations of AnnexinV⁺/DAPI⁻ cells representing the early apoptotic cell population.

The expression of ICD markers Hsp70, Hsp90 and CRT as well as NK cells ligands was measured by flow cytometry on these early apoptotic (AnnexinV⁺/DAPI⁻) cell populations using anti-calreticulin antibody (Abcam, USA), anti-HSP70 (R&D Systems, USA), anti-HSP90 (Enzo Life Sciences, USA). APC AffiniPure F(ab′)₂ Fragment Goat Anti-Mouse (Jackson ImmunoResearch) was used as a secondary antibody. For all tested ICD markers Kadcyla treatment led to a strong increase of ICD markers compared to non-treated cells. Whereas there was no or only a weak trend that the mean fluorescence intensity increased with increasing concentrations of Kadcyla, this trend was stronger looking at the % of marker positive cells (see FIGS. 3 A and B).

Similarly, the expression of NK cell ligands CD112, CD155 and ULBP3 and ULBP2/5/6, as well as ULBP1, was determined by flow cytometry on these early apoptotic (AnnexinV⁺/DAPI⁻) cell populations using ULBP-2/5/6 (Biocompare, USA), CD155 (Biolegend, USA), Nectin-2/CD112 (R&D Systems, USA), ULBP-1 (Biocompare, USA) and ULBP-3 antibody (Biocompare, USA). The early apoptotic cell population showed increasing expression of NK cell ligands CD112, CD155, ULBP3 and ULBP2/5/6, whereas a maximum had already been reached at 7 μg/kg for CD155 and 8 μg/kg for ULBP3 and ULBP2/5/6. The NK cell independent ligand ULBP1 did not show a significant change upon Kadcyla treatment. (see FIG. 4).

In summary the data show that Kadcyla mediated cell killing led to a large fraction of tumor cells undergoing ICD even in a tumor cell line with low to intermediate Her-2 expression.

### Example 3—PBMC Isolation and RLI-15 Treatment

In order to mimic the combined administration of Kadcyla and RLI-15 in a sequential schedule, human (PBMCs) from 3 donors were isolated from fresh human blood using Ficoll-Paque gradient and subsequently incubated for 72 h in the presence of 2.5 ng/ml of RLI-15.

### Example 4—In Vitro Activation of NK Cells by Tumor Cells Treated with Kadcyla

After the incubation period, dying tumor cells prepared in Example with increasing concentrations of Kadcyla were washed and transferred into fresh culture medium and added at 1:10 ratio (30,000 of tumor cells:300,000 of PBMC) to RLI-15 treated PBMCs prepared in Example and Example. These mixed cell populations were incubated for additional 4 h and the % of CD3⁻CD56⁺ cells (NK cells) was analyzed by flow cytometry using for markers CD3, CD56, CD107a and IFNγ (FIG. 5A). As controls non-treated tumor cells were incubated with non-treated PBMC (KadNT in the ADC group) and non-treated tumor cells were incubated with RLI-15 treated PBMC (KadNT in the RLI+ADC group).

The treatment of the tumor cells with Kadcyla led to no subsequent proliferation of (CD3-CD56⁺) NK cells upon co-cultivation with untreated PBMCs. On the other hand, RLI-15 alone led to an expected strong proliferation of NK cell, which was to some extent weaker if RLI-15 incubated PBMCs were co-cultivated with Kadcyla-treated tumor cells (FIG. 5B).

Looking at activated NK cells by plotting the % of CD107a⁺ NK cells of all NK cells (including both populations of CD107a⁺ and CD107a⁻ cells)—CD107a being an activation marker for NK cell, both the incubation of PBMCs with RLI-15 alone (RLI-15) or with Kadcyla-treated tumor cells (ADC groups) only led to a moderate activation of NK cells of up to 20% (compared to PBMC CTR), whereas the combination of RLI-15 treated PBMC with Kadcyla-treated tumor cells led to a strong activation of NK cells reaching a plateau of about 70% for 7-8 μg/ml Kadcyla (FIG. 5C).

The treatment of PBMCs with RLI-15 was sufficient for NK activation (see KadNT group), but the effect was considerably higher in combination with Kadcyla treated tumor cells (RLI+ADC Kad 5-10 μg/ml groups), where an increase of CD107a⁺ cells from <40% (KadNT) to about 70% (Kad 7 μg/ml and Kad 8 μg/ml) was observed. Also the increasing number of CD107a⁺ cells corresponded to the viability of the tumor cells measured by increasing AnnexinV⁺/DAPI⁻ cells (see FIG. 2B,C).

A very similar picture was seen when looking at the % of IFNγ⁺ NK cells as another measure of NK cell activation (FIG. 5D), again showing only moderate IFNγ producing NK cells for the Kadcyla only groups (up to about 10%) and the RLI-15 only group, whereas the combination of the RLI-15 incubated PBMCs with the Kadcyla-treated tumor cells lead to up to about 40% of IFNγ-producing NK cells peaking at 7-8 μg/ml Kadcyla.

In summary, we have shown that RLI-15 as single agent is able to significantly stimulate proliferation of NK cells (up to ˜40% compared to control PBMCs), whereas after co-incubation with Kadcyla pre-treated tumors cells we observed dramatic increase (up to 70% of CD3⁻CD56⁺CD107⁺ and up to 40% of CD3⁻CD56⁺IFNγ⁺-cells compared to RLI-15 only treated PBMCs) in activation of NK cells compared to those treated only with RLI-15 showing a strong synergy in vitro between induction of ICD mediated by Kadcyla and the immune-stimulatory effect of RLI-15, considered to have a predictive value of an in vivo efficacy.

Interestingly, we have observed the activation of NK cells already peaked at about 7 to 8 μg/ml Kadcyla in this in vitro setting. Such 7.7 μg/ml would be equivalent to about 0.5 mg/kg dose of Kadcyla in mice, which is much lower than typically applied doses of 15 mg/kg. Although this finding is difficult to transfer to an in vivo or even human situation, it still promises that such additional therapeutic effect of co-treatment with an IL-2/IL-15Rα agonist leads to increased efficacy of the ICD inducing agent at a lower dose, thereby increasing the therapeutic window.

### Example 5—Testing of Other ICD Inducing Agents or Modalities

Similar settings of the in vitro experiments described in Example 2 and Example 4 were used to screen for synergy to other ICD-inducing agents/modalities (selected standard of care chemotherapies “SoC”: doxorubicin or cisplatin).

Looking at other ADCs, a tumor cell line is required that expresses the respective target the antibody is directed to. For cytotoxic small molecules such as of anthracyclines, microtubule-destabilizing agents (Diederich 2019) (vinca alkaloids, taxanes such as paclitaxel, epothilone, eribulin, auristatin E, maytansine-derivatives), bleomycin, bortezomib, cyclophosphamide, platinum complexes (oxaliplatin, cisplatin) and nucleoside analogues, customary cell lines showing sensitivity to such drugs should be used. Suitable conditions for bortezomib have been described in Spisek et al. (2007).

This setting may also be used for ICD inducing treatment modalities such as high hydrostatic pressure (HHP), X-ray, γ or UV radiation, photodynamic therapy or hyperthermia/thermotherapy, where sensitive tumor cells are subjected to such physical stress under conditions inducing ICD, before being co-cultivated with the pre-treated PBMCs. Suitable conditions for inducing ICD physical stress are described for example in in WO 2013/004708, Adkins et al. (2014), and Adkins et al. (2017).

Clearly, RLI-15 can be replaced by other IL-2/IL-15βγ agonists known in the art in order to pre-treat PBMCs.

For the selected SoC, we have defined an optimal concentration and treatment duration (Table 2) for the efficient induction of ICD in a similar fashion as performed previously for Kadcyla analyzing cell death and cell surface exposure of ICD markers (FIG. 6, panel A, B). Doxorubicin and cisplatin were equally (cisplatin) or even better suitable to induce early apoptosis in the AGS tumor cell line (see FIG. 6A). Whereas looking at % of calreticulin, HSP70 and HSP90 positive cells, Kadcyla was more efficient compared to cisplatin and doxorubicin, with respect to the measure of mean fluorescent intensity (MFI), cisplatin appeared to have stronger induction and doxorubicin about equal (potentially stronger for calreticulin and HSP70 but weaker for HSP90), which may be interpreted that the chemotherapies induce relatively less cells to go into ICD but those induced show stronger ICD marker expression.

Subsequently, we have assessed the synergistic potential of doxorubicin and cisplatin to RLI-15 as measured by NK cell proliferation (CD3-CD56⁺ cell count), activation (CD107a release) and cytotoxicity (IFNγ). FIG. 7, panel A-C is a comparison of this data to data previously collected for Kadcyla (FIG. 5B-D). RLI-15 treatment led to an increase of total number of CD3⁻CD56⁺ cells (Panel A). Combination to SoC or Kadcyla led also to massive activation of these cells as shown by release of CD107a (Panel B) with high cytotoxic potential as shown by analysis of IFNγ secretion (Panel C) compared to PBMCs treated with RLI-15 only. Moreover, Kadcyla showed higher synergistic potential to RLI-15 in vitro than either doxorubicin or cisplatin as mainly represented by higher cytotoxic potential of NK cells after treatment with Kadcyla (Panel C). Specifically, the activation status and also the cytotoxic potential of NK cell after treatment with Kadcyla was significantly higher than that of NK cells after treatment with SoC (FIG. 7, panel C). This may be due to the antibody-dependent cellular toxicity (ADCC) activity of Trastuzumab in Kadcyla that may aside of the payload (DM1) may contribute to the synergistic effect in combination to RLI-15, as ADCC has been described as being synergistic as such to the activity of IL-2/IL-15βγ agonists.

### Example 6—Orthotopic huHER2/EMT-6 Breast Cancer Model In Vivo

The combination of Kadcyla and RLI-15 was tested in an orthotopic huHER2/EMT-6 breast cancer model in Balb/c AnN immunocompetent mice in vivo. The study was initiated when the initial mean tumor volume among individual groups reached 140 mm³. Kadcyla was dosed twice on study day 0 and 7 at a human equivalent dose (15 mg/kg) to potentially induce ICD prior to the RLI-15 treatment. RLI-15 was administered in 4 sequential doses on study days 15-18 to amplify the numbers and activate immune cells. The antitumor efficacy was evaluated on the level of absolute tumor volume change. Safety has been monitored by body weight loss in individual animals. HER2 expression in individual tumors was analyzed using HercepTest™ (Dako) at the endpoint to map for potential heterogeneity of the model (staining was performed according to the instructions given by the manufacturer).

Three spare study animals were sacrificed prior to the study start to collect tumors for the ex vivo analysis of HER2 expression by flow cytometry. This analysis revealed that approximately 90% of huHER2/EMT-6 tumor cells were positive for HER2 ex vivo (FIG. 8A).

For all residual tumors that had been collected at the end of the study and analyzed by IHC for HER2 expression it has been shown that the tumor expression level can be considered in vivo as rather intermediate with a mean H-score between 91,66-121.10 (see Table 4).

TABLE 4
Mean tumor HER2 expression represented by H-score per
group at study endpoint. The H score has been calculated
according to a following formula: H-Score = (% at 0) ×
0 + (% at 1) × 1 + (% at 2) × 2 + (% at 3) × 3 (0 ≤
H-score ≤ 300). Mean and standard deviation (SD) are shown.

| | Mean H-score per group | SD |
|---|---|---|
| G1 | 109.00 | 12.62 |
| G2 | 111.60 | 9.29 |
| G4 | 91.66 | 10.58 |
| G7 | 121.10 | 15.77 |

In the course of the study we have observed very homogeneous tumor growth among individual groups with a synergistic effect of the combination of RLI-15 and Kadcyla compared to single agent activities of both compounds (FIG. 8B). Whereas both RLI-15 single agent treatment at 1 mg/kg s.c. at days 15 to 18 and Kadcyla single agent treatment at 15 mg/kg i.v. at days 0 and 7 lead to an intermediate tumor volume increase, the combined treatment of these treatments lead to significantly reduced tumor volume at the end of the study.

Single animal data (FIG. 8C) showed that single agent activity of RLI-15 resulted in the complete tumor eradication in 2 out of 8 mice, while Kadcyla therapy alone led to 3 complete and durable responses and to a partial response with tumor relapse in another 3 animals. Such a tumor relapse is also frequently observed in patients and represents one of major liabilities of Kadcyla antitumor therapy. We have shown however that such relapse can be prevented when combining Kadcyla to RLI-15. This combination resulted in a durable and complete tumor eradication in 6 out of 8 animals indicating possible therapeutic perspective for this combination that might be used in common praxis.

In summary, the combination of RLI-15 and Kadcyla showed synergy in the antitumor efficacy in immune-resistant huHER2/EMT-6 tumors in vivo.

### Example 7—Cell Killing Assay In Vitro

The combination of the anti-CLDN18.2 directed ADC SOT102 in combination with SOT101 (RLI-15) was assessed in a cell killing assay in vitro. SOT102 is an antibody-drug-conjugate based on the anti-CLDN18.2 antibody hCl1a (SEQ ID NO: 20, SEQ ID NO: 21) having the ADCC inactivating heavy chain substitutions LALA (L234A|L235A) with the anthracycline PNU-159682 (PNU) linked to the C-terminus of the light chains by the non-cleavable linker GGGGSLPQTGG (SEQ ID NO: 24)-ethylenediamine (hCl1a-LC-G2-PNU) (SEQ ID NO: 22, SEQ ID NO: 23) as further described in Example 7 of WO 2022/136642.

Cell lines. Human A549 cells overexpressing CLDN18.2 (A549-CLDN18.2) were grown in DMEM medium (Gibco) supplemented with 10% fetal bovine serum, 2 mM glutamine (GlutaMAX, Gibco), 100 U/ml penicillin, 0.1 mg/ml streptomycin (Invitrogen) and 2 μg/ml puromycin (Gibco). Cells were maintained at 37° C. in a humidified atmosphere containing 5% CO₂.

Isolation of human NK cells: First, donors' blood (buffy coats, app. 70 ml of blood) was processed via ficoll density gradient centrifugation. Peripheral blood mononuclear cells (PBMCs) were collected, and human NK cells (hNK) were isolated using EasySep Human NK Cell Isolation Kit (STEMCELL) according to manufacturer's protocol. NK cells were washed and directly used into the assay. Purity of NK cell fraction was assessed via flow cytometry and reached over 70%.

Cell Killing Assay: A549_CLDN18.2 cells were seeded into 96-well plates (20.000 cells/well) and incubated overnight. Freshly isolated human NK (hNK) cells were resuspended in assay medium—RPMI 1640 (no phenol red) supplemented with 2 mM glutamine and 10% heat-inactivated (56° C. for 20 min) pooled complement human serum (Innovative Research). The medium from a 96-well plate containing adhered cells was aspirated and target cells were mixed with hNK cells to reach an E:T ratio of 10. Tested proteins were added at a concentration range of 0-100 μg/ml, SOT101 was added into appropriate wells to reach a 0.1 nM concentration. The mixture was incubated for 24 h at 37° C. and then cytotoxicity was measured as an activity of lactate dehydrogenase enzyme released from dead cells using the LDH Cytotoxicity Assay (Abcam, ab65393) according to manufacturer's protocol—10 μl of supernatant were transferred into a new 96-well plate, mixed with an LDH substrate and developed color change was measured using a spectrophotometer. Cytotoxicity was calculated as a percentage of the signal obtained from wells, where all seeded cells were permeabilized with lysis buffer (100% cytotoxicity).

Flow Cytometry: CLDN18.2 expression levels were measured via flow cytometry (BD LSRFortessa). Cells were collected by trypsinization, washed and labeled with a human primary anti-CLDN18.2 antibody (2 μg/ml) for 30 min at 4° C., followed by labeling with a goat anti-human secondary antibody conjugated with phycoerythrin (PE; eBiosciences, 12-4998-82) and DAPI to detect dead cells. For a negative control, cells were labeled with a secondary antibody and DAPI only.

Purity of isolated hNK cells was measured by staining the NK fraction with a set of fluorescently labeled antibodies to distinguish immune cell populations: anti-CD3 (APC-ef780, Thermo-Fisher Scientific), anti-CD16 (PE-Cy7, Biolegend), anti-CD56 (A700, Biolegend), anti-CD11c (APC, Exbio), Zombie Aqua Viability Dye (BV510, Biolegend). NK cells were gated as live CD3-CD11c⁻CD16⁺CD56⁺ cells. All obtained flow cytometry data were analyzed in FlowJo Software.

The anti-CLDN18.2 antibody as such (hCl1a WT) capable of ADCC was showing minor cytotoxic activity on the target cells under the tested conditions using freshly isolated NK cells, which was only insignificantly improved by the addition of SOT101. The ADC SOT102 alone, comprising the same CDRs as hCl1a WT but having the LALA substitutions minimizing the ADCC activity of the antibody, showed some cell killing, thus mediated by the linked PNU toxin. The combination of SOT102 with SOT101 then exerted a significantly higher cell killing activity (see FIG. 9). As the combination of hCl1a WT with SOT101 was not showing a significant difference, such increase is not mediated by ADCC (which is even further reduced in SOT102), i.e., the effect must be attributed to the combination of the NK cell activation by SOT101 with the ICD inducing toxin linked to the very much inert antibody. Also, given the absence of T cells and dendritic cells in this assay, this observed synergy is based on the direct effect of the delivered anthracycline on the tumor cells in interplay with the SOT101 stimulated NK cells. In other words, the anti-CLDN18.2 ADC SOT102 and SOT101 synergize in the killing of CLDN18.2 expressing target tumor cells in the presence of freshly isolated human NK cells, and such synergy is not based on ADCC or tumor antigen presentation by e.g. dendritic cells to cytotoxic T cells, and therefore can be attributed to ICD induced by the anthracycline PNU.

### Example 8—SOT101 and Oxaliplain Anti-Tumor Efficacy in MC38 Colon Carcinoma Model In Vivo

C57BL/6 mice were injected s.c. with 5×10⁵ MC38 colon carcinoma cells. Starting from day 3 after tumor cell inoculation, mice were treated i.p. with 7.5 mg/kg oxaliplatin Q2W or s.c. with 2×2 mg/kg SOT101 on W1 and W2 or with combination of both, according to combination schedules 1 (oxaliplatin 7.5 mg/kg i.p. D3 and D17+SOT101 s.c. 2×2 mg/kg D4,5 and D18,19), 2 (oxaliplatin 7.5 mg/kg i.p. D3 and D17+SOT101 s.c. 2×2 mg/kg D4,5 and D11,12) and schedule 3 (oxaliplatin 7.5 mg/kg i.p. D3+SOT101 s.c. 2×2 mg/kg D4,5 and D11,12). Individual mouse weight and tumor growth was monitored. On day 21, mice were euthanized. The combined SOT101 and oxaliplatin treatment was well tolerated given no significant difference in relative mouse body weight (data not shown). Results indicate increased efficacy for combined administrations (data not shown).

| Sequences | | |
|---|---|---|
| human IL-2 | | |
| SEQ ID NO: 1 | | |
| 1 | MYRMQLLSCI ALSLALVINS APTSSSTKKT QLQLEHLLLD LQMILNGINN YKNPKLTRML | |
| 61 | TFKFYMPKKA TELKHLQCLE EELKPLEEVL NLAQSKNFHL RPRDLISNIN VIVLELKGSE | |
| 121 | TTFMCEYADE TATIVEFLNR WITFCQSIIS TLT | 153 |
| mature human IL-2 | | |
| SEQ ID NO: 2 | | |
| 1 | APTSSSTKKT QLQLEHLLLD LOMILNGINN YKNPKLTRML TFKFYMPKKA TELKHLQCLE | |
| 61 | EELKPLEEVL NLAQSKNFHL RPRDLISNIN VIVLELKGSE TTFMCEYADE TATIVEFLNR | |
| 121 | WITFCQSIIS TLT | 133 |
| human IL-15 | | |
| SEQ ID NO: 3 | | |
| 1 | MRISKPHLRS ISIQCYLCLL LNSHFLTEAG IHVFILGCES AGLPKTEANW VNVISDLKKI | |
| 61 | EDLIQSMHID ATLYTESDVH PSCKVTAMKC FLLELQVISL ESGDASIHDT VENLIILANN | |
| 121 | SLSSNGNVTE SGCKECEELE EKNIKEFLQS FVHIVOMFIN TS | 162 |
| mature human IL-15 | | |
| SEQ ID NO: 4 | | |
| 1 | NWVNVISDLK KIEDLIQSMH IDATLYTESD VHPSCKVTAM KCFLLELQVI SLESGDASIH | |
| 61 | DTVENLIILA NNSLSSNGNV TESGCKECEE LEEKNIKEFL QSFVHIVOMF INTS | 114 |
| human IL-15Rα | | |
| SEQ ID NO: 5 | | |
| 1 | MAPRRARGCR TLGLPALLLL LLLRPPATRG ITCPPPMSVE HADIWVKSYS LYSRERYICN | |
| 61 | SGFKRKAGTS SLTECVLNKA TNVAHWTTPS LKCIRDPALV HQRPAPPSTV TTAGVTPQPE | |
| 121 | SLSPSGKEPA ASSPSSNNTA ATTAAIVPGS QLMPSKSPST GTTEISSHES SHGTPSQTTA | |
| 181 | KNWELTASAS HQPPGVYPQG HSDTTVAIST STVLLCGLSA VSLLACYLKS RQTPPLASVE | |
| 241 | MEAMEALPVT WGTSSRDEDL ENCSHHL | 267 |
| human sushi domain of IL-15Rα | | |
| SEQ ID NO: 6 | | |
| 1 | CPPPMSVEHA DIWVKSYSLY SRERYICNSG FKRKAGTSSL TECVLNKATN VAHWTTPSLK | |
| | C | 61 |
| sushi + fragment of IL-15Rα | | |
| SEQ ID NO: 7 | | |
| 1 | ITCPPPMSVE HADIWVKSYS LYSRERYICN SGFKRKAGTS SLTECVLNKA TNVAHWTTPS | |
| 61 | LKCIRDPALV HORPAPP | 77 |
| linker | | |
| SEQ ID NO: 8 | | |
| 1 | SGGSGGGGSG GGSGGGGSGG | 20 |
| RLI2 | | |
| SEQ ID NO: 9 | | |
| 1 | ITCPPPMSVE HADIWVKSYS LYSRERYICN SGFKRKAGTS SLTECVLNKA TNVAHWTTPS | |
| 61 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNGNVTES | |
| 181 | GCKECEELEE KNIKEFLOSF VHIVOMFINT S | 211 |
| IL2v | | |
| SEQ ID NO: 10 | | |
| 1 | APASSSTKKT QLQLEHLLLD LOMILNGINN YKNPKLTRML TAKFAMPKKA TELKHLQCLE | |
| 61 | EELKPLEEVL NGAQSKNFHL RPRDLISNIN VIVLELKGSE TTFMCEYADE TATIVEFLNR | |
| 121 | ITFAQSIIS TLT | 132 |
| Leader peptide of (IL-15_{N72D})₂:IL-15Rαₛᵤₛₕᵢ-FC: | | |
| SEQ ID NO: 11 | | |
| 1 | METDTLLLWV LLLWVPGSTG | 20 |
| IL-15Rαₛᵤₛₕᵢ (65aa)-Fc (IgG1 CH2-CH3): | | |
| SEQ ID NO: 12 | | |
| 1 | ITCPPPMSVE HADIWVKSYS LYSRERYICN SGFKRKAGTS SLTECVLNKA TNVAHWTTPS | |
| 61 | LKCIREPKSC DKTHTCPPCP APELLGGPSV FLFPPKPKDT LMISRTPEVT CVVVDVSHED | |
| 121 | PEVKFNWYVD GVEVHNAKTK PREEQYNSTY RVVSVLTVLH QDWLNGKEYK CKVSNKALPA | |
| 181 | PIEKTISKAK GQPREPQVYT LPPSRDELTK NQVSLTCLVK GFYPSDIAVE WESNGQPENN | |
| 241 | YKTTPPVLDS DGSFFLYSKL TVDKSRWQQG NVFSCSVMHE ALHNHYTOKS LSLSPGK | 297 |
| IL-15_{N72D} | | |
| SEQ ID NO: 13 | | |
| 1 | NWVNVISDLK KIEDLIQSMH IDATLYTESD VHPSCKVTAM KCFLLELQVI SLESGDASIH | |
| 61 | DTVENLIILA NDSLSSNGNV TESGCKECEE LEEKNIKEFL QSFVHIVOMF INTS | 114 |
| hCl1a HCDR1 | | |
| SEQ ID NO: 14 | | |
| DYAMH | | |
| hCl1a HCDR2 | | |
| SEQ ID NO: 15 | | |
| WINTYTGKPTYAQKFQG | | |
| hCl1a HCDR3 | | |
| SEQ ID NO: 16 | | |
| AVFYGYTMDA | | |
| hCl1a LCDR1 | | |
| SEQ ID NO: 17 | | |
| RASEDIYSNLA | | |
| hCl1a LCDR2 | | |
| SEQ ID NO: 18 | | |
| SVKRLQD | | |
| hCl1a LCDR3 | | |
| SEQ ID NO: 19 | | |
| LQGSNFPLT | | |
| hCl1a HC | | |
| SEQ ID NO: 20 | | |
| QVQLVQSGAEVKKPGASVKVSCKASGYTFTDYAMHWVRQAPGORLEWMGWINTYTGKPTYAQKFQGRVT | | |
| ITRDTSASTAYMELSSLRSEDTAVYYCARAVFYGYTMDAWGQGTLVTVSSASTKGPSVFPLAPSSKSTS | | |
| GGTAALGCLVKDYFPEPVTVSWNSGALTSGVHTFPAVLQSSGLYSLSSVVTVPSSSLGTQTYICNVNHK | | |
| PSNTKVDKKVEPKSCDKTHTCPPCPAPELLGGPSVFLFPPKPKDTLMISRTPEVTCVVVDVSHEDPEVK | | |
| FNWYVDGVEVHNAKTKPREEQYNSTYRVVSVLTVLHQDWLNGKEYKCKVSNKALPAPIEKTISKAKGQP | | |
| REPQVYTLPPSRDELTKNQVSLTCLVKGFYPSDIAVEWESNGQPENNYKTTPPVLDSDGSFFLYSKLTV | | |
| DKSRWQQGNVFSCSVMHEALHNHYTQKSLSLSPGK | | |
| hCl1a LC | | |
| SEQ ID NO: 21 | | |
| DIQMTQSPSSLSASVGDRVTITCRASEDIYSNLAWYQQKPGKAPKLLIFSVKRLQDGVPSRFSGSGSGT | | |
| DFTLTISSLOPEDFATYYCLQGSNFPLTFGQGTKVEIKRTVAAPSVFIFPPSDEQLKSGTASVVCLLNN | | |
| FYPREAKVQWKVDNALQSGNSQESVTEQDSKDSTYSLSSTLTLSKADYEKHKVYACEVTHQGLSSPVTK | | |
| SFNRGEC | | |
| hCl1a HC LALA | | |
| SEQ ID NO: 22 | | |
| 01 | QVQLVQSGAE VKKPGASVKV SCKASGYTFT DYAMHWVRQA PGQRLEWMGW | 50 |
| 51 | INTYTGKPTY AQKFQGRVTI TRDTSASTAY MELSSLRSED TAVYYCARAV | 100 |
| 101 | FYGYTMDAWG QGTLVTVSSA STKGPSVFPL APSSKSTSGG TAALGCLVKD | 150 |
| 151 | YFPEPVTVSW NSGALTSGVH TFPAVLQSSG LYSLSSVVTV PSSSLGTQTY | 200 |
| 201 | ICNVNHKPSN TKVDKKVEPK SCDKTHTCPP CPAPEAAGGP SVFLFPPKPK | 250 |
| 251 | DTLMISRTPE VTCVVVDVSH EDPEVKENWY VDGVEVHNAK TKPREEQYNS | 300 |
| 301 | TYRVVSVLTV LHQDWLNGKE YKCKVSNKAL PAPIEKTISK AKGQPREPQV | 350 |
| 351 | YTLPPSRDEL TKNQVSLTCL VKGFYPSDIA VEWESNGOPE NNYKTTPPVL | 400 |
| 401 | DSDGSFFLYS KLTVDKSRWQ QGNVFSCSVM HEALHNHYTQ KSLSLSPGK | |
| hCl1a LC-sortase tag | | |
| SEQ ID NO: 23 | | |
| 01 | DIQMTQSPSS LSASVGDRVT ITCRASEDIY SNLAWYQQKP GKAPKLLIFS | 51 |
| 51 | VKRLODGVPS RFSGSGSGTD FTLTISSLOP EDFATYYCLQ GSNFPLTFGQ | 100 |
| 101 | GTKVEIKRTV AAPSVFIFPP SDEQLKSGTA SVVCLLNNFY PREAKVOWKV | 150 |
| 151 | DNALQSGNSQ ESVTEQDSKD STYSLSSTLT LSKADYEKHK VYACEVTHQG | 200 |
| 201 | LSSPVTKSEN RGECGGGGSL PQTGG | |

Sortase Linker
SEQ ID NO: 24
GGGGSLPQTGG

### REFERENCES

US2003/0124678
US2006/0057680
US2007/0036752
US2018/0118805
U.S. Pat. No. 5,229,109
U.S. Pat. No. 10,206,980
WO2005/085282A1
WO2006/020849A2
WO2007/059997
WO2008/003473A2
WO2008/143794A1
WO2009/135031A1
WO2012/107417A1
WO2013/004708A1
WO2014/140317A2
WO2014/145806A2
WO2014/207173A1
WO2014/066527
WO2015/097037
WO2016/060996A2
WO2016/102679A1
WO2016/142314A1
WO2016/165762
WO2016/166122
WO2017/046200A1
WO2017/112528A2
WO2018/071918A1
WO2018/071919A1
WO2018/102536
WO2018/151868
WO2018/213341A1
WO2018/234862
WO2019/028419A1
WO2019/110725A1
WO2019/113221
WO2019/145469
WO2019/145471
WO2019/165453A1
WO2019/166946A1
WO2019/246379A1
WO2019/7185705
WO2020/069398
WO2020/232305
WO2021/111003A1
WO2020/234387A1
WO2021/130291A1
WO 2021/7245130
WO2022/136642A1
Adkins, I., et al. (2014). “Physical modalities inducing immunogenic tumor cell death for cancer immunotherapy.” Oncoimmunology 3 (12): e968434.
Adkins, I., et al. (2018). “High hydrostatic pressure in cancer immunotherapy and biomedicine.” Biotechnol Adv.
Adkins, I., et al. (2017). “Severe, but not mild heat-shock treatment induces immunogenic cell death in cancer cells.” OncoImmunology 6 (5): e1311433.
Antosova, Z., et al. (2020). “562 SO-C101 induces NK cell cytotoxicity and potentiates antibody-dependent cell cytotoxicity and anti-tumor activity.” Journal for ImmunoTherapy of Cancer 8 (Suppl 3): A339-A339.
Arneja, A., et al. (2014). “Qualitatively different T cell phenotypic responses to IL-2 versus IL-15 are unified by identical dependences on receptor signal strength and duration.” J Immunol 192(1): 123-135.
Bacac, M., et al. (2017). “Abstract 1594: Enhancement of the anti-tumor activity of CEA TCB via combination with checkpoint blockade by PD-L1 and interleukin-2 variant immunocytokine.” Cancer Research 77 (13 Supplement): 1594.
Bacac, M., et al. (2016). “A Novel Carcinoembryonic Antigen T-Cell Bispecific Antibody (CEA TCB) for the Treatment of Solid Tumors.” Clin Cancer Res 22(13): 3286-3297.
Bauzon, M., et al. (2019). “Maytansine-bearing antibody-drug conjugates induce in vitro hallmarks of immunogenic cell death selectively in antigen-positive target cells.” Oncoimmunology 8 (4): e1565859.
Charych, D., et al. (2017). “Modeling the receptor pharmacology, pharmacokinetics, and pharmacodynamics of NKTR-214, a kinetically-controlled interleukin-2 (IL2) receptor agonist for cancer immunotherapy.” PLoS One 12(7): e0179431.
Charych, D. H., et al. (2016). “NKTR-214, an Engineered Cytokine with Biased IL2 Receptor Binding, Increased Tumor Exposure, and Marked Efficacy in Mouse Tumor Models.” Clinical Cancer Research 22(3): 680.
Chenoweth, M. J., et al. (2012). “IL-15 can signal via IL-15Ralpha, JNK, and NF-kappaB to drive RANTES production by myeloid cells.” J Immunol 188(9): 4149-4157.
Conlon, K., et al. (2019). Phase I/Ib study of NIZ985 with and without spartalizumab (PDR001) in patients with metastatic/unresectable solid tumors. AACR Annual Meeting, Atlanta, GA.
Conlon, K. C., et al. (2019). “Cytokines in the Treatment of Cancer.” J Interferon Cytokine Res 39(1): 6-21.
D'Amico, L., et al. (2019). “A novel anti-HER2 anthracycline-based antibody-drug conjugate induces adaptive anti-tumor immunity and potentiates PD-1 blockade in breast cancer.” J Immunother Cancer 7(1): 16.
Darvin, P., et al. (2018). “Immune checkpoint inhibitors: recent progress and potential biomarkers.” Exp Mol Med 50(12): 165.
De Sousa Linhares, A., et al. (2018). “Not All Immune Checkpoints Are Created Equal.” Frontiers in Immunology 9 (1909).
Diederich, M. (2019). “Natural compound inducers of immunogenic cell death.” Arch Pharm Res 42(7): 629-645.
Dudek, A. M., et al. (2013). “Inducers of immunogenic cancer cell death.” Cytokine Growth Factor Rev 24(4): 319-333.
Dumontet, C. and M. A. Jordan (2010). “Microtubule-binding agents: a dynamic field of cancer therapeutics.” Nature reviews. Drug discovery 9(10): 790-803.
Edgar, R. C. (2004). “MUSCLE: multiple sequence alignment with high accuracy and high throughput.” Nucleic Acids Res 32(5): 1792-1797.
Friedman, M. and S. Stahl (2009). “Engineered affinity proteins for tumour-targeting applications.” Biotechnol Appl Biochem 53 (Pt 1): 1-29.
Fucikova, J., et al. (2011). “Human tumor cells killed by anthracyclines induce a tumor-specific immune response.” Cancer Res 71(14): 4821-4833.
Fucikova, J., et al. (2014). “High hydrostatic pressure induces immunogenic cell death in human tumor cells.” Int J Cancer 135(5): 1165-1177.
Gajewski, T. F., et al. (2013). “Cancer immunotherapy strategies based on overcoming barriers within the tumor microenvironment.” Curr Opin Immunol 25(2): 268-276.
Gearing, A. J. and R. Thorpe (1988). “The international standard for human interleukin-2. Calibration by international collaborative study.” J Immunol Methods 114(1-2): 3-9.
Gerber, H. P., et al. (2016). “Combining antibody-drug conjugates and immune-mediated cancer therapy: What to expect?” Biochem Pharmacol 102: 1-6.
Giron-Michel, J., et al. (2005). “Membrane-bound and soluble IL-15/IL-15Ralpha complexes display differential signaling and functions on human hematopoietic progenitors.” Blood 106(7): 2302-2310.
Giroux, S. (2013). “Overcoming acquired resistance to kinase inhibition: the cases of EGFR, ALK and BRAF.” Bioorg Med Chem Lett 23(2): 394-401.
Godar, M., et al. (2018). “Therapeutic bispecific antibody formats: a patent applications review (1994-2017).” Expert Opinion on Therapeutic Patents 28(3): 251-276.
Golden, E. B. and L. Apetoh (2015). “Radiotherapy and Immunogenic Cell Death.” Seminars in Radiation Oncology 25(1): 11-17.
Goujon, M., et al. (2010). “A new bioinformatics analysis tools framework at EMBL-EBI.” Nucleic Acids Res 38 (Web Server issue): W695-699.
Haratani, K., et al. (2020). “U3-1402 sensitizes HER3-expressing tumors to PD-1 blockade by immune activation.” J Clin Invest 130(1): 374-388.
Hayashi, K., et al. (2021). “Inhibitory DAMPs in immunogenic cell death and its clinical implications.” Cell Stress 5(4): 52-54.
Heaton, K. M., et al. (1993). “Human interleukin 2 analogues that preferentially bind the intermediate-affinity interleukin 2 receptor lead to reduced secondary cytokine secretion: implications for the use of these interleukin 2 analogues in cancer immunotherapy.” Cancer Res 53(11): 2597-2602.
Holliger, P. and P. J. Hudson (2005). “Engineered antibody fragments and the rise of single domains.” Nat Biotechnol 23(9): 1126-1136.
Hori, T., et al. (1987). “Establishment of an interleukin 2-dependent human T cell line from a patient with T cell chronic lymphocytic leukemia who is not infected with human T cell leukemia/lymphoma virus.” Blood 70(4): 1069-1072.
Hu, P., et al. (2003). “Generation of low-toxicity interleukin-2 fusion proteins devoid of vasopermeability activity.” Blood 101(12): 4853-4861.
Hu, Q., et al. (2018). “Discovery of a novel IL-15 based protein with improved developability and efficacy for cancer immunotherapy.” Sci Rep 8(1): 7675.
Iwata, T. N., et al. (2018). “A HER2-Targeting Antibody-Drug Conjugate, Trastuzumab Deruxtecan (DS-8201a), Enhances Antitumor Immunity in a Mouse Model.” Mol Cancer Ther 17(7): 1494-1503.
Jain, N., et al. (2015). “Current ADC Linker Chemistry.” Pharm Res 32(11): 3526-3540.
Joseph, I. B., et al. (2019). “THOR-707, a novel not-alpha IL-2, elicits durable pharmacodynamic responses in non-human primates and efficacy as single agent and in combination with anti PD-1 in multiple syngeneic mouse models.” Proceedings of the American Association for Cancer Research 60: 838.
Khongorzul, P., et al. (2020). “Antibody-Drug Conjugates: A Comprehensive Review.” Molecular Cancer Research 18(1): 3.
Kitai, Y., et al. (2017). “DNA-Containing Exosomes Derived from Cancer Cells Treated with Topotecan Activate a STING-Dependent Pathway and Reinforce Antitumor Immunity.” J Immunol 198(4): 1649-1659.
Klein, C. (2014). “S41. Novel CEA-targeted IL2 variant immunocytokine for immunotherapy of cancer.” Journal for Immunotherapy of Cancer 2 (Suppl 2): I8-I8.
Klein, C., et al. (2013). “Abstract PR8: Novel tumor-targeted, engineered IL-2 variant (IL-2v)-based immunocytokines for immunotherapy of cancer.” Cancer Research 73 (1 Supplement): PR8.
Klein, C., et al. (2017). “Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines.” Oncoimmunology 6 (3): e1277306.
Kroemer, G., et al. (2013). “Immunogenic cell death in cancer therapy.” Annu Rev Immunol 31: 51-72.
Kurowska, M., et al. (2002). “Fibroblast-like synoviocytes from rheumatoid arthritis patients express functional IL-15 receptor complex: endogenous IL-15 in autocrine fashion enhances cell proliferation and expression of Bcl-x(L) and Bcl-2.” J Immunol 169(4): 1760-1767.
Lopes, J. E., et al. (2020). “ALKS 4230: a novel engineered IL-2 fusion protein with an improved cellular selectivity profile for cancer immunotherapy.” J Immunother Cancer 8 (1).
LoRusso, P. M., et al. (2011). “Trastuzumab emtansine: a unique antibody-drug conjugate in development for human epidermal growth factor receptor 2-positive cancer.” Clin Cancer Res 17(20): 6437-6447.
Mahmood, I. (2021). “Clinical Pharmacology of Antibody-Drug Conjugates.” Antibodies (Basel, Switzerland) 10(2): 20.
Martin, A. C. R. and J. Allemn (2014). Bioinformatics Tools for Analysis of Antibodies. Handbook of Therapeutic Antibodies, Second Edition, Wiley-VCH Verlag GmbH & Co. KGaA.: 201-228.
McDermott, U. and J. Settleman (2009). “Personalized cancer therapy with selective kinase inhibitors: an emerging paradigm in medical oncology.” J Clin Oncol 27(33): 5650-5659.
Mendoza, J. L., et al. (2019). “Structure of the IFNgamma receptor complex guides design of biased agonists.” Nature 567(7746): 56-60.
Merchant, R., et al. (2022). “Fine-tuned long-acting interleukin-2 superkine potentiates durable immune responses in mice and non-human primate.” J Immunother Cancer 10 (1).
Minotti, G., et al. (2004). “Anthracyclines: molecular advances and pharmacologic developments in antitumor activity and cardiotoxicity.” Pharmacol Rev 56(2): 185-229.
Needleman, S. B. and C. D. Wunsch (1970). “A general method applicable to the search for similarities in the amino acid sequence of two proteins.” J Mol Biol 48(3): 443-453.
O'Neil, J., et al. (2021). “Tumor-selective activity of XTX202, a protein-engineered IL-2, in mice without peripheral toxicities in nonhuman primates.” Journal of Clinical Oncology 39 (15_suppl): 2563-2563.
Obeid, M., et al. (2007). “Calreticulin exposure is required for the immunogenicity of gamma-irradiation and UVC light-induced apoptosis.” Cell Death Differ 14(10): 1848-1850.
Panowski, S., et al. (2014). “Site-specific antibody drug conjugates for cancer therapy.” MAbs 6(1): 34-45.
Pearson, W. R. and D. J. Lipman (1988). “Improved tools for biological sequence comparison.” Proc Natl Acad Sci USA 85(8): 2444-2448.
Perdreau, H., et al. (2010). “Different dynamics of IL-15R activation following IL-15 cis- or trans-presentation.” Eur Cytokine Netw 21(4): 297-307.
Pol, J., et al. (2015). “Trial Watch: Immunogenic cell death inducers for anticancer chemotherapy.” Oncoimmunology 4 (4): e1008866.
Quintieri, L., et al. (2005). “Formation and antitumor activity of PNU-159682, a major metabolite of nemorubicin in human liver microsomes.” Clin Cancer Res 11(4): 1608-1617.
Ring, A. M., et al. (2012). “Mechanistic and structural insight into the functional dichotomy between IL-2 and IL-15.” Nat Immunol 13(12): 1187-1195.
Rios-Doria, J., et al. (2017). “Antibody-Drug Conjugates Bearing Pyrrolobenzodiazepine or Tubulysin Payloads Are Immunomodulatory and Synergize with Multiple Immunotherapies.” Cancer Res 77(10): 2686-2698.
Robinson, T. O. and K. S. Schluns (2017). “The potential and promise of IL-15 in immuno-oncogenic therapies.” Immunol Lett 190: 159-168.
Romee, R., et al. (2018). “First-in-human Phase 1 Clinical Study of the IL-15 Superagonist Complex ALT-803 to Treat Relapse after Transplantation.” Blood 131(23): 2515-2527.
Rosen, D. B., et al. (2022). “TransCon IL-2 β/γ: a novel long-acting prodrug with sustained release of an IL-2Rβ/γ-selective IL-2 variant with improved pharmacokinetics and potent activation of cytotoxic immune cells for the treatment of cancer.” J Immunother Cancer 10 (7).
Rosenzweig, S. A. (2012). “Acquired resistance to drugs targeting receptor tyrosine kinases.” Biochemical pharmacology 83(8): 1041-1048.
Santara, S. S., et al. (2021). “The NK receptor NKp46 recognizes ecto-calreticulin on ER-stressed cells.” bioRxiv: 2021.2010.2031.466654.
Shanafelt, A. B., et al. (2000). “A T-cell-selective interleukin 2 mutein exhibits potent antitumor activity and is well tolerated in vivo.” Nat Biotechnol 18(11): 1197-1202.
Silva, D.-A., et al. (2019). “De novo design of potent and selective mimics of IL-2 and IL-15.” Nature 565(7738): 186-191.
Silva, D. A. (2022). “An IL-2 Protein Therapeutic for Cancer that Gains Function on the Spot.” Cancer Immunol Res 10(5): 544.
Smith, T. F. and M. S. Waterman (1981). “Comparison of biosequences.” Advances in Applied Mathematics 2(4): 482-489.
Sockolosky, J. T., et al. (2018). “Selective targeting of engineered T cells using orthogonal IL-2 cytokine-receptor complexes.” Science 359(6379): 1037.
Solomon, B. L. and I. Garrido-Laguna (2018). “TIGIT: a novel immunotherapy target moving from bench to bedside.” Cancer Immunol Immunother 67(11): 1659-1667.
Soman, G., et al. (2009). “MTS dye based colorimetric CTLL-2 cell proliferation assay for product release and stability monitoring of interleukin-15: assay qualification, standardization and statistical analysis.” J Immunol Methods 348(1-2): 83-94.
Spisek, R., et al. (2007). “Bortezomib enhances dendritic cell (DC)-mediated induction of immunity to 50 human myeloma via exposure of cell surface heat shock protein 90 on dying tumor cells: therapeutic implications.” Blood 109(11): 4839-4845.
Steel, J. C., et al. (2012). “Interleukin-15 biology and its therapeutic implications in cancer.” Trends Pharmacol Sci 33(1): 35-41.
Tan, X., et al. (2018). “Abstract 2757: Antibody-drug conjugate payloads induce markers of immunogenic cell death in cancer cells.” Cancer Research 78 (13 Supplement): 2757.
Thaysen-Andersen, M., et al. (2016). “Recombinant human heterodimeric IL-15 complex displays extensive and reproducible N- and O-linked glycosylation.” Glycoconj J 33(3): 417-433.
Verma, S., et al. (2012). “Trastuzumab emtansine for HER2-positive advanced breast cancer.” N Engl J Med 367(19): 1783-1791.
Vlahov, I. R., et al. (2017). “Latent Warheads for Targeted Cancer Therapy: Design and Synthesis of pro-Pyrrolobenzodiazepines and Conjugates.” Bioconjug Chem 28(12): 2921-2931.
Wadhwa, M., et al. (2013). “The 2nd International standard for Interleukin-2 (IL-2) Report of a collaborative study.” Journal of Immunological Methods 397(1): 1-7.
Waldmann, T. A. (2015). “The shared and contrasting roles of IL2 and IL15 in the life and death of normal and neoplastic lymphocytes: implications for cancer therapy.” Cancer Immunol Res 3(3): 219-227.
Wei, X., et al. (2001). “The Sushi domain of soluble IL-15 receptor alpha is essential for binding IL-15 and inhibiting inflammatory and allogenic responses in vitro and in vivo.” J Immunol 167(1): 277-282.
Weinstein, J. N. (2005). Oncogenomics: Molecular Approaches to Cancer—Linking Drugs and Genes: Pharmacogenomics, Pharmacoproteomics, Bioinformatics, and the NCI-60, John Wiley & Sons, Inc.
Wrangle, J. M., et al. (2018). “ALT-803, an IL-15 superagonist, in combination with nivolumab in patients with metastatic non-small cell lung cancer: a non-randomised, open-label, phase 1b trial.” Lancet Oncol 19(5): 694-704.
Zhou, J., et al. (2019). “Immunogenic cell death in cancer therapy: Present and emerging inducers.” J Cell Mol Med 23(8): 4854-4865.
Zingoni, A., et al. (2017). “Natural Killer Cell Response to Chemotherapy-Stressed Cancer Cells: Role in Tumor Immunosurveillance.” Front Immunol 8: 1194.

1. An interleukin-2/interleukin-15 receptor βγ (IL-2/IL-15Rβγ) agonist for use in treating cancer in a patient, wherein said IL-2/IL-15Rβγ agonist,
a. is administered simultaneously with or sequentially to a cytotoxic compound capable of inducing immunogenic cell death (ICD),
b. is administered simultaneously with or sequentially to applying a modality capable of inducing ICD,
c. is administered simultaneously with a cytotoxic compound capable of inducing ICD and simultaneously with a modality capable of inducing ICD,
d. is administered simultaneously with a cytotoxic compound capable of inducing ICD and sequentially to a modality capable of inducing ICD,
e. is administered sequentially to a cytotoxic compound capable of inducing ICD and simultaneously with a modality capable of inducing ICD, or
f. is administered sequentially to a cytotoxic compound capable of inducing ICD and sequentially to a modality capable of inducing ICD.

2. The IL-2/IL-15Rβγ agonist for use of claim 1, wherein, in the case of sequential administration, said IL-2/IL-15Rβγ agonist is administered prior to and/or subsequent to said cytotoxic compound capable of inducing ICD, or prior to and/or subsequent to said modality capable of inducing ICD.

3. The IL-2/IL-15Rβγ agonist for use of claim 1 or claim 2, wherein, in the case of sequential administration, said IL-2/IL-15Rβγ agonist is administered subsequent to said cytotoxic compound capable of ICD or subsequent to said modality capable of inducing ICD.

4. The IL-2/IL-15Rβγ agonist for use of claim 1, wherein, in the case of simultaneous administration, said IL-2/IL-15Rβγ agonist and said cytotoxic compound capable of inducing ICD are provided as components of the same pharmaceutical compositions or as components of separate pharmaceutical compositions.

5. The IL-2/IL-15Rβγ agonist for use according to any of claims 1 to 4, wherein said cytotoxic compound capable of inducing ICD is selected from the group consisting of an anthracycline; a microtubule-destabilizing agent including a vinca alkaloid, a taxane, an epothilone, eribulin, an auristatin, and maytansine or a maytansinoid, and tubulysine; bleomycin; a proteasomal inhibitor including bortezomib; an alkylating agent including cyclophosphamide, a platinum complex including oxaliplatin, and a pyrrolo-benzodiazepine, calicheamicin derivatives and topoisomerase I inhibitors, and a nucleoside analogue.

6. The IL-2/IL-15Rβγ agonist for use according to any of claims 1 to 5, wherein said cytotoxic compound capable of inducing ICD is covalently linked to an antibody forming an antibody-drug conjugate (ADC).

7. The IL-2/IL-15Rβγ agonist for use according to claim 5 or 6, wherein said cytotoxic compound is an anthracycline, a maytansine or maytansinoid, a topoisomerase I inhibitor, or a calicheamicin derivative,
preferably wherein the topoisomerase I inhibitor is not SN38.

8. The IL-2/IL-15Rβγ agonist for use according to any of the claim 5 to 7,
wherein said anthracycline is selected from the group consisting of daunorubicin, doxorubicin, epirubicin, idarubicin, mitoxantrone and PNU-159682;
wherein said maytansine or maytansinoid is selected from the group consisting of maytansine, mertansine/emtansine (DM1), ansamitocin and ravtansine/soravtansine (DM4);
wherein said topoisomerase I inhibitor is a topotecan, exatecan or a exatecan derivative, especially DS-8201a or DX-8951; or
wherein said calicheamicin is Calicheamicin γ₁^{I}.

9. The IL-2/IL-15Rβγ agonist for use according to any of claims 6 to 8, wherein said antibody is an antibody which specifically binds to HER2, preferably trastuzumab; binds to HER3, preferably patritumab; binds to Nectin-4, preferably enfortumab; binds to CD33, preferably gemtuzumab or IMGN779, more preferably gemtuzumab; binds to CD30, preferably brentuximab; binds to CD22, preferably inotuzumab, or CD79B, preferably polatuzumab; binds to ROR-1, preferably NBE-002; or binds to CLDN18.2, preferably zolbetuximab or a humanized variant thereof.

10. The IL-2/IL-15Rβγ agonist for use according to any of claims 6 to 9, wherein the ADC is trastuzumab emtansine, trastuzumab deruxtecan, gemtuzumab ozogamicin, inotuzumab ozogamicin, brentuximab vedotin, enfortumab vedotin and polatuzumab vedotin, especially trastuzumab emtansine or enfortumab vedotin.

11. The IL-2/IL-15Rβγ agonist for use according to any of claims 1 to 10, wherein the patient is suffering from tumors expressing HER2, preferably wherein the patient has been diagnosed as having a tumor with low to mediate HER2 expression.

12. The IL-2/IL-15Rβγ agonist for use according to any of claims 1 to 3, wherein the modality capable of inducing ICD is selected from high hydrostatic pressure (HHP), photodynamic therapy, UV radiation, radiotherapy, oncolytic virus therapy and thermotherapy.

13. The IL-2/IL-15Rβγ agonist for use according to any of claims 1 to 12, wherein the IL-2/IL-15Rβγ agonist is an interleukin 15 (IL-15)/interleukin-15 receptor alpha (IL-15Rα) complex.

14. The IL-2/IL-15Rβγ agonist for use according to any of claims 1 to 13 wherein the IL-2/IL-15Rβγ agonist is an interleukin 15 (IL-15)/interleukin-15 receptor alpha (IL-15Rα) complex, wherein the complex is a fusion protein comprising the sushi domain of human IL-15Rα or a derivative thereof, a flexible linker and the human IL-15 or a derivative thereof
preferably wherein the human IL-15Rα sushi domain comprises the amino acid sequence of SEQ ID NO: 6, and wherein the human IL-15 comprises the amino acid sequence of SEQ ID NO: 4.

15. The IL-2/IL-15Rβγ agonist for use according to any of claims 1 to 14, wherein the IL-2/IL-15Rβγ agonist is an interleukin 15 (IL-15)/interleukin-15 receptor alpha (IL-15Rα) complex, wherein the complex is a fusion protein comprising the amino acid sequence of SEQ ID NO: 9.

16. The IL-2/IL-15Rβγ agonist for use according to any of claims 6 to 11, wherein the IL-2/IL-15Rβγ agonist is the IL-15/IL-15Rα complex, wherein the complex is a fusion protein comprising the amino acid sequence of SEQ ID NO: 9, and the ADC comprises an antibody which specifically binds to HER2, preferably wherein the antibody is trastuzumab.

17. The IL-2/IL-15Rβγ agonist for use according to any of claims 1 to 16, wherein the IL-2/IL-15Rβγ agonist is administered subcutaneously (s.c.) or intraperitoneally (i.p.), preferably s.c.

18. The IL-2/IL-15Rβγ agonist for use according to any of claims 1 to 17, wherein the IL-2/IL-15Rβγ agonist is further combined with an immune checkpoint inhibitor, preferably wherein the checkpoint inhibitor is an anti-PD-1 antibody, an anti-PD-L1 antibody, an anti-PD-L2 antibody, an anti-LAG-3 antibody, an anti-TIM-3 antibody or an anti-CTLA4 antibody, more preferably an anti-PD-L1 antibody or an anti-PD-1 antibody.

19. The IL-2/IL-15Rβγ agonist for use according to any of claims 1 to 18, wherein the cancer is a hematological cancer or a solid cancer, preferably selected from the group consisting of renal cell carcinoma, lung cancer (especially non-small cell lung cancer, small-cell lung cancer), bladder cancer (especially urothelial cancer), melanoma, Merkel-cell carcinoma, skin squamous-cell carcinoma, microsatellite instability high solid tumors, breast cancer (especially triple-negative breast cancer), mesothelioma, prostate cancer, thyroid cancer, thymic cancer, cervical cancer, biliary track cancer, hepatocellular carcinoma, ovarian cancer, gastric cancer, pancreatic cancer, esophageal cancer, head and neck squamous-cell carcinoma, and anal cancer, and ALL, AML, CLL, CML, AMoL, Hodgkin's lymphomas, Non-Hodgkin's lymphomas, and myelomas.
